SEQUENCE LISTING (10 sequences)

SEQ ID NO: 1 - DNA, 597 bp, Homo sapiens
  CDS (1)..(597)
atgaagtcaggcctctggtatttctttctcttctgcttgcgcattaaagttttaacaggagaaatcaatggttctgccaattatgagatgtttatatttcacaacggaggtgtacaaattttatgcaaatatcctgacattgtccagcaatttaaaatgcagttgctgaaaggggggcaaatactctgcgatctcactaagacaaaaggaagtggaaacacagtgtccattaagagtctgaaattctgccattctcagttatccaacaacagtgtctctttttttctatacaacttggaccattctcatgccaactattacttctgcaacctatcaatttttgatcctcctccttttaaagtaactcttacaggaggatatttgcatatttatgaatcacaactttgttgccagctgaagttctggttacccataggatgtgcagcctttgttgtagtctgcattttgggatgcatacttatttgttggcttacaaaaaagtattcatccagtgtgcacgaccctaacggtgaatacatgttcatgagagcagtgaacacagccaaaaaatctagactcacagatgtgaccctataa

SEQ ID NO: 2 - protein, 198 aa, Homo sapiens
MKSGLWYFFLFCLRIKVLTGEINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKYSSSVHDPNGEYMFMRAVNTAKKSRLTDVTL

SEQ ID NO: 3 - DNA, 603 bp, Mus sp.
  CDS (1)..(603)
atgaagccgtacttctgccatgtctttgtcttctgcttcctaatcagacttttaacaggagaaatcaatggctcggccgatcataggatgttttcatttcacaatggaggtgtacagatttcttgtaaataccctgagactgtccagcagttaaaaatgcgattgttcagagagagagaagtcctctgcgaactcaccaagaccaagggaagcggaaatgcggtgtccatcaagaatccaatgctctgtctatatcatctgtcaaacaacagcgtctcttttttcctaaacaacccagacagctcccagggaagctattacttctgcagcctgtccatttttgacccacctccttttcaagaaaggaaccttagtggaggatatttgcatatttatgaatcccagctctgctgccagctgaagctctggctacccgtagggtgtgcagctttcgttgtggtactcctttttggatgcatacttatcatctggttttcaaaaaagaaatacggatccagtgtgcatgaccctaatagtgaatacatgttcatggcggcagtcaacacaaacaaaaagtctagacttgcaggtgtgacctcataa

SEQ ID NO: 4 - protein, 200 aa, Mus sp.
MKPYFCHVFVFCFLIRLLTGEINGSADHRMFSFHNGGVQISCKYPETVQQLKMRLFREREVLCELTKTKGSGNAVSIKNPMLCLYHLSNNSVSFFLNNPDSSQGSYYFCSLSIFDPPPFQERNLSGGYLHIYESQLCCQLKLWLPVGCAAFVVVLLFGCILIIWFSKKKYGSSVHDPNSEYMFMAAVNTNKKSRLAGVTS

SEQ ID NO: 5 - DNA, 909 bp, Homo sapiens
  CDS (1)..(909)
atgcggctgggcagtcctggactgctcttcctgctcttcagcagccttcgagctgatactcaggagaaggaagtcagagcgatggtaggcagcgacgtggagctcagctgcgcttgccctgaaggaagccgttttgatttaaatgatgtttacgtatattggcaaaccagtgagtcgaaaaccgtggtgacctaccacatcccacagaacagctccttggaaaacgtggacagccgctaccggaaccgagccctgatgtcaccggccggcatgctgcggggcgacttctccctgcgcttgttcaacgtcaccccccaggacgagcagaagtttcactgcctggtgttgagccaatccctgggattccaggaggttttgagcgttgaggttacactgcatgtggcagcaaacttcagcgtgcccgtcgtcagcgccccccacagcccctcccaggatgagctcaccttcacgtgtacatccataaacggctaccccaggcccaacgtgtactggatcaataagacggacaacagcctgctggaccaggctctgcagaatgacaccgtcttcttgaacatgcggggcttgtatgacgtggtcagcgtgctgaggatcgcacggacccccagcgtgaacattggctgctgcatagagaacgtgcttctgcagcagaacctgactgtcggcagccagacaggaaatgacatcggagagagagacaagatcacagagaatccagtcagtaccggcgagaaaaacgcggccacgtggagcatcctggctgtcctgtgcctgcttgtggtcgtggcggtggccataggctgggtgtgcagggaccgatgcctccaacacagctatgcaggtgcctgggctgtgagtccggagacagagctcactggccacgtttga

SEQ ID NO: 6 - protein, 302 aa, Homo sapiens
MRLGSPGLLFLLFSSLRADTQEKEVRAMVGSDVELSCACPEGSRFDLNDVYVYWQTSESKTVVTYHIPQNSSLENVDSRYRNRALMSPAGMLRGDFSLRLFNVTPQDEQKFHCLVLSQSLGFQEVLSVEVTLHVAANFSVPVVSAPHSPSQDELTFTCTSINGYPRPNVYWINKTDNSLLDQALQNDTVFLNMRGLYDVVSVLRIARTPSVNIGCCIENVLLQQNLTVGSQTGNDIGERDKITENPVSTGEKNAATWSILAVLCLLVVVAVAIGWVCRDRCLQHSYAGAWAVSPETELTGHV

SEQ ID NO: 7 - DNA, 969 bp, Mus sp.
  CDS (1)..(969)
atgcagctaaagtgtccctgttttgtgtccttgggaaccaggcagcctgtttggaagaagctccatgtttctagcgggttcttttctggtcttggtctgttcttgctgctgttgagcagcctctgtgctgcctctgcagagactgaagtcggtgcaatggtgggcagcaatgtggtgctcagctgcattgacccccacagacgccatttcaacttgagtggtctgtatgtctattggcaaatcgaaaacccagaagtttcggtgacttactacctgccttacaagtctccagggatcaatgtggacagttcctacaagaacaggggccatctgtccctggactccatgaagcagggtaacttctctctgtacctgaagaatgtcacccctcaggatacccaggagttcacatgccgggtatttatgaatacagccacagagttagtcaagatcttggaagaggtggtcaggctgcgtgtggcagcaaacttcagtacacctgtcatcagcacctctgatagctccaacccgggccaggaacgtacctacacctgcatgtccaagaatggctacccagagcccaacctgtattggatcaacacaacggacaatagcctaatagacacggctctgcagaataacactgtctacttgaacaagttgggcctgtatgatgtaatcagcacattaaggctcccttggacatctcgtggggatgttctgtgctgcgtagagaatgtggctctccaccagaacatcactagcattagccaggcagaaagtttcactggaaataacacaaagaacccacaggaaacccacaataatgagttaaaagtccttgtccccgtccttgctgtactggcggcagcggcattcgtttccttcatcatatacagacgcacgcgtccccaccgaagctatacaggacccaagactgtacagcttgaacttacagaccacgcctga

SEQ ID NO: 8 - protein, 322 aa, Mus sp.
MQLKCPCFVSLGTRQPVWKKLHVSSGFFSGLGLFLLLLSSLCAASAETEVGAMVGSNVVLSCIDPHRRHFNLSGLYVYWQIENPEVSVTYYLPYKSPGINVDSSYKNRGHLSLDSMKQGNFSLYLKNVTPQDTQEFTCRVFMNTATELVKILEEVVRLRVAANFSTPVISTSDSSNPGQERTYTCMSKNGYPEPNLYWINTTDNSLIDTALQNNTVYLNKLGLYDVISTLRLPWTSRGDVLCCVENVALHQNITSISQAESFTGNNTKNPQETHNNELKVLVPVLAVLAAAAFVSFIIYRRTRPHRSYTGPKTVQLELTDHA

SEQ ID NO: 9 - DNA, 20 bp, Artificial Sequence
  Description of Artificial Sequence PCR Primer
gactgaagtcggtgcaatgg

SEQ ID NO: 10 - DNA, 22 bp, Artificial Sequence
  Description of Artificial Sequence PCR Primer
ctttctgcctggctaatgctag

(19) United States
(12) Patent Application Publication
(10) Pub. No.: US 2004/0001831 A1
(43) Pub. Date: Jan. 1, 2004

(54) Methods and compositions for the diagnosis and treatment of demyelinating inflammatory disorders

(75) Inventors: James B. Rottman, Sudbury, MA (US); Richard Kroczek, Berlin (DE)

Correspondence Address: PENNIE AND EDMONDS, 1155 AVENUE OF THE AMERICAS, NEW YORK, NY, 100362711

(73) Assignees: Millennium Pharmaceuticals, Inc.; Robert Koch-Institutes

(21) Appl. No.: 10/186,381

(22) Filed: Jun. 26, 2002

(51) Int. Cl. A61K 38/20; A61K 39/395; A61K 31/573; A61K 31/4745; G01N 33/567

(52) U.S. Cl. 424/146.1; 424/85.2; 435/7.21; 514/11; 514/171; 514/291

(57) ABSTRACT

The present invention provides methods of suppressing demyelinating inflammatory disorders in a patient by administering to the patient a compound that inhibits binding of B7RP-1 to ICOS or inhibits signaling through the B7RP-1 pathway. Various therapeutic regimens are provided. Methods of identifying such compounds are also provided. The present invention further provides kits and pharmaceutical compositions useful in the present methods.

### 1. FIELD OF THE INVENTION

The present invention relates to methods of treating or preventing demyelinating inflammatory disorders, in particular demyelinating inflammatory disorders of the central nervous system. Such methods entail administering to a patient in need of such treatment a molecule that inhibits binding of B7RP-1, a B7-related protein, to its receptor, the Inducible Co-Stimulatory molecule (“ICOS”), or a molecule that inhibits signaling through the B7RP-1 pathway. The present invention yet further provides methods of screening for molecules that inhibit binding of B7RP-1 to ICOS and/or that inhibit signaling through the B7RP-1 pathway.

### 2. BACKGROUND OF THE INVENTION

It is widely accepted that optimal T cell activation requires at least two distinct signals delivered during interaction with an antigen-presenting cell (APC); these include antigen-specific signaling through the T cell receptor (TCR) and signaling through costimulatory molecules such as CD28. If the T cell does not receive adequate costimulation, the cell is rendered anergic or undergoes apoptosis. Thus, costimulation is central to T cell activation and survival (Lenschow et al., 1996, Annu. Rev. Immunol. 14:233-258).

CD28 is constitutively expressed on T cells and interacts with the ligands B7-1 and B7-2 on APCs. CD28-mediated costimulation plays a critical role in normal T cell activation, as shown by studies in which the severity of disease in animal models of experimental allergic encephalomyelitis (EAE; Perrin et al., 1999, J. Immunol. 163:1704-1710), collagen-induced arthritis (CIA; Tada et al., 1999, J. Immunol. 162:203-208) and asthma (Mathur et al., 1999, Am. J. Respir. Cell. Mol. Biol. 21:498-509) are markedly reduced when the CD28-B7 pathway is blocked. However, although CD28-mediated costimulation appears to be essential for initial T cell priming, secondary or memory responses are CD28-independent, which suggests the presence of alternative costimulatory pathways (Kopf et al., 2000, J. Exp. Med. 192:53-61).

One such alternative T cell costimulatory pathway involves the inducible costimulatory molecule (ICOS). Although not constitutively expressed, ICOS is rapidly up-regulated on T lymphocytes upon activation through the CD28 pathway (McAdam et al., 2000, J. Immunol. 165:5035-5040) or via activation with phorbol 12-myristate 13-acetate (PMA)-ionomycin (Yoshinaga et al., 1999, Nature 402:827-832) or anti-CD3 (Mages et al., 2000, Eur. J. Immunol. 30:1040-1047). ICOS is expressed on both CD4⁺ and CD8⁺ T cells, but polarized T helper 2 (T_{H}2) cells express more ICOS mRNA than polarized T_{H}1 cells (Coyle et al., 2000, Immunity 13:95-105). ICOS shares 19% homology with CD28 and binds to the ligand B7RP-1, which is expressed on B cells and macrophage (Yoshinaga et al., 2000, Nature 402:827-832). In CD28-deficient mice, ICOS provides T cell costimulation for immune responses to viruses and intestinal parasites (Kopf et al., 2000, J. Exp. Med. 192, 53-61). ICOS costimulation also induces interleukin 10 (IL-10) expression, CD40 ligand (CD40L) up-regulation and TH function for B cell maturation (Hutloff et al., 1999, Nature 397:263-266; McAdam et al., 2001, Nature 409:102-105. In addition, T cells from ICOS-deficient mice proliferate less when cultured with anti-CD3 and show deficiencies in IL-4 production, and ICOS-deficient mice have abnormal germinal center formation in the spleen (Dong et al., 2001, Nature 409:97-101; Tafari et al., 2001, Nature 409:105-109). Thus, ICOS appears to play an important role in both T and B cell function.

Experimental allergic encephalomyelitis (EAE), the primary recognized animal model of multiple sclerosis, is initiated by immunizing susceptible strains of mice with specific myelin proteins such as myelin oligodendrocyte glycoprotein (MOG) or immunodominant myelin peptides such as MOG 35-55 or proteolipid protein peptide (PLP) 139-151 (Maron, 1999, Int Immunol 11:1573-80; Slavin, 1998, Autoimmunity 28:109-20; Wekerle, 1991, Acta Neurol (Napoli) 13:197-204). The immune response to these myelin antigens can be divided into afferent and efferent phases. During the afferent phase, myelin antigens are “processed” by antigen presenting cells (APC's) in regional lymph nodes and presented in the context of major histocompatibility class II (MHC II) molecules to naïve myelin-specific CD4+ T cells (Slavin, 2001, J. Clin. Invest. 108:1133-9). The interaction of the MHC II molecule with the T cell receptor (TCR) sends an activation signal to the cell, ultimately resulting in differentiation into an encephalitogenic effector T cell. During the efferent phase of the disease, the encephalitogenic T cells traffic to the brain and are further activated in situ through the TCR to mediate disease. However, during both afferent and efferent phases of the disease, T cells must receive a second signal through a costimulatory molecule in order to become fully activated. Molecules that inhibit the costimulatory signal and therefore likely to be useful therapeutic candidates for the treatment of inflammatory demyelinating diseases such as multiple sclerosis.

### 3. SUMMARY OF THE INVENTION

The present invention provides methods and compositions useful to treat or prevent demyelinating inflammatory disorders, particularly demyelinating inflammatory disorders of the central nervous system, such as multiple sclerosis. The present invention is based on the discovery that endothelial cells in the blood-brain barrier (hereinafter, “BBB”) express B7RP-1, and that T lymphocytes that mediate inflammation in the central nervous system (“CNS”) require co-stimulation by endothelial cells of the BBB through the ICOS-B7RP-1 pathway to traverse the BBB and mediate inflammation. Therefore, agents that inhibit the interaction between ICOS and B7RP-1 or otherwise inhibit signaling through the ICOS-B7RP-1 pathway (referred to herein as “ICOS-B7RP-1 inhibitors”) are useful reagents to block entry of activated lymphocytes into the brain and thereby are useful reagents to inhibit (e.g., prevent or treat) inflammation.

Accordingly, the invention provides methods of treating or preventing a demyelinating inflammatory disorder of the central nervous system in a patient, comprising administering to the patient in need of such treatment an ICOS-B7RP-1 inhibitor in an amount effective for treating the demyelinating inflammatory disorder. In one embodiment, the patient is human. In another embodiment, the ICOS-B7RP-1 inhibitor is an ICOS polypeptide or a B7RP-1-binding portion thereof. In another embodiment, the ICOS-B7RP-1 inhibitor is a soluble ICOS polypeptide. In another embodiment, the ICOS-B7RP-1 inhibitor is a B7RP-1 polypeptide or an ICOS-binding portion thereof. In another embodiment, the ICOS-B7RP-1 inhibitor is a soluble B7RP-1 polypeptide. In another embodiment, the ICOS-B7RP-1 inhibitor is an anti-ICOS antibody. In another embodiment, the ICOS-B7RP-1 inhibitor is an anti-B7RP-1 antibody. In another embodiment, the ICOS-B7RP-1 inhibitor does not induce ICOS-B7RP-1 signaling. In another embodiment, the demyclinating inflammatory disorder is multiple sclerosis.

In another embodiment, the ICOS-B7RP-1 inhibitor is administered during a period of relapse in said patient. In another embodiment, the ICOS-B7RP-1 inhibitor is administered during a period of remission in said patient. In another embodiment, the ICOS-B7RP-1 inhibitor is administered during chronic progressive multiple sclerosis in said patient.

In certain embodiments, the method may further comprise administering a second therapeutic agent selected from the group consisting of an immunosuppressive agent and a biological response modifier. In another embodiment, the second therapeutic agent is an immunosuppressive agent. In another embodiment, the immunosuppressive agent is cyclosporine, FK506, rapamycin, or prednisone. In another embodiment, the second therapeutic agent is a biological response modifier. In another embodiment, the biological response modifier is an interleukin. In another embodiment, the interleukin is interleukin 4. In another embodiment, the biological response modifier is an antibody. In another embodiment, the antibody is immunospecific to CCR1, RANTES, MCP-1, MIP-2, Interleukin-1α, Interleukin-1β, Interleukin-6, Interleukin-12p35 or IFN-γ.

In certain embodiments, the second therapeutic agent is administered concurrently with the ICOS-B7RP-1 inhibitor. In another embodiment, the ICOS-B7RP-1 inhibitor and the second therapeutic agent are administered during a period of relapse in said patient. In yet another embodiment, the ICOS-B7RP-1 inhibitor and the second therapeutic agent are administered during a period of remission in said patient. In another embodiment, the ICOS-B7RP-1 inhibitor and the second therapeutic agent are administered during chronic progressive multiple sclerosis in said patient. In another embodiment, the second therapeutic agent and the ICOS-B7RP-1 inhibitor are administered successively. In another embodiment, the second therapeutic agent is administered prior to administration of the ICOS-B7RP-1 inhibitor. In another embodiment, the second therapeutic agent is administered after administration of the ICOS-B7RP-1 inhibitor. In another embodiment, the ICOS-B7RP-1 inhibitor and the second therapeutic agent are both administered during a period of relapse in said patient. In another embodiment, the ICOS-B7RP-1 inhibitor and the second therapeutic agent are both administered during a period of remission in said patient. In another embodiment, the ICOS-B7RP-1 inhibitor and the second therapeutic agent are both administered during chronic progressive multiple sclerosis in said patient. In another embodiment, the ICOS-B7RP-1 inhibitor is administered during a period of relapse in said patient and the second therapeutic agent is administered during a period of remission in said patient. In another embodiment, the ICOS-B7RP-1 inhibitor is administered during a period of remission in said patient and the second therapeutic agent is administered during a period of relapse in said patient.

The invention also provides a method of identifying a candidate ICOS-B7RP-1 inhibitor, comprising (a) contacting an ICOS polypeptide with a B7RP-1 polypeptide expressed on an endothelial cell surface and the test compound, under conditions that, in the absence of the test compound, allow the ICOS polypeptide to bind to the B7RP-1 polypeptide and thereby form an ICOS-B7RP-1 complex; and (b) determining whether ICOS-B7RP-1 complex formation is inhibited by the test compound; wherein inhibition of ICOS-B7RP-1 complex formation by the test compound identifies the test compound as a candidate ICOS-B7RP-1 inhibitor. In certain embodiments, the ICOS polypeptide is contacted with the B7RP-1 polypeptide prior to contacting the ICOS polypeptide with the test compound. In another embodiment, the ICOS polypeptide is contacted with the test compound prior to contacting the ICOS polypeptide with the B7RP-1 polypeptide. In another embodiment, the B7RP-1 polypeptide is contacted with the test compound prior to contacting the ICOS polypeptide B7RP-1 polypeptide and the test compound. In another embodiment, the ICOS polypeptide is expressed on a cell. In another embodiment, the cell is a T cell. In another embodiment, the ICOS polypeptide is immobilized on a solid surface. In another embodiment, the ICOS polypeptide is present in a cell membrane, which cell membrane is immobilized on the solid surface. In another embodiment, the ICOS polypeptide is directly immobilized on the solid surface. In another embodiment, determining whether ICOS-B7RP-1 complex formation is inhibited by the test compound comprises measuring the amount binding between ICOS and In another embodiment, the amount of binding is measured by ELISA. In another embodiment, determining whether ICOS-B7RP-1 complex formation is inhibited by the test compound comprises measuring ICOS-B7RP-1 pathway activation. In another embodiment, measuring ICOS-B7RP-1 pathway activation comprises measuring ICOS activity.

The invention also provides a method of identifying a candidate ICOS-B7RP-1 inhibitor, comprising (a) identifying a test compound as a candidate ICOS-B7RP-1 inhibitor by the above-described methods of the invention; (b) contacting a T-cell capable of being activated by B7RP-1 with B7RP-1, wherein the B7RP-1 expressed on an endothelial cell surface, in the presence of the test compound; and (c) determining whether a lower level of ICOS-B7RP-1 activity occurs in the T-cell after said contacting relative to a control T-cell contacted with B7RP-1 in the absence of the test compound; wherein a lower level of activity identifies the test compound as a candidate ICOS-B7RP-1 inhibitor. In one embodiment, determining whether a lower level of ICOS-B7RP-1 activity occurs in the T-cell comprises measuring ICOS pathway activation. In another embodiment, determining whether a lower level of ICOS-B7RP-1 activity occurs in the T-cell comprises measuring T cell activation. In another embodiment, the method is performed in vitro. In another embodiment, the method is performed in vivo. In another embodiment, T-cell activation is indicated by an increase in the expression of MCP-1, CCR1, interleukin-1α, interleukin-1β, interleukin-6, interleukin-10, or interferon-γ. In another embodiment, T cell activation is evidenced by the ability of the T cell to traverse an in vitro model of the blood brain barrier.

The invention further provides a method of identifying a candidate ICOS-B7RP-1 inhibitor, comprising (a) identifying a test compound as a candidate ICOS-B7RP-1 inhibitor by any of the foregoing methods; (b) administering to a model animal with experimental allergic encephalomyelitis the test compound during the efferent stage of said experimental allergic encephalomyelitis; and (c) determining whether the test compound abrogates a central nervous system phenotype of experimental allergic encephalomyclitis, wherein abrogation of a central nervous system phenotype of experimental allergic encephalomyelitis identifies the test compound as a candidate ICOS-B7RP-1 inhibitor. In one embodiment, determining whether the test compound abrogates a central nervous system phenotype of experimental allergic encephalomyelitis comprises determining whether ICOS positive T cells traverse the blood brain barrier of said model animal at a reduced rate relative to a model animal with experimental allergic encephalomyelitis to whom the test compound is not administered. In another embodiment, determining whether the test compound abrogates a central nervous system phenotype of experimental allergic encephalomyelitis comprises determining whether brain inflammation is reduced in said model animal relative to a model animal with experimental allergic encephalomyelitis to whom the test compound is not administered In another embodiment, determining whether the test compound abrogates a central nervous system phenotype of experimental allergic encephalomyelitis comprises determining whether physical symptoms of experimental allergic encephalomyelitis are reduced in the model animal relative to a model animal with experimental allergic encephalomyelitis to whom the test compound is not administered.

The invention also provides a method of identifying a candidate ICOS-B7RP-1 inhibitor, comprising (a) contacting a T-cell capable of being activated by B7RP-1 with B7RP-1, wherein the B7RP-1 expressed on an endothelial cell surface, in the presence of a test compound; and (b) determining whether a lower level of ICOS-B7RP-1 activity occurs in the T-cell after said contacting relative to a control T-cell contacted with B7RP-1 in the absence of the test compound; wherein a lower level of activity identifies the test compound as a candidate ICOS-B7RP-1 inhibitor. In one embodiment, determining whether a lower level of ICOS-B7RP-1 activity occurs in the T-cell comprises measuring ICOS pathway activation. In another embodiment, determining whether a lower level of ICOS-B7RP-1 activity occurs in the T-cell comprises measuring T cell activation. In another embodiment, the method is performed in vitro. In another embodiment, the method is performed in vivo. In another embodiment, the ICOS pathway activation is indicated by an increase in the expression of the ICOS gene. In another embodiment, the expression of the ICOS gene is measured by a method comprising measuring the expression of ICOS mRNA or ICOS protein. In another embodiment, the expression of the ICOS gene is measured by a method comprising measuring the expression of a reporter gene under the control of an ICOS regulatory sequence. In another embodiment, the T-cell activation is indicated by an increase in the expression of MCP-1, CCR1, interleukin-1α, interleukin-1β, interleukin-6, interleukin-10, or interferon-γ. In another embodiment, T cell activation is evidenced by the ability of the T cell to traverse an in vitro model of the blood brain barrier.

The invention also provides a method of identifying a candidate ICOS-B7RP-1 inhibitor, comprising (a) identifying a test compound as a candidate ICOS-B7RP-1 inhibitor by any of the foregoing methods; (b) administering to a model animal with experimental allergic encephalomyelitis the test compound during the efferent stage of said experimental allergic encephalomyelitis; and (c) determining whether the test compound abrogates a central nervous system phenotype of experimental allergic encephalomyelitis, wherein abrogation of a central nervous system phenotype of experimental allergic encephalomyclitis identifies the test compound as a candidate ICOS-B7RP-1 inhibitor. In one embodiment, determining whether the test compound abrogates a central nervous system phenotype of experimental allergic encephalomyelitis comprises determining whether ICOS positive T cells traverse the blood brain barrier of said model animal at a reduced rate relative to a model animal with experimental allergic encephalomyelitis to whom the test compound is not administered. In another embodiment, determining whether the test compound abrogates a central nervous system phenotype of experimental allergic encephalomyelitis comprises determining whether brain inflammation is reduced in said model animal relative to a model animal with experimental allergic encephalomyelitis to whom the test compound is not administered. In another embodiment, determining whether the test compound abrogates a central nervous system phenotype of experimental allergic encephalomyelitis comprises determining whether physical symptoms of experimental allergic encephalomyelitis are reduced in the model animal relative to a model animal with experimental allergic encephalomyelitis to whom the test compound is not administered.

In addition, the invention provides a method of identifying a candidate ICOS-B7RP-1 inhibitor, comprising (a) administering to a model animal with experimental allergic encephalomyelitis a test compound during the efferent stage of said experimental allergic encephalomyelitis; and (b) determining whether the test compound abrogates a central nervous system phenotype of experimental allergic encephalomyelitis, wherein abrogation of a central nervous system phenotype of experimental allergic encephalomyelitis identifies the test compound as a candidate ICOS-B7RP-1 inhibitor. In one embodiment, determining whether the test compound abrogates a central nervous system phenotype of experimental allergic encephalomyelitis comprises determining whether ICOS positive T cells traverse the blood brain barrier of said model animal at a reduced rate relative to a model animal with experimental allergic encephalomyelitis to whom the test compound is not administered. In another embodiment, determining whether the test compound abrogates a central nervous system phenotype of experimental allergic encephalomyelitis comprises determining whether brain inflammation is reduced in said model animal relative to a model animal with experimental allergic encephalomyelitis to whom the test compound is not administered. In another embodiment, determining whether the test compound abrogates a central nervous system phenotype of experimental allergic encephalomyelitis comprises determining whether physical symptoms of experimental allergic encephalomyelitis are reduced in the model animal relative to a model animal with experimental allergic encephalomyelitis to whom the test compound is not administered. In another embodiment, the model animal is a mouse.

In certain embodiments of the invention disclosed hereinabove, the method may further comprise, prior to step (a), identifying a suitable test compound by a method comprising (a) contacting an ICOS polypeptide with a B7RP-1 polypeptide and a molecule, under conditions that, in the absence of the molecule, allow the ICOS polypeptide to bind to the B7RP-1 polypeptide and thereby form an ICOS-B7RP-1 complex; and (b) determining whether ICOS-B7RP-1 complex formation is inhibited by the molecule; wherein inhibition of ICOS-B7RP-1 complex formation by the molecule identifies the molecule as a suitable test compound. In another embodiment, the ICOS polypeptide is contacted with the B7RP-1 polypeptide prior to contacting the ICOS polypeptide with the molecule. In another embodiment, the ICOS polypeptide is contacted with the molecule prior to contacting the ICOS polypeptide with the B7RP-1 polypeptide. In another embodiment, the B7RP-1 polypeptide is contacted with the molecule prior to contacting the ICOS polypeptide with the B7RP-1 polypeptide and the test compound.

I In the foregoing methods of identifying candidate ICOS-B7RP-1 inhibitors, unless otherwise indicated, the terms “ICOS polypeptide” and “B7RP-1 polypeptide” refer to polypeptides comprising a B7RP-1-binding domain of ICOS and an ICOS-binding domain of B7RP-1, respectively.

### 4. DETAILED DESCRIPTION OF THE INVENTION

One of the more recently described costimulatory molecules, the inducible costimulator (ICOS), is upregulated on activated T cells and has been shown to play important roles in the immunopathogenesis of asthma and allograft rejection (Hutloff et al., 1999, Nature 397:263-266; McAdam et al., 2001, Nature 409:102-105; Dong et al., 2001, Nature 409:97-101; Yoshinaga, 1999, Nature 402:827-832; Özkaynak et al., 2001, Nature Immunol. 2:591-596; Tafari et al., 2001, Nature 409:105-109; Gonzalo, 2001, Nat. Immunol. 2(7):597-604).

The present inventors have identified a novel role for the ICOS pathway in the immunopathogenesis of inflammatory demyelinating diseases such as allergic encephalomyelitis/multiple sclerosis. The immune response in EAE, the primary model of multiple sclerosis, can be divided into afferent and efferent phases. During the afferent phase, myelin antigens are “processed” by antigen presenting cells (APC's) in regional lymph nodes and presented in the context of major histocompatibility class II (MHC II) molecules to naïve myelin-specific CD4+ T cells (Slavin, 2001, J. Clin. Invest. 108:1133-9). The interaction of the MHC II molecule with the T cell receptor (TCR) sends an activation signal to the cell, ultimately resulting in differentiation into an encephalitogenic effector T cell. During the efferent phase of the disease, the encephalitogenic T cells traffic to the brain and are further activated in situ through the TCR to mediate disease.

The first set of studies described in Section 5.2 herein demonstrate that ICOS also plays an important role during both the afferent and efferent phases of EAE. For example, ICOS blockade during the afferent phase of EAE causes enhanced disease symptoms, resulting at least in part from TH1 polarization. In contrast, ICOS blockade during the efferent phase of the immune response abrogates disease onset. This study further demonstrates that ICOS+ T lymphocytes arrive in the brain by day 10 PI (i.e., following injection of PLP), prior to onset of EAE symptoms, and that these ICOS+ lymphocytes comprise less that 12% of all brain-infiltrating T cells. Based upon these observations and the fact that ICOS blockade during the efferent phase of the immune response (days 9-20 PI) abrogated disease, a second study, described in Section 5.3 below, was conducted to determine whether the encephalitogenic CD4+ T cells are contained within the ICOS+subset and whether the ICOS/B7RP-1 pathway is critical for costimulation of these cells. This second study provides evidence that encephalitogenic CD4+ T cells are contained within the ICOS+ population, that these cells may be activated at the level of the blood-brain barrier (BBB) prior to entry into the brain and that blockade of the ICOS/B7RP-1 costimulatory pathway during efferent disease inhibits opening of the BBB. These data suggest that inhibition of the ICOS/B7RP-1 pathway is a suitable approach for the treatment of demyelinating inflammatory disorders, particularly those that involve infiltration of T lymphocytes into the brain across the blood brain barrier, for example multiple sclerosis.

Accordingly, the present invention provides methods useful to treat or prevent demyelinating inflammatory disorders, particularly demyelinating inflammatory disorders of the nervous system. Such methods are described in more detail in Section 4. 1, infra.

The present invention provides pharmaceutical compositions and kits that are useful for practicing the methods of the invention. Such pharmaceutical compositions and kits are described in Sections 4.6 and 4.7 below, respectively.

The present invention further encompasses methods of identifying a class of molecules referred to herein as “ICOS-B7RP-1 inhibitors,” which molecules block the interaction of ICOS and B7RP-1 and/or inhibit signaling through the ICOS-B7RP-1 pathway. Such methods are described in Section 4.5, infra. The present invention yet further encompasses kits that are useful in practicing the screening methods of the present application. Such kits are described in Section 4.6, infra.

### 4.1 METHODS OF THE INVENTION

Described below are methods for treating or preventing demyelinating inflammatory disorders, particularly demyelinating inflammatory disorders of the central nervous system, and related compositions. The methods of the invention involve the administration of an agent which inhibits the ICOS-B7RP-1 pathway, i.e., an agent that either partially or fully prevents or inhibits or disrupts the interaction between the ICOS receptor and its ligand, B7RP-1, or partially or fully inhibits signaling through the ICOS-B7RP-1 pathway, to a patient in need of such prevention or treatment. Such an agent is referred to herein as an ICOS-B7RP-1 inhibitor.

The ICOS-B7RP-1 inhibitor can be a competitive or non-competitive inhibitor of the ICOS-B7RP-1 interaction. As used herein, a non-competitive inhibitor is a molecule that binds to an ICOS-B7RP-1 complex and blocks, at least partially, signaling through the pathway. A competitive inhibitor is one that binds to either ICOS or B7RP-1 and inhibits, at least partially, ICOS-B7RP-1 complex formation.

As described in Section 4.2, infra, the ICOS-B7RP-1 inhibitor can be a protein. In one embodiment, the ICOS-B7RP-1 inhibitor is a membrane-bound form of B7RP-1 or ICOS, for example B7RP-1 or ICOS recombinantly expressed on a cell. For example, an ICOS-expressing cell that does not contain the machinery for mediating the B7RP-1 signal can be used to inhibit the endogenous ICOS-B7RP-1 interaction. In more preferred embodiments, the ICOS-B7RP-1 inhibitor is a soluble protein. In one embodiment, the ICOS-B7RP-1 inhibitor is a soluble form of ICOS or a soluble form of another receptor to which B7RP-1 binds. In another embodiment, the ICOS-B7RP-1 inhibitor is a soluble B7RP-1 protein or another ligand which binds to ICOS. In yet other embodiments, the ICOS-B7RP-1 inhibitor is an anti-ICOS or anti-B7RP-1 antibody. Alternatively, the ICOS-B7RP-1 inhibitor can be small organic or inorganic molecule of preferably less than 500 daltons in size.

The outcome of the present therapeutic and prophylactic methods is to at least produce in a patient a healthful benefit, which includes but is not limited to: prolonging the lifespan of a patient, prolonging the onset of symptoms of the disorder (for example by prolonging the onset of initial symptoms of the disorder and/or by prolonging the onset of relapses of the disorder) and/or prolonging the onset of a more advanced stage of the disorder and/or alleviating a symptom of the disorder after onset of a symptom of the disorder.

As used herein, the terms “treat”, “treatment”, and “therapy” refer to administration of the ICOS-B7RP-1 inhibitor to the patient after the onset of symptoms or molecular indications of the demyelinating inflammatory disorder. In contrast, the terms “prevent”, “prevention” and “prophylaxis” refer to administration of the ICOS-B7RP-1 inhibitor to the patient before the onset of symptoms or molecular indications of the demyelinating inflammatory disorder of interest

The invention provides methods of treating or preventing a demyelinating inflammatory disorder of the central nervous system in a patient, for example a human patient, said methods comprising administering to the patient in need of such treatment an ICOS-B7RP-1 inhibitor in an amount effective for treating the demyelinating inflammatory disorder. Suitable ICOS-B7RP-1 inhibitors are described in Section 4.2 below, or can be identified according to the methods described in Section 4.5.

Preferably, an ICOS-B7RP-1 inhibitor is administered in the present methods in purified form. As, used herein, purified form means that the ICOS-B7RP-1 inhibitor is at least 30%, more preferably at least 40%, and yet more preferably at least 50% pure. In specific embodiments, the ICOS-B7RP-1 inhibitor is 60%, 70%, 80%, 90%, 95% or 98% pure.

Multiple sclerosis, a preferred exemplary disorder of the invention, is a chronic inflammatory disease of the central nervous system and is associated with periods of disability (relapse) alternating with periods of recovery (remission), and often results in chronic progressive multiple sclerosis characterized by neurologic disability (Brod et al., 1996, Am Fam Physician 54(4):1301-6 and 1309-11). The therapeutic methods of the present invention can be practiced during any of these periods, and are preferably practiced during peak periods of relapse. For example, in certain embodiments, an ICOS-B7RP-1 inhibitor is administered during a period of relapse in a patient with multiple sclerosis. In other embodiments, the ICOS-B7RP-1 inhibitor is administered during a period of remission in such a patient. In yet other embodiments, the ICOS-B7RP-1 inhibitor is administered during chronic progressive multiple sclerosis in the patient.

ICOS-B7RP-1 inhibitors that are antibodies can be engineered for optimal stability upon administration to the patient. Preferred antibodies, particularly those for use in single therapy, have generally a half life of 4-144 hours, more preferably about 6-120 hours, and most preferably about 8-96 hours in a patient. In certain specific embodiments, such antibodies have a half life of 4-12, 4-24, 8-24, 8-36, 8-48, 12-24, 12-36, or 12-48. Thus, in certain embodiments, administration of an antibody with that is sufficiently stable for treating a relapse of multiple sclerosis but not excessively stable that it is present during the afferent stage of a successive relapse of the disease, is a desired goal of the present invention.

The ICOS-B7RP-1 inhibitors of the present invention can be administered alone or in combination with a second therapeutic agent, for example as described in Section 4.3 below.

Techniques such as magnetic resonance imaging, spectroscopy and electrophysiological techniques can be used to stage the disease in a patient. Such techniques may be employed to assess whether a therapeutic regimen of the invention (entailing the administration of an ICOS-B7RP-1 inhibitor alone or in combination therapy as described in Section 4.3 below) should be initiated. The earliest detectable event in the development of a new lesion is an increase in permeability of the blood-brain barrier associated with inflammation (McDonald, 1994, J. Neuropathol. Exp. Neurol. 53(4):338-43). Generally, once such a system is detected, a patient can undergo treatment with an ICOS-B7RP-1 inhibitor.

The patients on whom the methods of the invention are practiced include, but are not limited to, animals such as cows, pigs, horses, chickens, cats, dogs, etc., and are preferably mammals, and most preferably human.

The therapeutic regimens of the present invention can be practiced as long as the treatment or prevention of a demyelinating inflammatory disorder is required or desired.

### 4.1.1 DISORDERS OF THE INVENTION

The methods and compositions of the present invention are useful for treating or preventing a variety of demyelinating inflammatory disorders of the central nervous system. In one embodiment, such demyelinating inflammatory disorders, such as multiple sclerosis, have an autoimmune pathology. Such disorders are referred to herein as disorders of the invention.

Demyelinating inflammatory disorders of the invention include, but are not limited to, allergic encephalomyelitis, systemic lupus erythematosus (“SLE”), and multiple sclerosis.

### 4.2 ICOS-B7RP-1 INHIBITORS

As discussed above, an ICOS-B7RP-1 inhibitor is a molecule that prevents the interactions between ICOS and B7RP-1 and/or inhibits signaling through the ICOS-B7RP-1 pathway. Many types of molecules can be used as ICOS-B7RP-1 inhibitors. Such molecules include polypeptides, peptides, antibodies, and small molecules.

In certain embodiments, an ICOS-B7RP-1 inhibitor preferably inhibits the complex formation between the ICOS receptor and its ligand B7RP-1 by at least 20%, more preferably by at least 30%, more preferably by at least 40%, yet more preferably by at least 50%. In certain embodiments, an ICOS-B7RP-1 inhibitor inhibits the ICOS-B7RP-1 pathway by up to 60%, 70%, 80%, or 90%. As used herein, percentage inhibition of ICOS-B7RP-1 complex formation is measured according to an embodiment of the heterogenous assay described in Section 4.5, infra. Briefly, a protein (such as a fusion protein) comprising a B7RP-1-binding portion of ICOS (or an ICOS-binding portion of B7RP-1) is immobilized on a solid surface, and contacted with a protein comprising an ICOS-binding portion of B7RP-1 (or a B7RP-1-binding portion of ICOS) in the presence and absence of the test compound. After the reaction is complete, unreacted components are removed (e.g, by washing) and any complexes formed will remain immobilized on the solid surface. A radioactively labeled antibody that binds to the ICOS-binding portion of B7RP-1 (or to the B7RP-1-binding portion of ICOS), but not to the test compound, can be added to the system and allowed to bind to the complexed components. The interaction between ICOS and B7RP-1 can be detected by measuring the amount of radioactivity that remains associated with the ICOS-B7RP-1 complex. A successful inhibition of the interaction by the test compound will result in a decrease in measured radioactivity. The percent inhibition of the ICOS -B7RP-1 interaction is the percentage difference in bound radioactivity in the present and absence of test compound; for example, if the amount of bound radioactivity in the presence of the test compound is 70% of bound radioactivity in the absence of the test compound, the test compound is said to inhibit the ICOS-B7RP-1 interaction by 30%.

The ICOS-B7RP-1 inhibitor can be a competitive or non-competitive inhibitor of the ICOS-B7RP-1 interaction. The ICOS-B7RP-1 inhibitor can be a competitive or non-competitive inhibitor of the ICOS-B7RP-1 interaction. As used herein, a non-competitive inhibitor is a molecule that binds to an ICOS-B7RP-1 complex and blocks, at least partially, signaling through the pathway. A competitive inhibitor is one that binds to either ICOS or B7RP-1 and inhibits, at least partially, ICOS-B7RP-1 complex formation.

In certain embodiments, the ICOS-B7RP-1 inhibitor is a protein. In one embodiment, the ICOS-B7RP-1 inhibitor is a membrane-bound form of B7RP-1 or ICOS, for example B7RP-1 or ICOS naturally or recombinantly expressed on a cell. For example, an ICOS-expressing cell that does not mediate an inflammatory response can be used to inhibit the endogenous ICOS-B7RP-1 interaction. In more preferred embodiments, the ICOS-B7RP-1 inhibitor is a soluble protein. In one embodiment, the ICOS-B7RP-1 inhibitor is a soluble form of ICOS. In another embodiment, the ICOS-B7RP-1 inhibitor is a soluble B7RP-1 protein. In yet other embodiments, the ICOS-B7RP-1 inhibitor is an anti-ICOS or anti-B7RP-1 antibody. Alternatively, the ICOS-B7RP-1 inhibitor can be small organic or inorganic molecule of preferably less than 500 daltons in size.

The present invention also encompasses methods for designing new agents that are ICOS-B7RP-1 inhibitors, wherein these new agents may include, but not be limited to, any agent with the ability to inhibit the interaction between ICOS and B7RP-1 or otherwise inhibit signaling through the ICOS-B7RP-1 pathway, or to inhibit signaling through the B7RP-1 pathway. Such an agent would include, but not be limited to, monoclonal antibodies and antisense compounds of the invention capable of being delivered intracellularly. The choice of agent and calculation of optimal dosage, although highly individualized, may be carried out according to methods commonly known in the art.

The present invention further provides a method of performing rational drug design to develop drugs that can inhibit the interaction between ICOS and B7RP-1 or otherwise inhibit signaling through the ICOS-B7RP-1 pathway, or inhibit signaling through the B7RP-1 pathway, and can thereby ameliorate a disorder of the invention. Such rational drug design can be performed using compounds that have been identified as ICOS-B7RP-1 inhibitors as a starting point. Thus, the present invention provides screens and assays to allow more specific inhibitors to be identified. Such methods of rational drug design are well-known in the art.

For example, potential modulators can be examined through the use of computer modeling using a docking program such as GRAM, DOCK, or AUTODOCK (Dunbrack et al., Folding & Design 2:27-42 (1997)), to identify potential modulators of, e.g., an ICOS-B7RP-1 pathway. These modulators can then be tested for their effect on ICOS and/or B7RP-1 activity. This procedure can include computer fitting of potential modulators to the ICOS-B7RP-1 complex to ascertain how well the shape and the chemical structure of the potential modulator will bind to either ICOS and/or B7RP-1 (Bugg et al., 1993, Scientific American (December) 269(6):92-98; West et al., TIPS, 16:67-74 (1995)). Computer programs can also be employed to estimate the attraction, repulsion, and steric hindrance of the subunits with a modulator/inhibitor.

Generally the tighter the fit, the lower the steric hindrances, and the greater the attractive forces, the more potent the potential modulator since these properties are consistent with a tighter binding constant. Furthermore, the more specificity in the design of a potential drug the more likely that the drug will not interact as well with other proteins. This will minimize potential side-effects due to unwanted interactions with other proteins.

Initially, compounds known to bind to ICOS or B7RP-1 or known to be ICOS-B7RP-1 inhibitors can be systematically modified by computer modeling programs until one or more promising potential analogs are identified. In addition, systematic modification of selected analogs can then be systematically modified by computer modeling programs until one or more potential analogs are identified. Such analyses are well known to those of skill in the art and have been shown to be effective in the development of, e.g., HIV protease inhibitors (see, e.g., Lam et al., Science 263:380-384 (1994); Wlodawer et al., Ann. Rev. Biochem. 62:543-585 (1993); Appelt, Perspectives in Drug Discovery and Design 1:23-48 (1993); Erickson, Perspectives in Drug Discovery and Design 1:109-128 (1993)). Alternatively a potential ICOS-B7RP-1 inhibitor can be obtained by initially screening a random peptide library produced by recombinant bacteriophage, e.g., as disclosed hereinabove. A peptide selected in this manner is then systematically modified by computer modeling programs as disclosed above, and then treated analogously to a structural analog as disclosed above.

Once a potential ICOS-B7RP-1 inhibitor is identified, it can be either selected from a library of chemicals, as are commercially available (e.g., from Chembridge Corporation, San Diego, Calif. or Evotec OAI, Abingdon, UK). Alternatively, the potential ICOS-B7RP-1 inhibitor may be synthesized de novo. Potential peptide modulators may be synthesized by protein synthetic techniques, e.g., by use of a peptide synthesizer or other methods of protein/peptide synthesis well known in the art. The de novo synthesis of one or even a relatively small group of specific compounds is reasonable in the art of drug design.

Furthermore, any of the potential agents (or targets for the potential agents, e.g., ICOS or B7RP-1) can be labeled. Suitable labels include enzymes (e.g., alkaline phosphatase or horseradish peroxidase), fluorophores (e.g., fluorescein isothiocyanate (FITC), phycoerythrin (PE), Texas red (TR), rhodamine, free or chelated lanthanide series salts, especially Eu³⁺, to name a few fluorophores), chromophores, radioisotopes, chelating agents, dyes, colloidal gold, latex particles, ligands (e.g., biotin), chemiluminescent agents, magnetic beads or magnetic resonance imaging labels. When a control marker is employed, the same or different labels may be used for the receptor and control marker.

In embodiments wherein a radioactive label, such as the isotopes ³H, ¹⁴C, ³²P, 35S, ³⁶Cl, ⁵¹Cr, ⁵⁷Co, ⁵⁸Co, ⁵⁹Fe, ⁹⁰Y, ¹²⁵I ¹³¹I, and ¹⁸⁶Re is used, standard counting procedures known in the art may be utilized.

In embodiments wherein the label is an enzyme, detection may be accomplished by any of the presently utilized colorimetric, spectrophotometric, fluorospectrophotometric, amperometric or gasometric techniques known in the art.

A direct label is an example of a label that can be used according to the methods of the present invention. A direct label is an entity that, in its natural state, is readily visible, either to the naked eye (for example, by visual inspection through a compound or dissecting light microscope), or with the aid of an optical filter and/or applied stimulation, e.g., U.V. light to promote fluorescence. Examples of colored labels that can be used according to the methods of the present invention, include metallic sol particles, for example, gold sol particles such as those disclosed by Leuvering (U.S. Pat. No. 4,313,734); dye sol particles such as disclosed by Gribnau et al. (U.S. Pat. No. 4,373,932) and May et al. (WO 88/08534); dyed latex such as disclosed by May et al. (WO 88/08534), Snyder (EP-A 0280 559 and 0 281 327); or dyes encapsulated in liposomes as disclosed by Campbell et al. (U.S. Pat. No. 4,703,017).

Other direct labels include a radionucleotide, a luminescent moiety, or a fluorescent moiety including, but not limited, to, e.g., green fluorescent protein (GFP) or a modified/fusion chimera of green fluorescent protein (GFP) (as disclosed in U.S. Pat. No. 5,625,048, issued Apr. 29, 1997, and WO 97/26333, published Jul. 24, 1997, each of which is incorporated herein by reference in its entirety).

In addition to these direct labeling devices, indirect labels comprising enzymes can also be used according to the present invention. Various types of enzyme-linked immunoassays are well known in the art, for example, enzyme-linked immunoassays using alkaline phosphatase, horseradish peroxidase, lysozyme, glucose-6-phosphate dehydrogenase, lactate dehydrogenase, or urease. These and other similar assays are well known in the art and are disclosed, e.g., in Engvall (1980, “Enzyme Immunoassay ELISA and EMIT,” in Methods in Enzymology, 70: 419-439) and in U.S. Pat. No. 4,857,453.

In certain embodiments, proteins can be labeled by metabolic labeling. Metabolic labeling occurs during in vitro incubation of the cells that express the protein in the presence of culture medium supplemented with a metabolic label, such as [³⁵S]-methionine or [³²P]-orthophosphate. In addition to metabolic (or biosynthetic) labeling with [³⁵S]-methionine, the invention further contemplates labeling with [¹⁴C]-amino acids and [³H]-amino acids (with the tritium substituted at non-labile positions).

Below is a description of exemplary ICOS-B7RP-1 inhibitors. Other inhibitors can be identified according to the methods described in Section 4.5 infra.

### 4.2.1 B7RP-1- AND ICOS-BINDING POLYPEPTIDES

The present invention encompasses the use of forms of polypeptides that bind to the ICOS or B7RP-1 proteins in the methods and compositions of the present invention. Such proteins include full length ICOS proteins (for example, expressed by a cell that is administered to a patient) or peptide fragments thereof which bind to the B7RP-1 ligand, as well as full length B7RP-1 proteins (for example, expressed by a cell that is administered to a patient) or peptide fragments thereof which bind to the ICOS receptor. Such ICOS and B7RP-1 proteins include dominant negative forms of ICOS and B7RP-1. As used herein, a dominant negative ICOS or B7RP-1 protein refers to a form of ICOS or B7RP-1 that binds to B7RP-1 or ICOS, respectively, and when administered to a patient at least partially sequesters B7RP-1 or ICOS, respectively, thereby inhibiting, at least in part, the endogenous ICOS/B7RP-1 interaction.

The amino acid sequences depicted in SEQ ID NO: 2 and SEQ ID NO: 4 represent full length human and murine ICOS proteins, respectively, available as the translation products of the cDNAs described in Genbank accession nos. AJ277832 and AJ250559, respectively. The amino acid sequences depicted in SEQ ID NO: 6 and SEQ ID NO: 8 represent full length human and murine B7RP-1 proteins, respectively, available as the translation products of the cDNAs described in Genbank accession nos. AF216749 and NM_{—}015790, respectively.

Human ICOS is a 198 amino acid protein (SEQ ID NO: 2). The nucleotide sequence for human ICOS open reading frame is set forth in SEQ ID NO: 1. Mouse ICOS, encodes two transcripts, a shorter 2.1 Kb form and a longer 3.3 Kb form, that are overexpressed in CD3/TCR-activated Th2 cells. The mouse transcripts differ only in their 3′-untranslated region. The open reading frame (SEQ ID NO: 3) of both mouse transcripts encodes a predicted 200 amino acid, 22.7 kDa protein (SEQ ID NO: 4). The human ICOS protein and the corresponding mouse ICOS protein are Ig superfamily members, which share 69% identity over their full-length amino acid sequences.

The predicted human ICOS and mouse ICOS proteins share homology to both human and murine CD28 and CTLA-4. The human ICOS sequence shares 33% identity with hCD28 and 26% identity with hCTLA-4. The murine orthologue shares 36.5% identity with mCD28 and 38.5% identity with mCTLA-4. Examination of the amino acid sequence of mouse ICOS and of human ICOS revealed 4 conserved cysteine residues (amino acid residues 42, 63, 83, and 137 of SEQ ID NO: 4 and amino acid residues 41, 62, 82, and 135 of SEQ ID NO: 2).

Preferred in the present methods ane compositions are soluble B7RP-1 and ICOS polypeptides. Such polypeptides generally lack a transmembrane domain and an intracellular domain.

The use of the entire ICOS extracellular domain, or a B7RP-1 binding portion thereof, is contemplated in the present methods an compositions. The use of such polypeptides is desirable in the present methods. Exemplary ICOS polypeptides for this purpose are polypeptides comprising an ICOS fragment consisting essentially of amino acids 21-138 of SEQ ID NO: 2 (representing the extracellular domain of ICOS) and amino acids 26-132 of SEQ ID NO: 2 (representing the immunoglobulin homology domain of ICOS).

Fragments of ICOS or B7RP-1 that are useful in the methods and compositions present invention may contain deletions, additions or substitutions of amino acid residues within the amino acid sequence encoded by an ICOS or B7RP-1 gene. Preferably mutations result in a silent change, thus producing a functionally equivalent ICOS or B7RP-1 gene product.

An ICOS or B7RP-1 polypeptide sequence preferably comprises an amino acid sequence that exhibits at least about 65% sequence similarity to human ICOS or B7RP-1, more preferably exhibits at least 70% sequence similarity to human ICOS or B7RP-1, yet more preferably exhibits at least about 75% sequence similarity human ICOS or B7RP-1. In other embodiments, the ICOS or B7RP-1 polypeptide sequence preferably comprises an amino acid sequence that exhibits at least 85% sequence similarity to human ICOS or B7RP-1, yet more preferably exhibits at least 90% sequence similarity to human ICOS or B7RP-1, and most preferably exhibits at least about 95% sequence similarity to human ICOS or B7RP-1. In one embodiment, such a polypeptide sequence comprises all or a portion of the murine ICOS or B7RP-1 sequence, respectively.

In other embodiment, the ICOS or B7RP-1 polypeptide sequence preferably comprises an amino acid sequence that exhibits at least about 65% sequence identity to murine ICOS or B7RP-1, more preferably exhibits at least 70% sequence identity to murine ICOS or B7RP-1, yet more preferably exhibits at least about 75% sequence identity to murine ICOS or B7RP-1. In other embodiments, the ICOS or B7RP-1 polypeptide sequence preferably comprises an amino acid sequence that exhibits at least 85% sequence identity to murine ICOS or B7RP-1, yet more preferably exhibits at least 90% sequence identity to murine ICOS or B7RP-1, and most preferably exhibits at least about 95% sequence identity to murine ICOS or B7RP-1. In one embodiment, such a polypeptide sequence comprises a portion of murine ICOS that binds to the human B7RP-1 extracellular domain, or a portion of murine B7RP-1 that binds to the human ICOS extracellular domain, respectively.

The determination of percent identity between two sequences can be accomplished using a mathematical algorithm. A preferred, non-limiting example of a mathematical algorithm utilized for the comparison of two sequences is the algorithm of Karlin and Altschul (1990) Proc Natl Acad Sci. 87:2264-2268, modified as in Karlin and Altschul (1993) Proc Natl Acad Sci. 90:5873-5877. Such an algorithm is incorporated into the NBLAST and XBLAST programs of Altschul et al. (1990) J. Mol. Biol. 215:403-410. BLAST nucleotide searches can be performed with the NBLAST program, score=100, wordlength=12 to obtain nucleotide sequences homologous to a nucleic acid molecules of the invention. BLAST protein searches can be performed with the XBLAST program, score=50, wordlength=3 to obtain amino acid sequences homologous to a protein molecules of the invention. To obtain gapped alignments for comparison purposes, Gapped BLAST can be utilized as described in Altschul et al. (1997) Nucleic Acids Res. 25:3389-3402. Alternatively, PSI-Blast can be used to perform an iterated search which detects distant relationships between molecules (Id.). When utilizing BLAST, Gapped BLAST, and PSI-Blast programs, the default parameters of the respective programs (e.g., XBLAST and NBLAST) can be used.

Another preferred, non-limiting example of a mathematical algorithm utilized for the comparison of sequences is the algorithm of Myers and Miller, (1988) CABIOS 4:11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Additional algorithms for sequence analysis are known in the art and include ADVANCE and ADAM as described in Torellis and Robotti (1994) Comput. Appl. Biosci., 10:3-5; and FASTA described in Pearson and Lipman (1988) 85:2444-8. Within FASTA, ktup is a control option that sets the sensitivity and speed of the search. If ktup=2, similar regions in the two sequences being compared are found by looking at pairs of aligned residues; if ktup=1, single aligned amino acids are examined. ktup can be set to 2 or 1 for protein sequences, or from 1 to 6 for DNA sequences. The default if ktup is not specified is 2 for proteins and 6 for DNA. For a further description of FASTA parameters, see http://bioweb.pasteur.fr/docs/man/man/fasta.1.html#sect2.

The percent identity between two sequences can be determined using techniques similar to those described above, with or without allowing gaps. In calculating percent identity, only exact matches are counted. However, conservative substitutions should be considered in evaluating sequences that have a low percent identity with the ICOS or B7RP-1 sequences disclosed herein.

In a specific embodiment, polypeptides comprising at least 10, 20, 30, 40, 50, 75, 100, or 200 amino acids of SEQ ID NO: 2 or 4 that bind to B7RP-1, or polypeptides comprising at least 10, 20, 30, 40, 50, 75, 100, or 200 amino acids of SEQ ID NO: 6 or 8 that bind to ICOS, are used in the present invention. In a preferred embodiment, such a polypeptide comprises all or a portion of the extracellular domain of SEQ ID NO: 2, 4, 6, or 8.

In addition to the foregoing fragments and derivatives of ICOS and B7RP-1, dominant negative forms of other ICOS- and B7RP-1 binding polypeptides, for example other ligands to which ICOS binds and receptors to which B7RP-1 binds, respectively, may be used. Additionally, other ICOS- and B7RP-1 binding polypeptides can be identified according to the methods described in Section 4.5 below.

### 4.2.1.1 FUSION PROTEINS

Also useful in the present methods and compositions also are fusion proteins comprising a portion of an ICOS-binding polypeptide or a B7RP-1-binding polypeptide sequence which binds to ICOS or B7RP-1, respectively, operatively associated to a heterologous component, e.g., a heterologous peptide. Heterologous components can include, but are not limited to sequences which facilitate isolation and purification of the fusion protein. Heterologous components can also include sequences which confer stability to the B7RP-1- or ICOS-binding polypeptides. Such fusion partners are well known to those of skill in the art.

The present invention encompasses the use of fusion proteins comprising an ICOS (e.g., SEQ ID NO: 2 or SEQ ID NO: 4) or B7RP-1 polypeptide (SEQ ID NO: 6 and SEQ ID NO: 8) and a heterologous polypeptide (i.e., an unrelated polypeptide or fragment thereof, preferably at least 10, at least 20, at least 30, at least 40, at least 50, at least 60, at least 70, at least 80, at least 90 or at least 100 amino acids of the polypeptide). The fusion can be direct, but may occur through linker sequences. The heterologous polypeptide may be fused to the N-terminus or C-terminus of an B7RP-1- or ICOS-binding polypeptide.

A fusion protein can comprise an B7RP-1- or ICOS-binding polypeptide fused to a heterologous signal sequence at its N-terminus. Various signal sequences are commercially available. Eukaryotic heterologous signal sequences include, but art not limited to, the secretory sequences of melittin and human placental alkaline phosphatase (Stratagene; La Jolla, Calif.). Prokaryotic heterologous signal sequences useful in the methods of the invention include, but are not limited to, the phoA secretory signal (Sambrook et al., eds., Molecular Cloning: A Laboratory Manual, 2nd ed., Cold Spring Harbor Laboratory, Cold Spring Harbor Laboratory Press, Cold Spring Harbor, N.Y., 1989) and the protein A secretory signal (Pharmacia Biotech; Piscataway, N.J.).

The B7RP-1- or ICOS-binding protein or fragment thereof can be fused to tag sequences, e.g., a hexa-histidine peptide, such as the tag provided in a pQE vector (QIAGEN, Inc., Chatsworth, Calif., 91311), among others, many of which are commercially available for use in the methods of the invention. As described in Gentz et al., 1989, Proc. Natl. Acad. Sci. USA, 86:821-824, for instance, hexa-histidine provides for convenient purification of the fusion protein. Other examples of peptide tags are the hemagglutinin “HA” tag, which corresponds to an epitope derived from the influenza hemagglutinin protein (Wilson et al., 1984, Cell, 37:767) and the “flag” tag (Knappik et al., 1994, Biotechniques, 17(4):754-761). These tags are especially useful for purification of recombinantly produced polypeptides of the invention.

Any fusion protein may be readily purified by utilizing an antibody specific or selective for the fusion protein being expressed. For example, a system described by Janknecht et al. allows for the ready purification of non-denatured fusion proteins expressed in human cell lines (Janknecht et al., 1991, Proc. Natl. Acad. Sci. USA 88:8972). In this system, the gene of interest is subcloned into a vaccinia recombination plasmid such that the open reading frame of the gene is translationally fused to an amino-terminal tag consisting of six histidine residues. Extracts from cells infected with recombinant vaccinia virus are loaded onto Ni²⁺.nitriloacetic acid-agarose columns and histidine-tagged proteins are selectively eluted with imidazole-containing buffers.

An affinity label can also be fused at its amino terminal to the carboxyl terminal of the B7RP-1- or ICOS-binding protein or fragment thereof for use in the methods of the invention. The precise site at which the fusion is made in the carboxyl terminal is not critical. The optimal site can be determined by routine experimentation. An affinity label can also be fused at its carboxyl terminal to the amino terminal of the B7RP-1- or ICOS-binding polypeptide for use in the methods of the invention.

A variety of affinity labels known in the art may be used, such as, but not limited to, the immunoglobulin constant regions (see also Petty, 1996, Metal-chelate affinity chromatography, in Current Protocols in Molecular Biology, Vol. 2, Ed. Ausubel et al., Greene Publish. Assoc. & Wiley Interscience), glutathione S-transferase (GST; Smith, 1993, Methods Mol. Cell Bio. 4:220-229), the E. coli maltose binding protein (Guan et al., 1987, Gene 67:21-30), and various cellulose binding domains (U.S. Pat. Nos. 5,496,934; 5,202,247; 5,137,819; Tomme et al., 1994, Protein Eng. 7:117-123), etc. Other affinity labels are recognized by specific binding partners and thus facilitate isolation by affinity binding to the binding partner which can be immobilized onto a solid support. Some affinity labels may afford the B7RP-1- or ICOS-binding polypeptide novel structural properties, such as the ability to form multimers. These affinity labels are usually derived from proteins that normally exist as homopolymers. Affinity labels such as the extracellular domains of CD8 (Shiue et al., 1988, J. Exp. Med. 168:1993-2005), or CD28 (Lee et al., 1990, J. Immunol. 145:344-352), or fragments of the immunoglobulin molecule containing sites for interchain disulfide bonds, could lead to the formation of multimers.

As will be appreciated by those skilled in the art, many methods can be used to obtain the coding region of the above-mentioned affinity labels, including but not limited to, DNA cloning, DNA amplification, and synthetic methods. Some of the affinity labels and reagents for their detection and isolation are available commercially.

A preferred affinity label is a non-variable portion of the immunoglobulin molecule. Typically, such portions comprise at least a functionally operative CH2 and CH3 domain of the constant region of an immunoglobulin heavy chain. Fusions are also made using the carboxyl terminus of the Fc portion of a constant domain, or a region immediately amino-terminal to the CH1 of the heavy or light chain. Suitable immunoglobulin-based affinity label may be obtained from IgG-1, −2, −3, or −4 subtypes, IgA, IgE, IgD, or IgM, but preferably IgG1. Preferably, a human immunoglobulin is used when the B7RP-1- or ICOS-binding polypeptide is intended for in vivo use for humans. Many DNA encoding immunoglobulin ligh or heavy chain constant regions are known or readily available from cDNA libraries. See, for example, Adams et al., Biochemistry, 1980, 19:2711-2719; Gough et al., 1980, Biochemistry, 19:2702-2710; Dolby et al., 1980, Proc. Natl. Acad. Sci. U.S.A., 77:6027-6031; Rice et al., 1982, Proc. Natl. Acad. Sci. U.S.A., 79:7862-7865; Falkner et al., 1982, Nature, 298:286-288; and Morrison et al., 1984, Ann. Rev. Immunol, 2:239-256. Because many immunological reagents and labeling systems are available for the detection of immunoglobulins, the B7RP-1- or ICOS-binding polypeptide-Ig fusion protein can readily be detected and quantified by a variety of immunological techniques known in the art, such as the use of enzyme-linked immunosorbent assay (ELISA), immunoprecipitation, fluorescence activated cell sorting (FACS), etc. Similarly, if the affinity label is an epitope with readily available antibodies, such reagents can be used with the techniques mentioned above to detect, quantitate, and isolate the B7RP-1- or ICOS-binding polypeptide containing the affinity label. In many instances, there is no need to develop specific or selective antibodies to the B7RP-1- or ICOS-binding polypeptide for the purposes of purification.

A fusion protein can comprise an B7RP-1- or ICOS-binding polypeptide fused to the Fc domain of an immunoglobulin molecule or a fragment thereof for use in the methods of the invention. A fusion protein can also comprise an B7RP-1- or ICOS-binding polypeptide fused to the CH2 and/or CH3 region of the Fc domain of an immunoglobulin molecule. Furthermore, a fusion protein can comprise an B7RP-1- or ICOS-binding polypeptide fused to the CH2, CH3, and hinge regions of the Fe domain of an immunoglobulin molecule (see Bowen et al., 1996, J. Immunol. 156:442-49). This hinge region contains three cysteine residues which are normally involved in disulfide bonding with other cysteines in the Ig molecule. Since none of the cysteines are required for the peptide to function as a tag, one or more of these cysteine residues may optionally be substituted by another amino acid residue, such as for example, serine.

Various leader sequences known in the art can be used for the efficient secretion of the B7RP-1- or ICOS-binding polypeptide from bacterial and mammalian cells (von Heijne, 1985, J. Mol. Biol. 184:99-105). Leader peptides are selected based on the intended host cell, and may include bacterial, yeast, viral, animal, and mammalian sequences. For example, the herpes virus glycoprotein D leader peptide is suitable for use in a variety of mammalian cells. A preferred leader peptide for use in mammalian cells can be obtained from the V-J2-C region of the mouse immunoglobulin kappa chain (Bernard et al., 1981, Proc. Natl. Acad. Sci. 78:5812-5816). Preferred leader sequences for targeting ICOS- or B7RP-1-binding polypeptide expression in bacterial cells include, but are not limited to, the leader sequences of the E. coli proteins OmpA (Hobom et al., 1995, Dev. Biol. Stand. 84:255-262), Pho A (Oka et al., 1985, Proc. Natl. Acad. Sci 82:7212-16), OmpT (Johnson et al., 1996, Protein Expression 7:104-113), LamB and OmpF (Hoffman & Wright, 1985, Proc. Natl. Acad. Sci. USA 82:5107-5111), β-lactamase (Kadonaga et al., 1984, J. Biol. Chem. 259:2149-54), enterotoxins (Morioka-Fujimoto et al., 1991, J. Biol. Chem. 266:1728-32), and the Staphylococcus aureus protein A (Abrahmsen et al., 1986, Nucleic Acids Res. 14:7487-7500), and the B. subtilis endoglucanase (Lo et al., Appl. Environ. Microbiol. 54:2287-2292), as well as artificial and synthetic signal sequences (Maclntyre et al., 1990, Mol. Gen. Genet. 221:466-74; Kaiser et al., 1987, Science, 235:312-317).

Fusion proteins can be produced by standard recombinant DNA techniques or by protein synthetic techniques, e.g., by use of a peptide synthesizer. For example, a nucleic acid molecule encoding a fusion protein can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, e.g., Current Protocols in Molecular Biology, Ausubel et al., eds., John Wiley & Sons, 1992).

The nucleotide sequence coding for a fusion protein can be inserted into an appropriate expression vector, i.e., a vector which contains the necessary elements for the transcription and translation of the inserted protein-coding sequence. The expression of a fusion protein may be regulated by a constitutive, inducible or tissue-specific or -selective promoter. It will be understood by the skilled artisan that fusion proteins, which can facilitate solubility and/or expression, and can increase the in vivo half-life of the B7RP-1- or ICOS-binding polypeptide and thus are useful in the methods of the invention. The B7RP-1- or ICOS-binding polypeptides or peptide fragments thereof, or fusion proteins can be used in any assay that detects or measures B7RP-1- or ICOS-binding polypeptides or in the calibration and standardization of such assay.

The methods of invention encompass the use of B7RP-1- or ICOS-binding polypeptides or peptide fragments thereof, which may be produced by recombinant DNA technology using techniques well known in the art. Thus, methods for preparing the B7RP-1- or ICOS-binding polypeptides and peptides of the invention by expressing nucleic acid containing B7RP-1- or ICOS-binding gene sequences are described herein. Methods which are well known to those skilled in the art can be used to construct expression vectors containing, e.g., ICOS polypeptide coding sequences (including but not limited to nucleic acids encoding all or a B7RP-1-binding portion of ICOS) or B7RP-1 polypeptide coding sequences (including but not limited to nucleic acids encoding all or an ICOS-binding portion of B7RP-1) and appropriate transcriptional and translational control signals. These methods include, for example, in vitro recombinant DNA techniques, synthetic techniques, and in vivo genetic recombination. See, for example, the techniques described in Sambrook et al., 1989, supra, and Ausubel et al., 1989, supra. Alternatively, RNA capable of encoding B7RP-1- or ICOS-binding polypeptide sequences may be chemically synthesized using, for example, synthesizers (see e.g., the techniques described in Oligonucleotide Synthesis, 1984, Gait, M. J. ed., IRL Press, Oxford).

### 4.2.1.2 EXPRESSION SYSTEMS

A variety of host-expression vector systems may be utilized to express the B7RP-1- or ICOS-binding polypeptide coding sequences for use in the methods of the invention. Such host-expression systems represent vehicles by which the coding sequences of interest may be produced and subsequently purified, but also represent cells which may, when transformed or transfected with the appropriate nucleotide coding sequences, exhibit the B7RP-1- or ICOS-binding polypeptide of the invention in situ. These include but are not limited to microorganisms such as bacteria (e.g., E. coli, B. subtilis) transformed with recombinant bacteriophage DNA, plasmid DNA or cosmid DNA expression vectors containing B7RP-1- or ICOS-binding polypeptide coding sequences; yeast (e.g., Saccharomyces, Pichia) transformed with recombinant yeast expression vectors containing the B7RP-1- or ICOS-binding polypeptide coding sequences; insect cell systems infected with recombinant virus expression vectors (e.g., baculovirus) containing the B7RP-1- or ICOS-binding polypeptide coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing B7RP-1- or ICOS-binding polypeptide coding sequences; or mammalian cell systems (e.g., COS, CHO, BHK, 293, 3T3) harboring recombinant expression constructs containing promoters derived from the genome of mammalian cells (e.g., metallothionein promoter) or from mammalian viruses (e.g., the adenovirus late promoter; the vaccinia virus 7.5K promoter).

In bacterial systems, a number of expression vectors may be advantageously selected depending upon the use intended for the B7RP-1- or ICOS-binding polypeptide being expressed. For example, when a large quantity of such a protein is to be produced, for the generation of pharmaceutical compositions of B7RP-1- or ICOS-binding protein or for raising antibodies to ICOS or B7RP-1 protein, vectors which direct the expression of high levels of fusion protein products that are readily purified may be desirable. Such vectors include, but are not limited, to the E. coli expression vector pUR278 (Ruther et al., 1983, EMBO J. 2:1791), in which the ICOS or B7RP-1 polypeptide coding sequence may be ligated individually into the vector in frame with the lac Z coding region so that a fusion protein is produced; pIN vectors (Inouye & Inouye, 1985, Nucleic Acids Res. 13:3101; Van Heeke & Schuster, 1989, J. Biol. Chem. 264:5503); and the like. pGEX vectors may also be used to express foreign polypeptides as fusion proteins with glutathione S-transferase (GST). In general, such fusion proteins are soluble and can easily be purified from lysed cells by adsorption and binding to a matrix glutathione-agarose beads followed by elution in the presence of free glutathione. The pGEX vectors are designed to include, e.g.,thrombin or factor Xa protease cleavage sites so that the cloned target polypeptide can be released from the GST moiety.

In an insect system, Autographa californica nuclear polyhedrosis virus (AcNPV) is used as a vector to express foreign genes. The virus grows in Spodoptera frugiperda cells. The B7RP-1- or ICOS-binding polypeptide coding sequence may be cloned individually into non-essential regions (for example the polyhedrin gene) of the virus and placed under control of an AcNPV promoter (for example the polyhedrin promoter). Successful insertion of B7RP-1- or ICOS-binding polypeptide coding sequence will result in inactivation of the polyhedrin gene and production of non-occluded recombinant virus (i.e., virus lacking the proteinaceous coat coded for by the polyhedrin gene). These recombinant viruses are then used to infect Spodoptera frugiperda cells in which the inserted gene is expressed (e.g., see Smith et al., 1983, J. Virol. 46:584; Smith, U.S. Pat. No. 4,215,051).

In mammalian host cells, a number of viral-based expression systems may be utilized. In cases where an adenovirus is used as an expression vector, the B7RP-1- or ICOS-binding polypeptide coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing ICOS or B7RP-1 polypeptide in infected hosts. (See, e.g., Logan & Shenk, 1984, Proc. Natl. Acad. Sci. USA 81:3655). Specific initiation signals may also be required for efficient translation of inserted B7RP-1- or ICOS-binding polypeptide coding sequences. These signals include the ATG initiation codon and adjacent sequences. In cases where an entire B7RP-1- or ICOS-binding polypeptide gene, including its own initiation codon and adjacent sequences, is inserted into the appropriate expression vector, no additional translational control signals may be needed. However, in cases where only a portion of the B7RP-1- or ICOS-binding polypeptide coding sequence is inserted, exogenous translational control signals, including, perhaps, the ATG initiation codon, must be provided. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (See Bittner et al., 1987, Methods in Enzymol. 153:516).

In addition, a host cell strain may be chosen which modulates the expression of the inserted sequences, or modifies and processes the polypeptide in the specific fashion desired. Such modifications (e.g., glycosylation) and processing (e.g., cleavage) of protein products may be important for the function of the protein. Different host cells have characteristic and specific mechanisms for the post-transla- tional processing and modification of proteins and polypeptides. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the polypeptide may be used. Such mammalian host cells include but are not limited to CHO, VERO, BHK, HeLa, COS, MDCK, 293, 3T3, W138.

For long-term, high-yield production of recombinant proteins, stable expression is preferred. For example, cell lines which stably express the B7RP-1- or ICOS-binding polypeptide may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the B7RP-1- or ICOS-binding polypeptide. Such engineered cell lines may be particularly useful in screening and evaluation of compounds that affect the endogenous activity of the ICOS or B7RP-1 polypeptide.

A number of selection systems may be used, including but not limited to the herpes simplex virus thymidine kinase (Wigler et al., 1977, Cell 11:223), hypoxanthine-guanine phosphoribosyltransferase (Szybalska & Szybalski, 1962, Proc. Natl. Acad. Sci. USA 48:2026), and adenine phosphoribosyltransferase (Lowy et al., 1980, Cell 22:817) genes can be employed in tk⁻, hgprt⁻ or aprt⁻ cells, respectively. Also, antimetabolite resistance can be used as the basis of selection for the following genes: dhfr, which confers resistance to methotrexate (Wigler et al., 1980, Proc Natl. Acad. Sci. USA 77:3567; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Colberre-Garapin et al., 1981, J. Mol. Biol. 150: 1); and hygro, which confers resistance to hygromycin (Santerre et al., 1984, Gene 30:147).

### 4.2.2 ANTIBODIES

The methods of the present invention encompass the use of antibodies or fragments thereof capable of specifically or selectively recognizing one or more ICOS or B7RP-1 polypeptide epitopes or epitopes of conserved variants or peptide fragments of the ICOS or B7RP-1 polypeptides. Such antibodies may include, but are not limited to, polyclonal antibodies, monoclonal antibodies (mAbs), humanized or chimeric antibodies, single chain antibodies, Fab fragments, F(ab′)₂ fragments, Fv fragments, fragments produced by a Fab expression library, anti-idiotypic (anti-Id) antibodies, and epitope-binding fragments of any of the above. In a preferred embodiment, the anti-ICOS or anti-B7RP-1 antibody used in the present methods binds to the ICOS or B7RP-1 extracellular domain. In a most preferred embodiment, such an antibody blocks the interaction between ICOS and BR7P-1 without inducing signaling by the ICOS-BR7-1 pathway.

Described herein are methods for the production of antibodies or fragments thereof. Any of such antibodies or fragments thereof may be produced by standard immunological methods or by recombinant expression of nucleic acid molecules encoding the antibody or fragments thereof in an appropriate host organism.

For the production of antibodies against an ICOS or B7RP-1 polypeptide, various host animals may be immunized by injection with an ICOS or B7RP-1 polypeptide or peptide. Such host animals may include but are not limited to rabbits, mice, and rats, to name but a few. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum.

Polyclonal antibodies are heterogeneous populations of antibody molecules derived from the sera of animals immunized with an antigen, such as an ICOS or B7RP-1 polypeptide, or an antigenic functional derivative thereof. For the production of polyclonal antibodies, host animals such as those described above, may be immunized by injection with ICOS or B7RP-1 polypeptide supplemented with adjuvants as also described above.

Monoclonal antibodies, which are homogeneous populations of antibodies to a particular antigen, may be obtained by any technique which provides for the production of antibody molecules by continuous cell lines in culture. These include, but are not limited to, the hybridoma technique of Kohler and Milstein, (1975, Nature 256:495; and U.S. Pat. No. 4,376,110), the human B-cell hybridoma technique (Kosbor et al., 1983, Immunology Today 4:72; Cole et al., 1983, Proc. Natl. Acad. Sci. USA 80:2026), and the EBV-hybridoma technique (Cole et al., 1985, Monoclonal Antibodies And Cancer Therapy, Alan R. Liss, Inc., pp. 77). Such antibodies may be of any immunoglobulin class including IgG, IgM, IgE, IgA, IgD and any subclass thereof. The hybridoma producing the mAb of this invention may be cultivated in vitro or in vivo. Production of high titers of mAbs in vivo makes this the presently preferred method of production.

Techniques developed for the production of “chimeric antibodies” (Morrison et al., 1984, Proc. Natl. Acad. Sci., 81, 6851-6855; Neuberger et al., 1984, Nature 312, 604-608; Takeda et al., 1985, Nature 314, 452-454) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable region derived from a murine mAb and a human immunoglobulin constant region. (See, e.g., Cabilly et al., U.S. Pat. No. 4,816,567; and Boss et al., U.S. Pat. No. 5,816,397). The invention thus contemplates chimeric antibodies that are specific or selective for an ICOS or B7RP-1 polypeptide.

Examples of techniques that have been developed for the production of humanized antibodies are known in the art. (See, e.g., Queen, U.S. Pat. No. 5,585,089 and Winter, U.S. Pat. No. 5,225,539.) An immunoglobulin B7RP-1 or heavy chain variable region consists of a “framework” region interrupted by three hypervariable regions, referred to as complementarity-determining regions (CDRs). The extent of the framework region and CDRs have been precisely defined (see, “Sequences of Proteins of Immunological Interest”, Kabat, E. et al., U.S. Department of Health and Human Services (1983). Briefly, humanized antibodies are antibody molecules from non-human species having one or more CDRs from the non-human species and framework regions from a human immunoglobulin molecule. The invention includes the use of humanized antibodies that are specific or selective for an ICOS or B7RP-1 polypeptide in the methods and compositions of the invention.

Completely human ICOS or B7RP-1 antibodies are particularly desirable for therapeutic treatment of human patients. Such antibodies can be produced, for example, using transgenic mice which are incapable of expressing endogenous immunoglobulin heavy and B7RP-1 chains genes, but which can express human heavy and B7RP-1 chain genes. The transgenic mice are immunized in the normal fashion with a selected antigen, e.g., all or a portion of an ICOS or B7RP-1 protein. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g., U.S. Pat. No. 5,625,126; U.S. Pat. No. 5,633,425; U.S. Pat. No. 5,569,825; U.S. Pat. No. 5,661,016; and U.S. Pat. No. 5,545,806. In addition, companies such as Abgenix, Inc. (Freemont, Calif.), can be engaged to provide human antibodies directed against a selected ICOS or B7RP-1 antigen using technology similar to that described above.

Completely human antibodies which recognize a selected epitope can be generated using a technique referred to as “guided selection.” In this approach a selected non-human monoclonal antibody, e.g., a murine antibody, is used to guide the selection of a completely human antibody recognizing the same epitope (Jespers et al., 1994, Bio/technology 12:899-903).

The methods of the invention encompasses the use of an antibody or derivative thereof comprising a heavy or B7RP-1 chain variable domain, said variable domain comprising (a) a set of three complementarity-determining regions (CDRs), in which said set of CDRs are from a monoclonal antibody to an ICOS or B7RP-1 polypeptide, most preferably to the ICOS or B7RP-1 extracellular domain, and (b) a set of four framework regions, in which said set of framework regions differs from the set of framework regions in the monoclonal antibody, and in which said antibody or derivative thereof immunospecifically binds to the ICOS or B7RP-1 polypeptide. Preferably, the set of framework regions is from a human monoclonal antibody, e.g., a human monoclonal antibody that does not bind the polypeptide encoded for by the ICOS or B7RP-1 gene sequence.

Phage display technology can be used to increase the affinity of an antibody to an ICOS or B7RP-1 polypeptide. This technique would be useful in obtaining high affinity antibodies to an ICOS or B7RP-1 polypeptide that could be used in the combinatorial methods of the invention. The technology, referred to as affinity maturation, employs mutagenesis or CDR walking and re-selection using the ICOS or B7RP-1 polypeptide antigen to identify antibodies that bind with higher affinity to the antigen when compared with the initial or parental antibody (see, e.g., Glaser et al., 1992, J. Immunology 149:3903). Mutagenizing entire codons rather than single nucleotides results in a semi-randomized repertoire of amino acid mutations. Libraries can be constructed consisting of a pool of variant clones each of which differs by a single amino acid alteration in a single CDR and which contain variants representing each possible amino acid substitution for each CDR residue. Mutants with increased binding affinity for the antigen can be screened by contacting the immobilized mutants with labeled antigen. Any screening method known in the art can be used to identify mutant antibodies with increased avidity to the antigen (e.g., ELISA) (See Wu et al., 1998, Proc Natl. Acad Sci. USA 95:6037; Yelton et al., 1995, J. Immunology 155:1994). CDR walking which randomizes the light chain is also possible (See Schier et al., 1996, J. Mol. Bio. 263:551).

Alternatively, techniques described for the production of single chain antibodies (U.S. Pat. No. 4,946,778; Bird, 1988, Science 242:423; Huston et al., 1988, Proc. Natl. Acad. Sci. USA 85:5879; and Ward et al., 1989, Nature 334:544) can be adapted to produce single chain antibodies against ICOS or B7RP-1 polypeptides. Single chain antibodies are formed by linking the heavy and B7RP-1 chain fragments of the Fv region via an amino acid bridge, resulting in a single chain polypeptide. Techniques for the assembly of functional Fv fragments in E. coli may also be used (Skerra et al., 1988, Science 242:1038).

The methods of the invention include using an antibody to an ICOS or B7RP-1 polypeptide, peptide or other derivative, or analog thereof that is a bispecific antibody (see generally, e.g., Fanger and Drakeman, 1995, Drug News and Perspectives 8:133-137). Such a bispecific antibody is genetically engineered to recognize both (1) an epitope and (2) one of a variety of “trigger” molecules, e.g., Fc receptors on myeloid cells, and CD3 and CD2 on T cells, that have been identified as being able to cause a cytotoxic T-cell to destroy a particular target. Such bispecific antibodies can be prepared either by chemical conjugation, hybridoma, or recombinant molecular biology techniques known to the skilled artisan.

Antibody fragments which recognize specific epitopes may be generated by known techniques. For example, such fragments include but are not limited to: the F(ab′)₂ fragments which can be produced by pepsin digestion of the antibody molecule and the Fab fragments which can be generated by reducing the disulfide bridges of the F(ab′)₂ fragments. Alternatively, Fab expression libraries may be constructed (Huse et al., 1989, Science 246:1275-1281) to allow rapid and easy identification of monoclonal Fab fragments with the desired specificity.

In a specific embodiment, monoclonal antibody 12A8, a non-depleting, rat-anti-mouse antibody that blocks ICOS is used (isotype IgG2b, Millennium Pharmaceuticals; Rottman et al., 2001, Nature Immunol. 2(7): 605-611; Özkaynak et al., Importance of ICOS-B7RP-1 co-stimulation in acute and chronic allograft rejection. Nature Immunol. 2, 591-596 (2001)). mAb 12A8 is a rat-anti-mouse ICOS, isotype IgG2b, that blocks binding of the ligand B7RP-1 to murine ICOS transfectant cells. The antibody has a half-life of approximately 14 h in vivo and, based upon flow cytometric analysis and immunohistology, does not deplete ICOS⁺ T cells from peripheral blood or tissues. In vivo treatment of mice with this antibody has been previously known to elicit a strong neutralizing anti-rat response, which begins by day 12 of treatment (Özkaynak E et al. Importance of ICOS-B7RP-1 co-stimulation in acute and chronic allograft rejection. Nature Immunol. 2, 591-596 (2001)).

In another embodiment of this type, monoclonal antibody 8F3, a rat-anti-mouse B7RP-1 antibody (isotype IgG2a, Millennium Pharmaceuticals) is used.

In another embodiment of this type, the blocking antibodies to ICOS and B7RP-1 disclosed in Wahl et al. (2002, J. Am. Soc. Nephrol. 13:1517-1526) are used.

It will be apparent to one of skill in the art that certain ICOS-B7RP-1 inhibitors may have both the properties of both an activator (e.g., agonist) and an inhibitor (e.g., an antagonist). The compounds listed hereinabove are not limited by theory of mechanism but are applicable to the present invention independently of their classification.

### 4.2.3 B7RP-1 OR ICOS ANTISENSE COMPOUNDS

The present invention encompasses the use of B7RP-1 and ICOS antisense nucleic acid molecules, i.e., molecules which are complementary to a sense nucleic acid encoding B7RP-1 and ICOS, respectively, e.g., complementary to the coding strand of a double-stranded cDNA molecule or complementary to an mRNA sequence as ICOS-B7RP-1 inhibitors. Accordingly, an antisense nucleic acid can hydrogen bond to a sense nucleic acid. The antisense nucleic acid can be complementary to an entire coding strand, or to only a portion thereof, e.g., all or part of the protein coding region (or open reading frame) of ICOS or B7RP-1. An antisense nucleic acid molecule can be antisense to all or part of a non-coding region of the coding strand of a nucleotide sequence encoding ICOS or B7RP-1. The non-coding regions (“5′ and 3′ untranslated regions”) are the 5′ and 3′ sequences which flank the coding region and are not translated into amino acids.

An antisense oligonucleotide can be, for example, about 5, 10, 15, 20, 25, 30, 35, 40, 45 or 50 nucleotides or more in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to an ICOS or B7RP-1 nucleic acid).

The antisense nucleic acid molecules of the invention are typically administered to a subject or generated in situ such that they hybridize with or bind to cellular mRNA and/or genomic DNA encoding ICOS or B7RP-1 to thereby inhibit expression, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of ICOS or B7RP-1 antisense nucleic acid molecules includes direct injection at a target tissue site, for example into the circulation for ICOS antisense nucleic acids and into the cerebrospinal fluid in the case of B7RP-1 antisense nucleic acids. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

An ICOS or B7RP-1 antisense nucleic acid molecule can be an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

The invention also encompasses ribozymes. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haselhoff and Gerlach, (1988), Nature 334:585-591)) can be used to catalytically cleave ICOS or B7RP-1 mRNA transcripts to thereby inhibit translation of the ICOS or B7RP-1 protein encoded by the mRNA. A ribozyme having specificity for an ICOS or B7RP-1 nucleic acid molecule can be designed based upon the nucleotide sequence of the ICOS or B7RP-1 cDNAs disclosed herein. For example, a derivative of a Tetrahymena L-19 IVS RNA can be constructed in which the nucleotide sequence of the active site is complementary to the nucleotide sequence to be cleaved in a Cech et al. U.S. Pat. No. 4,987,071; and Cech et al. U.S. Pat. No. 5,116,742. Alternatively, an mRNA encoding a polypeptide of the invention can be used to select a catalytic RNA having a specific ribonuclease activity from a pool of RNA molecules. See, e.g., Bartel and Szostak (1993) Science 261:1411-1418.

The invention also encompasses nucleic acid molecules which form triple helical structures. For example, expression of ICOS or B7RP-1 can be inhibited by targeting nucleotide sequences complementary to the regulatory region of the gene encoding ICOS or B7RP-1, respectively (e.g., the promoter and/or enhancer), to form triple helical structures that prevent transcription of the gene in target cells. See generally Helene (1991) Anticancer Drug Des. 6(6):569-84; Helene (1992) Ann. N.Y. Acad. Sci. 660:27-36; and Maher (1992) Bioassays 14(12):807-15.

In various embodiments, the antisense nucleic acid molecules of the invention can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids (see Hyrup et al. (1996) Bioorganic & Medicinal Chemistry 4(1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996), supra; Perry-O'Keefe et al. (1996) Proc. Natl. Acad. Sci. USA 93: 14670-675.

In another embodiment, PNAs can be modified, e.g, to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras can be generated which may combine the advantageous properties of PNA and DNA. Such chimeras allow DNA recognition enzymes, e.g., RNAse H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup (1996), supra). The synthesis of PNA-DNA chimeras can be performed as described in Hyrup (1996), supra, and Finn et al. (1996) Nucleic Acids Res. 24(17):3357-63. For example, a DNA chain can be synthesized on a solid support using standard phosphoramidite coupling chemistry and modified nucleoside analogs. Compounds such as 5′-(4-methoxytrityl)amino-5′-deoxy-thymidine phosphoramidite can be used as a link between the PNA and the 5′ end of DNA (Mag et al. (1989) Nucleic Acids Res. 17:5973-88). PNA monomers are then coupled in a stepwise manner to produce a chimeric molecule with a 5′ PNA segment and a 3′ DNA segment (Finn et al. (1996) Nucleic Acids Res. 24(17):3357-63). Alternatively, chimeric molecules can be synthesized with a 5′ DNA segment and a 3′ PNA segment (Peterser et al. (1975) Bioorganic Med. Chem. Lett. 5:1119-11124).

In other embodiments, the ICOS or B7RP-1 antisense oligonucleotide may include other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; PCT Publication No. WO 88/09810) or the blood-brain barrier (see, e.g., PCT Publication No. WO 89/10134). In addition, the antisense oligonucleotides can be modified with hybridization-triggered cleavage agents (see, e.g., Krol et al. (1988) Bio/Techniques 6:958-976) or intercalating agents (see, e.g., Zon (1988) Pharm. Res. 5:539-549). To this end, the antisense oligonucleotide may be conjugated to another molecule, e.g., a peptide, hybridization triggered cross-linking agent, transport agent, hybridization-triggered cleavage agent, etc.

### 4.3 COMBINATION THERAPY

Described below are combinatorial methods and related compositions for treating or preventing inflammatory demyelinating disorders such as multiple sclerosis. The combinatorial methods of the invention involve the administration of at least two agents to a patient, the first of which is an ICOS-B7RP-1 inhibitor, and the second of which is a second therapeutic agent.

The combinatorial therapy methods of the present invention can result in a greater than additive effect, providing therapeutic benefits where neither the ICOS-B7RP-1 inhibitor or second therapeutic agent administered in an amount that is alone effective for treatment or prevention of an inflammatory demyelinating disorder.

In the present methods, the ICOS-B7RP-1 inhibitor and the second therapeutic agent can be administered concurrently or successively. As used herein, the ICOS-B7RP-1 inhibitor and the second therapeutic agent are said to be administered concurrently if they are administered to the patient on the same day, for example, simultaneously, or 1, 2, 3, 4, 5, 6, 7 or 8 hours apart. In contrast, the ICOS-B7RP-1 inhibitor and the second therapeutic agent are said to be administered successively if they are administered to the patient on the different days, for example, the ICOS-B7RP-1 inhibitor and the second therapeutic agent can be administered at a 1-day, 2-day or 3-day intervals. In the methods of the present invention, administration of the ICOS-B7RP-1 inhibitor can precede or follow administration of the second therapeutic agent.

As a non-limiting example, the ICOS-B7RP-1 inhibitor and second therapeutic agent can be administered concurrently for a period of time, followed by a second period of time in which the administration of the ICOS-B7RP-1 inhibitor and the second therapeutic agent is alternated.

The therapeutic regimens of the present invention can be practiced as long as the treatment or prevention of an inflammatory demyelinating disorder is required or desired.

Because of the potentially synergistic effects of administering a ICOS-B7RP-1 inhibitor and a second therapeutic agent, such agents can be administered in amounts that, if one or both of the agents is administered alone, is/are not effective for treating or preventing an inflammatory demyelinating disorder of interest such as multiple sclerosis.

With respect to multiple sclerosis, which is characterized by periods of disability (relapse) alternating with periods of recovery (remission), and eventually can result in chronic progressive multiple sclerosis, the combination therapy methods of the present invention can be administered during any of these periods, concurrently or in an alternating manner. A few non limiting embodiments of such modes of administration are described below. For example, the second therapeutic agent can administered concurrently with the ICOS-B7RP-1 inhibitor. Such concurrent administration can take place during a period of relapse in multiple sclerosis, during a period of disease remission, or during chronic progressive phase of the disease. Alternatively, the second therapeutic agent and the ICOS-B7RP-1 inhibitor are administered successively. In such methods of successive administration, the second therapeutic agent can be administered prior to administration of the ICOS-B7RP-1 inhibitor or after administration of the ICOS-B7RP-1 inhibitor. The ICOS-B7RP-1 inhibitor and the second therapeutic agent can be administered successively during the same phase of the disease, for example during remission, relapse or chronic progressive phase of multiple sclerosis in a patient. Alternatively, the ICOS-B7RP-1 inhibitor and the second therapeutic agent can be administered successively at different phases of the disease. For example, the ICOS-B7RP-1 inhibitor can be administered during a period of relapse and the second therapeutic agent can administered during a period of remission in the same patient, or vice versa.

Preferred second therapeutic agents that can be used in accordance with the combinatorial methods of the present invention include immunosuppressive agents and biological response modifiers, which are not mutually exclusive categories of second therapeutic agents. Exemplary immunosuppressive agents and biological response modifiers are described below in Sections 4.3.1 and 4.3.1, respectively.

### 4.3.1 IMMUNOSUPPRESSIVE AGENTS

As described herein, certain embodiments of the present invention encompasses the use of immunosuppressive agents in combination with an ICOS-B7RP-1 inhibitor to prevent or treat inflammation in the CNS. Any immunosuppressive agent known to those of skill in the art may be used. Such an immunosuppressive agent can be a drug or other small molecule, or a protein, including but not limited to an antibody. As used herein, the term “immunosuppressive agent” excludes ICOS-B7RP-1 inhibitors with immunosuppressive activity.

In certain specific embodiments of the invention, the immunosuppressive agent is cyclosporine, FK506, rapamycin, or prednisone.

In other embodiments, the immunosuppressive agent is a steroid, most preferably a corticosteroid.

In other embodiments, the immunosuppressive agent is an antibody, most preferably an anti-T cell antibody. In one embodiment, the antibody is an anti-CD154 antibody. In another embodiment, the antibody is an anti-CD3 antibody such as OKT3. In yet another embodiment, the antibody is an anti-interleukin-2 receptor antibody. Preparation of immunosuppressive antibodies that are suitable for the claimed methods and compositions can be carried out as described supra in Section 4.2.2.

In yet other embodiments, the immunosuppressive agent is a protein, for example a CTLA4-Ig fusion protein, a CD40-Ig fusion protein, or a CD28-Ig fusio protein.

In yet other embodiments, the immunosuppressive agent is an antibody, for example an anti-CTLA4-antibody, an anti-CD40 antibody, or an anti-CD28 antibody.

In yet other embodiments, the immunosuppressive agent is an antiproliferative agent, such as, but not limited to azathiopurine or mycophenolate moefitil.

In yet other embodiments, the immunosuppressive agent is a purine analog. In one embodiment, the purine analog is methotrexate. In another embodiment, the purine analog mercaptopurine.

### 4.3.2 BIOLOGICAL RESPONSE MODIFIERS

The present embodiment encompasses methods of treatment of demyelinating inflammatory disorders of the central nervous system comprising administering both an ICOS-B7RP-1 inhibitor. Such biological response modifiers are molecules that are capable of modulating the immune response of the patient to an ICOS-B7RP-1 inhibitor if administered concurrently with the ICOS-B7RP-1 inhibitor. The biological response modifiers of the invention include agent that promote a desired Th1 vs. Th2 ratio following treatment with an ICOS-B7RP-1 inhibitor, for example an interleukin such as interleukin 4, or an inhibitor of CCR1, RANTES, MCP-1, MIP-2, IL-1α, IL-1β, IL-6, IL-12p35, CD28, CTLA-4 or IFN-γ, such an antibody or antisense nucleic acid. Soluble versions of those proteins that are normally transmembrane proteins, such as CTLA-4 and CD28.

### 4.4 GENE THERAPY

In certain embodiments of the present invention, administration of an ICOS-B7RP-1 inhibitor or an immunosuppressive agent in the form of gene therapy is contemplated. Gene therapy refers to therapy performed by the administration to a subject of an expressed or expressible nucleic acid. In this embodiment of the invention, the nucleic acids produce their encoded protein that mediates a therapeutic or prophylactic effect.

### 4.4.1 NUCLEIC ACIDS ENCODING ICOS- AND B7RP-1-BINDING POLYPEPTIDES

The present invention provides nucleic acids encoding forms of the ICOS- and B7RP-1-binding polypeptides described in Section 4.2.1, spra, for use in expression and gene therapy vectors suitable for production or delivery, respectively, of such polypeptides, to a patient in need thereof.

Nucleic acids useful in the gene therapy methods of the present invention encode the minimal domain of a polypeptide such as ICOS that interacts with B7RP-1, or the minimal domain of a polypeptide such as B7RP-1 that interacts with ICOS. Such nucleic acids preferably encode soluble, including secreted, ICOS or B7RP-1 proteins that interfere with endogenous ICOS-B7RP-1 interactions in the patients to whom they are administered.

The present invention further encompasses the use of nucleic acids comprising a region of homology to a nucleic acid encoding the ICOS-binding domain of B7RP-1, or the B7RP-1-binding domain of ICOS, as long as such a nucleic acid encodes a polypeptide that can bind to ICOS or B7RP-1, respectively, and interfere with endogenous ICOS-B7RP-1 interactions in a patient to whom the polypeptide is administered. In various embodiments, the region of homology is characterized by at least 50%, at least 55%, at least 60%, at least 65%, at least 70%, at least 75%, at least 80%, at least 85%, at least 90%, at least 95% or at least 98% identity with nucleotides consisting essentially of the regions in the ICOS or B7RP-1 open reading frames encoding the extracellular domains of the proteins. Methods of determining sequence homology are described in Section 4.2.1 above.

The invention also encompasses the use of nucleic acids that (1) hybridize under stringent, moderate or low stringency hybridization conditions to a nucleic acid consisting essentially of the regions in the ICOS or B7RP-1 open reading frames encoding the extracellular domains of the proteins and (2) encode polypeptides which bind to B7RP-1 or ICOS, respectively. Preferably, such encoded polypeptides do not comprise a transmembrane domain.

By way of example and not limitation, procedures using such conditions of low stringency for regions of hybridization of over 90 nucleotides are as follows (see also Shilo and Weinberg, 1981, Proc. Natl. Acad. Sci. U.S.A. 78,:6789-6792). Filters containing DNA are pretreated for 6 hours at 40° C. in a solution containing 35% formamide, 5×SSC, 50 mM Tris-HCl (pH 7.5), 5 mM EDTA, 0.1% PVP, 0.1% Ficoll, 1% BSA, and 500 μg/ml denatured salmon sperm DNA. Hybridizations are carried out in the same solution with the following modifications: 0.02% PVP, 0.02% Ficoll, 0.2% BSA, 100 μg/ml salmon sperm DNA, 10% (wt/vol) dextran sulfate, and 5-20×10⁶ cpm ³²P-labeled probe is used. Filters are incubated in hybridization mixture for 18-20 h at 40° C., and then washed for 1.5 h at 55° C. in a solution containing 2×SSC, 25 mM Tris-HCI (pH 7.4), 5 mM EDTA, and 0.1% SDS. The wash solution is replaced with fresh solution and incubated an additional 1.5 h at 60° C. Filters are blotted dry and exposed for autoradiography. If necessary, filters are washed for a third time at 65-68° C. and re-exposed to film. Other conditions of low stringency which may be used are well known in the art (e.g., as employed for cross-species hybridizations).

Also, by way of example and not limitation, procedures using such conditions of high stringency for regions of hybridization of over 90 nucleotides are as follows. Prehybridization of filters containing DNA is carried out for 8 h to overnight at 65° C. in buffer composed of 6×SSC, 50 mM Tris-HCl (pH 7.5), 1 mM EDTA, 0.02% PVP, 0.02% Ficoll, 0.02% BSA, and 500 μg/ml denatured salmon sperm DNA. Filters are hybridized for 48 h at 65° C. in prehybridization mixture containing 100 μg/ml denatured salmon sperm DNA and 5-20×10⁶ cpm of ³²P-labeled probe. Washing of filters is done at 37° C. for 1 h in a solution containing 2×SSC, 0.01% PVP, 0.01% Ficoll, and 0.01% BSA. This is followed by a wash in 0.1×SSC at 50° C. for 45 min before autoradiography.

Other conditions of high stringency which may be used depend on the nature of the nucleic acid (e.g., length, GC content, etc.) and the purpose of the hybridization (detection, amplification, etc.) and are well known in the art. For example, stringent hybridization of a nucleic acid of approximately 15-40 bases to a complementary sequence in the polymerase chain reaction (PCR) is done under the following conditions: a salt concentration of 50 mM KCl, a buffer concentration of 10 mM Tris-HCl, a Mg²⁺ concentration of 1.5 mM, a pH of 7-7.5 and an annealing temperature of 55-60° C.

Selection of appropriate conditions for moderate stringencies is also well known in the art (see, e.g., Sambrook et al., 1989, Molecular Cloning, A Laboratory Manual, 2d Ed., Cold Spring Harbor Laboratory Press, Cold Spring Harbor, N.Y.; see also, Ausubel et al., eds., in the Current Protocols in Molecular Biology series of laboratory technique manuals, © 1987-1997, Current Protocols, © 1994-1997 John Wiley and Sons, Inc.).

The nucleic acids useful in the present methods may be made by any method known in the art. For example, if the nucleotide sequence of the protein is known, a nucleic acid encoding the antibody may be assembled from chemically synthesized oligonucleotides (e.g., as described in Kutmeier et al., 1994, BioTechniques 17:242), which, briefly, involves the synthesis of overlapping oligonucleotides containing portions of the sequence encoding the protein, annealing and ligating of those oligonucleotides, and then amplification of the ligated oligonucleotides by PCR.

Alternatively, a nucleic acid that is useful in the present methods may be generated from nucleic acid from a suitable source. If a clone containing a nucleic acid encoding a particular protein is not available, but the sequence of the protein molecule is known, a nucleic acid encoding the protein may be chemically synthesized or obtained from a suitable source (e.g., a cDNA library such as an antibody cDNA library or a cDNA library generated from, or nucleic acid, preferably poly A+ RNA, isolated from, any tissue or cells expressing the protein.

Further, a nucleic acid that is useful in the present methods may be manipulated using methods well known in the art for the manipulation of nucleotide sequences, e.g., recombinant DNA techniques, site directed mutagenesis, PCR, etc. (see, for example, the techniques described in Sambrook et al, 1990, Molecular Cloning, A Laboratory Manual, 2d Ed., Cold Spring Harbor Laboratory, Cold Spring Harbor, N.Y. and Ausubel et al., eds., 1998, Current Protocols in Molecular Biology, John Wiley & Sons, NY, which are both incorporated by reference herein in their entireties), to generate antibodies having a different amino acid sequence, for example to create amino acid substitutions, deletions, and/or insertions.

### 4.4.2 GENE THERAPY METHODS

Any of the methods for gene therapy available in the art can be used in the methods and compositions of the present invention. Exemplary methods are described below.

For general reviews of the methods of gene therapy, see, Goldspiel et al., 1993, Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; Morgan and Anderson, 1993, Ann. Rev. Biochem. 62:191-217; May, 1993, TIBTECH 1, 1(5): 155-215. Methods commonly known in the art of recombinant DNA technology which can be used are described in Ausubel et al. (eds.), Current Protocols in Molecular Biology, John Wiley & Sons, NY (1993); and Kriegler, Gene Transfer and Expression, A Laboratory Manual, Stockton Press, N.Y. (1990).

In a preferred aspect, the therapeutic comprises nucleic acid sequences encoding an ICOS-B7RP-1 inhibitor, said nucleic acid sequences being part of expression vectors that express the ICOS-B7RP-1 inhibitor in a suitable host. In particular, such nucleic acid sequences have promoters operably linked to the ICOS-B7RP-1 inhibitor coding region, said promoter being inducible or constitutive, and, optionally, tissue-specific. In another particular embodiment, nucleic acid molecules are used in which the ICOS-B7RP-1 inhibitor or immunosuppressive agent coding sequences and any other desired sequences are flanked by regions that promote homologous recombination at a desired site in the genome, thus providing for intrachromosomal expression of the ICOS-B7RP-1 inhibitor (Koller and Smithies, 1989, Proc. Natl. Acad. Sci. USA 86:8932-8935; Zijlstra et al., 1989, Nature 342:435-438.

Delivery of the nucleic acids into a patient may be either direct, in which case the patient is directly exposed to the nucleic acid or nucleic acid-carrying vectors, or indirect, in which case, cells are first transformed with the nucleic acids in vitro, then transplanted into the patient. These two approaches are known, respectively, as in vivo or ex vivo gene therapy.

In a specific embodiment, the nucleic acid sequences are directly administered in vivo, where it is expressed to produce the encoded product. This can be accomplished by any of numerous methods known in the art, for example by constructing them as part of an appropriate nucleic acid expression vector and administering the vector so that the nucleic acid sequences become intracellular. Gene therapy vectors can be administered by infection using defective or attenuated retrovirals or other viral vectors (see, e.g., U.S. Pat. No. 4,980,286); direct injection of naked DNA; use of microparticle bombardment (e.g., a gene gun; Biolistic, Dupont); coating with lipids or cell-surface receptors or transfecting agents; encapsulation in liposomes, microparticles, or microcapsules; administration in linkage to a peptide which is known to enter the nucleus; administration in linkage to a ligand subject to receptor-mediated endocytosis (see, e.g., Wu and Wu, 1987, J. Biol. Chem. 262:4429-4432) (which can be used to target cell types specifically expressing the receptors); etc. In another embodiment, nucleic acid-ligand complexes can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06 180; WO 92/22635; W)92/20316; W093/14188, and WO 93/20221). Alternatively, the nucleic acid can be introduced intracellularly and incorporated within host cell DNA for expression by homologous recombination (Koller and Smithies, 1989, Proc. Natl. Acad. Sci. USA 86:8932-8935; Zijlstra et al., 1989, Nature 342:435-438).

In a specific embodiment, viral vectors that contain nucleic acid sequences encoding an ICOS-B7RP-1 inhibitor are used. For example, a retroviral vector can be used (see Miller et al., 1993, Meth. Enzymol. 217:581-599). These retroviral vectors contain the components necessary for the correct packaging of the viral genome and integration into the host cell DNA. The nucleic acid sequences encoding the ICOS-B7RP-1 inhibitor to be used in gene therapy are cloned into one or more vectors, thereby facilitating delivery of the gene into a patient. More detail about retroviral vectors can be found in Boesen et al., 1994, Biotherapy 6:29 1-302, which describes the use of a retroviral vector to deliver the mdr 1 gene to hematopoietic stem cells in order to make the stem cells more resistant to chemotherapy. Other references illustrating the use of retroviral vectors in gene therapy are: Clowes et al., 1994, J. Clin. Invest. 93:644-65 1; Klein et al., 1994, Blood 83:1467-1473; Salmons and Gunzberg, 1993, Human Gene Therapy 4:129-141; and Grossman and Wilson, 1993, Curr. Opin. in Genetics and Devel. 3:110-114.

### 4.4.2.1 CELL THERAPY

One approach to gene therapy encompassed by the present methods and compositions involves transferring a gene, e.g., an ICOS-B7RP-1 inhibitor, to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a patient.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell mediated gene transfer, spheroplast fusion, etc. Numerous techniques are known in the art for the introduction of foreign genes into cells (see, e.g., Loeffler and Behr, 1993, Meth. Enzymol. 217:599-618; Cohen et al., 1993, Meth. Enzymol. 217:618-644; Cline, 1985, Pharmac. Ther. 29:69-92) and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

The resulting recombinant cells can be delivered to a patient by various methods known in the art. Recombinant blood cells (e.g., hematopoietic stem or progenitor cells) are preferably administered intravenously. The amount of cells envisioned for use depends on the desired effect, patient state, etc., and can be determined by one skilled in the art.

Cells into which a nucleic acid can be introduced for purposes of gene therapy are preferably T lymphocytes, B lymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood, fetal liver, etc.

In a preferred embodiment, such an ICOS nucleic acid is introduced into a T lymphocyte, preferably a T lymphocyte that expresses interleukin-10 at a high level, whether endogenously or recombinantly. Such a T-lymphocyte can then be used for gene therapy of a demyelinating inflammatory disorder, for example multiple sclerosis.

In a preferred embodiment, the cell used for gene therapy is autologous to the patient.

In an embodiment in which recombinant cells are used in gene therapy, nucleic acid sequences encoding an ICOS-B7RP-1 inhibitor are introduced into the cells such that they are expressible by the cells or their progeny, and the recombinant cells are then administered in vivo for therapeutic effect. In a specific embodiment, stem or progenitor cells are used. Any stem and/or progenitor cells which can be isolated and maintained in vitro can potentially be used in accordance with this embodiment of the present invention (see e.g. PCT Publication WO 94/08598; Stemple and Anderson, 1992, Cell 71:973-985; Rheinwald, 1980, Meth. Cell Bio. 21A:229; and Pittelkow and Scott, 1986, Mayo Clinic Proc. 61:771).

In a specific embodiment, the nucleic acid to be introduced for purposes of gene therapy comprises an inducible promoter operably linked to the coding region, such that expression of the nucleic acid is controllable by controlling the presence or absence of the appropriate inducer of transcription.

### 4.5 METHODS OF IDENTIFYING ICOS-B7RP-1 INHIBITORS

As disclosed herein, the present invention provides methods of treating or preventing inflammatory disorders, in particular inflammatory disorders of the central nervous system. Such methods entail administering to a patient in need of such treatment an ICOS-B7RP-1 inhibitor, i.e., an agent (e.g., a molecule) that inhibits the interaction between ICOS and B7RP-1 or otherwise inhibits signaling through the ICOS-B7RP-1 pathway or that inhibits signaling through the B7RP-1 pathway. Such an agent may be administered, in certain embodiments, to abrogate clinical symptoms, e.g., infiltration of central nervous system (CNS) leukocytes and/or induction of pro-inflammatory cytokines and chemokines in the CNS.

The invention also provides methods of screening potential agents in order to select an agent that is an ICOS-B7RP-1 inhibitor. The present invention also provides methods of identifying agents, e.g., drug screening assays, which agents may be used in therapeutic methods for the treatment of a demyelinating inflammatory disorder of the invention.

According to the methods of the invention, animal models for inflammatory disorders of the invention may be used to screen for agents of the invention that are agonists, antagonists, inhibitors or ligands of ICOS or B7RP-1. Animal models are described herein in Section 4.8.

The present invention provides, in vivo, in situ, and in vitro, methods of identifying an agent (e.g., a drug, compound or pharmaceutical composition) for inhibiting the interaction between ICOS and B7RP-1 or otherwise inhibiting signaling through the ICOS-B7RP-1 pathway, or inhibiting signaling through the B7RP-1 pathway, in a cell or tissue of interest. Such methods can be used alone or in conjunction with each other. A cell or tissue may include, but not be limited to: an excitable cell, e.g., a sensory neuron, motorneuron, or intemeuron; a primary culture of neuronal cells; cell(s) derived from a neuronal cell line; dissociated cell(s); whole cell(s); permeabilized cell(s); a cellular extract or purified enzyme preparation; and a tissue or organ, e.g., brain, brain slice, spinal cord, spinal cord slice, neural tissue or central nervous system tissue. In preferred embodiments, the cell (or tissue) is a neuron (or neural tissue). In a more preferred embodiment, the cell (or tissue) is a neuron (or neural tissue) derived from the central nervous system (CNS).

In one embodiment, the method comprises a method of identifying an ICOS-B7RP-1 inhibitor, comprising (a) contacting an ICOS polypeptide with a B7RP-1 polypeptide expressed on an endothelial cell surface, in the presence of the test compound, under conditions that, in the absence of the test compound, allow the ICOS polypeptide to bind to the B7RP-1 polypeptide and thereby form an ICOS-B7RP-1 complex; and (b) determining whether ICOS-B7RP-1 complex formation is inhibited by the test compound; wherein inhibition of ICOS-B7RP-1 complex formation by the test compound suggests that the test compound is an ICOS-B7RP-1 inhibitor.

In another embodiment, the method further comprises: (c) contacting an ICOS-B7RP-1 complex with a test compound, wherein said B7RP-1 polypeptide is expressed on an endothelial cell surface; and (d) determining whether the ICOS-B7RP-1 complex dissociates following said contacting; wherein dissociation of the ICOS-B7RP-1 complex further suggests that the test compound is an ICOS-B7RP-1 inhibitor.

In another embodiment, the method further comprises: (c) contacting a T-cell capable of being activated by B7RP-1 with B7RP-1, wherein the B7RP-1 expressed on an endothelial cell surface, in the presence of the test compound; and (d) determining whether a lower level of T-cell activation by B7RP-1 occurs in the T-cell after said contacting relative to a control T-cell contacted with B7RP-1 in the absence of the test compound; wherein a lower level of activation further suggests that the test compound is an ICOS-B7RP-1 inhibitor.

In another embodiment, the method further comprises: (c) contacting a T-cell capable of being activated by B7RP-1 with B7RP-1, wherein the B7RP-1 expressed on an endothelial cell surface, in the presence of the test compound; and (d) determining whether a lower level of ICOS activation by B7RP-1 occurs in the T-cell after said contacting relative to a control T-cell contacted with B7RP-1 in the absence of the test compound; wherein a lower level of activation further suggests that the test compound is an ICOS-B7RP-1 inhibitor.

In another embodiment, the invention provides a method of identifying an ICOS-B7RP-1 inhibitor, comprising (a) contacting an ICOS-B7RP-1 complex with a test compound, wherein said B7RP-1 polypeptide is expressed on an endothelial cell surface; and (b) determining whether the ICOS-B7RP-1 complex dissociates following said contacting; wherein dissociation of the ICOS-B7RP-1 complex further suggests that the test compound is an ICOS-B7RP-1 inhibitor.

In another embodiment, the method further comprises (c) contacting a T-cell capable of being activated by B7RP-1 with B7RP-1, wherein the B7RP-1 expressed on an endothelial cell surface, in the presence of the test compound; and (d) determining whether a lower level of T-cell activation by B7RP-1 occurs in the T-cell after said contacting relative to a control T-cell contacted with B7RP-1 in the absence of the test compound; wherein a lower level of activation further suggests that the test compound is an ICOS-B7RP-1 inhibitor.

In another embodiment, the method further comprises (c) contacting a T-cell capable of being activated by B7RP-1 with B7RP-1, wherein the B7RP-1 expressed on an endothelial cell surface, in the presence of the test compound; and (d) determining whether a lower level of ICOS activation by B7RP-1 occurs in the T-cell after said contacting relative to a control T-cell contacted with B7RP-1 in the absence of the test compound; wherein a lower level of activation further suggests that the test compound is an ICOS-B7RP-1 inhibitor.

In another embodiment, the invention provides a method of identifying an ICOS-B7RP-1 inhibitor, comprising (a) contacting a T-cell capable of being activated by B7RP-1 with B7RP-1, wherein the B7RP-1 expressed on an endothelial cell surface, in the presence of a test compound; and (b) determining whether a lower level of ICOS activation by B7RP-1 occurs in the T-cell after said contacting relative to a control T-cell contacted with B7RP-1 in the absence of the test compound; wherein a lower level of activation suggests that the test compound is an ICOS-B7RP-1 inhibitor.

In other embodiments, the method of the invention further comprises, prior to steps (a) disclosed above, identifying a suitable test compound by a method comprising contacting an ICOS polypeptide with a B7RP-1 polypeptide, under conditions that, in the absence of a candidate molecule, allow the ICOS polypeptide to bind to the B7RP-1 polypeptide and thereby form an ICOS-B7RP-1 complex; and determining whether ICOS-B7RP-1 complex formation is inhibited by the candidate molecule; wherein inhibition of ICOS-B7RP-1 complex formation by the candidate molecule suggests that the candidate molecule is suitable test compound.

In other embodiments, the method of the invention further comprises, prior to steps (a) disclosed above, identifying a suitable test compound by a method comprising contacting an ICOS-B7RP-1 complex with a candidate test compound; and determining whether the ICOS-B7RP-1 complex dissociates following said contacting; wherein dissociation of the ICOS-B7RP-1 complex by the candidate molecule suggests that the candidate molecule is suitable as a test compound.

In another embodiment, the invention provides a method of identifying an ICOS-B7RP-1 inhibitor in a cell or tissue of interest that comprises administering the agent to a non-human mammal. The amount (and/or rate) of activity (e.g., expression) of ICOS and/or B7RP-1 is then determined. An agent is identified as an ICOS-B7RP-1 inhibitor when the amount (and/or rate) of activation of T-cells and/or ICOS activity is decreased in the presence of the agent relative to in the absence of the agent. In preferred embodiments, the non-human mammal is a rodent. In a more preferred embodiment, the rodent is a mouse.

In a specific embodiment, the method is performed in vitro. In another specific embodiment, the method is performed in vivo.

In certain embodiments, ICOS activity and/or B7RP-1 activity may include, but not be limited to expression of ICOS and/or B7RP-1, respectively.

In certain embodiments, ICOS polypeptide is expressed on a T cell. In other embodiments, ICOS polypeptide is immobilized on a solid surface. In yet other embodiments, the ICOS polypeptide is present in a cell membrane, which cell membrane is immobilized on the solid surface. In yet other embodiments, the ICOS polypeptide is directly immobilized on the solid surface.

According to the methods of the invention, expression of ICOS and/or its ligand B7RP-1 may be screened for and analyzed using any method commonly known in the art. In certain embodiments, such methods may also be used to assay for activation of T-cells. For example, the methods of McAdam et al. (McAdam, A. J. et al. Mouse inducible costimulatory molecule (ICOS) expression is enhanced by CD28 costimulation and regulates differentiation of CD4(⁺) T cells. J. Immunol. 165, 5035-5040 (2000)) may be used to screen for and analyze ICOS activity, B7RP-1 activity and/or activation of T-cells in the presence and in the absence a potential agent of the invention.

In another embodiment, the methods of Yoshinaga et al. (Yoshinaga, S. K. et al. T-cell co-stimulation through B7RP-1 and ICOS. Nature 402, 827-832 (1999)) are used to screen for and analyze ICOS activity, B7RP-1 activity and/or activation of T-cells in the presence and in the absence a potential agent of the invention.

In another embodiment, the methods of Mages et al. (Mages, H. W. et al Molecular cloning and characterization of murine ICOS and identification of B7h as ICOS ligand. Eur. J. Immunol. 30, 1040-1047 (2000)) are used to screen for and analyze ICOS activity, B7RP-1 activity and/or activation of T-cells in the presence and in the absence a potential agent of the invention.

In another embodiment, the methods of Hutloff et al. (Hutloff, A. et al., ICOS is an inducible T-cell co-stimulator structurally and functionally related to CD28. Nature 397, 263-266 (1999)) and McAdam et al. (2001, ICOS is critical for CD40-mediated antibody class switching. Nature 409, 102-105) are used to screen for and analyze ICOS activity, B7RP-1 activity and/or activation of T-cells in the presence and in the absence a potential agent of the invention. In specific embodiments, these methods may also be used to screen for interleukin 10 (IL-10) expression, CD40 ligand (CD40L) up-regulation and/or TH function for B cell maturation, which may decrease or be inhibited if the ICOS-B7RP-1 pathway is inhibited.

In one embodiment, ICOS activity, B7RP-1 activity and/or activation of T-cells is screened for and analyzed, in the presence and in the absence a potential agent of the invention, using the methods described in Section 5. In one embodiment, ICOS mRNA and/or B7RP-1 mRNA expression in brain specimens from EAE mice (see Section 5) is analyzed.

Standard northern analysis, as commonly practiced in the art, may be used to screen for expression of ICOS and/or B7RP-1.

In another embodiment, ribonuclease protection assay (RPA) analysis of total RNA in the brain may be used to screen for and analyze mRNA expression in the brain, including expression of ICOS and/or B7RP-1. In one embodiment, the RPA methods described in Section 5. Briefly, at various time-points after treatment, experimental subjects, e.g., mice, are killed by CO₂ asphyxiation and the brains and spinal cords are removed. Subsequently, one-half of the brain and a section of thoracic spinal cord is frozen in OCT (Tissue Tek) for immunohistological analysis. The other half of the brain and the remainder of the spinal cord are snap-frozen in liquid nitrogen for RNA isolation using the methods of Chomczynski et al. (Chomczynski, P. & Sacchi, N. Single-step method of RNA isolation by acid guanidinium thiocyanate-phenol-chloroform extraction. Anal. Biochem. 162, 156-159 (1987)).

To prepare B7RP-1 and ICOS probes for northern blot analysis, cloning of B7RP-1 or ICOS cDNA can be accomplished using any method known in the art, e.g., standard RT-PCR methods. In one embodiment, cloning of B7RP-1 and/or ICOS cDNA is accomplished using the methods disclosed in Section 5. Briefly, total RNA is isolated from murine spleens (Chomczynski, P. & Sacchi, N. Single-step method of RNA isolation by acid guanidinium thiocyanate-phenol-chloroform extraction. Anal. Biochem. 162, 156-159 (1987)). The ProStar RT-PCR System (Stratagene, La Jolla, Calif.) is used for B7RP-1 cDNA generation with the primers 5′-GACTGAAGTCGGTGCAATGG-3′ (forward) (SEQ ID NO: 9) and 5′-CTTTCTGCCTGGCTAATGCTAG-3′ (reverse) (SEQ ID NO: 10). The 642-bp B7RP-1 cDNA fragment can be gel-purified and cloned into a Bluescript vector for use as a probe in northern blot analysis. To prepare an ICOS probe for northern blot analysis, ICOS cDNA is prepared using a full-length ICOS plasmid (Incyte Genomics, St. Louis, Mo.). A 556-bp EcoRI-BamHI fragment (EcoRI from the vector), which contains 45 bp 5′-untranslated sequences and a large part of the ICOS coding sequence (the first 170 amino acids of ICOS) is subcloned into a Bluescript vector and used as a probe in northern blot analysis.

Northern analysis of ICOS and/or B7RP-1 expression may be accomplished using standard methods known in the art. In one embodiment, methods disclosed in Section 5 are used for northern analysis. Briefly, total brain RNA (15 μg) is loaded onto each lane of a 1.2% agarose-formaldehyde gel. After electrophoresis, the RNA is blotted overnight onto a Nytran Supercharge membrane (Schleicher and Schuell, Keene, N.H.) with 20×SSC and cross-linked onto the membrane by ultraviolet irradiation using a Stratalinker (Stratagene). Probes to ICOS and B7RP-1 are prepared with the Multiprime Labeling System and [³²P]dCTP (Amersham Pharmacia Biotech, Piscataway, N.J.) and hybridizations are done at 68° C. with ExpressHyb Solution (Clontech Laboratories, Palo Alto, Calif.).

To monitor changes in ICOS and/or B7RP-1 mRNA expression (e.g., from infiltrating T cells), samples (e.g., serial brain samples) may be analyzed by ribonuclease protection assay (RPA) to detect ICOS and/or B7RP-1 mRNA and protein expression by methods commonly known in the art. In one embodiment, the methods for RPA disclosed in Section 5 are used. In a specific embodiment, CD3 expression is also analyzed. CD3 is a T-cell marker and its expression may be screened for using methods well known in the art to assess for the presence and/or numbers of T cells in a given sample.

CNS mRNA may also be quantified by, e.g., RPA, for additional factors of interest, the expression of which may be up-regulated in the disease state of the invention, and inhibited or down-regulated by ICOS-B7RP-1 inhibition, e.g., eotaxin, Ltn, monocyte chemoattractant protein 1 (MCP-1), macrophage-inflammatory protein 1α (MIP-1α), MIP-1β, MIP-2, macrophage migration inhibitory factor (MIF), RANTES, T cell activation 3 (TCA-3), chemokine receptor 1 (CCR1), CCR2, CCR3, CCR5, CXCR1, CXCR2, CXCR4, CXCR5 (V28), IL-1α, IL-1β, IL-2, IL-3, IL-4, IL-5, IL-6, IL-10, IL-12p35, IL-13, IL-15, IL-18, CD3, CD4, CD8, CD45, F4/80 or brain interferon-^{γ} (IFN-^{γ}). In preferred embodiments, the expression of IL-1α, IL-1^{γ}, IL-6, IFN-^{γ}, MCP1 and/or CCR1 is screened for in the presence and in the absence of a test compound.

Accordingly, in one embodiment, the invention provides a method of identifying an ICOS-B7RP-1 inhibitor, comprising contacting a T-cell capable of being activated by B7RP-1 with B7RP-1, wherein the B7RP-1 expressed on an endothelial cell surface, in the presence of a test compound; and determining whether a lower level of T-cell activation by B7RP-1 occurs in the T-cell after said contacting relative to a control T-cell contacted with B7RP-1 in the absence of the test compound; wherein a lower level of activation suggests that the test compound is an ICOS-B7RP-1 inhibitor. In a specific embodiment, the method is performed in vitro. In another specific embodiment, the method is performed in vivo. In another specific embodiment, T-cell activation is indicated by an increase in the expression of MCP-1, CCR 1, interleukin-1α, interleukin-1β, interleukin-6, interleukin-10, or interferon-γ. In another specific embodiment, T cell activation is evidenced by the ability of the T cell to traverse an in vitro model of the blood brain barrier.

In another specific embodiment, described in Section 5, expression of one or more CNS mRNAs is quantified by RPA, according to manufacturer's instructions (Riboquant, PharMingen). Briefly, 15 μg aliquots of RNA are hybridized with [α-P³²]UTP-labeled riboprobes complementary to the factor of interest and the housekeeping gene G3PDH. After hybridization, samples are digested with the RNAse A/T1, separated on a polyacrylamide gel and analyzed by autoradiography. To measure the relative abundance of mRNAs, gels are analyzed with a Phosphorimager (Molecular Devices, Sunnyvale, Calif.); sample-to-sample variation in RNA loading is controlled by expressing the data as a fraction of the G3PDH signal: target:G3PDH ratio=target cpm/G3PDH cpm.

To monitor changes in ICOS and/or B7RP-1 mRNA expression in T cells, e.g., infiltrating T-cells in the brain, serial samples (e.g., brain samples) may be analyzed by immunohistochemical (IHC) analysis of specimens from subjects to detect ICOS and/or B7RP-1 mRNA expression (and in certain embodiments, CD3 mRNA expression and/or expression of another protein or factor of interest) by methods commonly known in the art. In one embodiment, the immunohistochemical methods disclosed by Gonzalo et al. (2001, Nature Immunol. 2:597-604) are used.

In a specific embodiment, an ICOS-reporter gene construct, e.g, an ICOS-IRES-GFP, construct may be constructed, and transgenic animals or transformed cell lines expressing the construct may be generated using methods commonly known in the art. Expression of such a construct in the transgenic animal or cell line may be used to monitor ICOS expression and/or T cell activation. In another specific embodiment, a B7RP-1-reporter gene construct, e.g., an B7RP-1-IRES-GFP, construct may be constructed, and transgenic animals or transformed cell lines expressing the construct generated. Expression of such a construct in the transgenic animal or cell line may be used to monitor B7RP-1 expression and/or T cell activation.

In another embodiment, the methods for immunohistochemical analysis disclosed in Section 5 are used. Briefly, tissue sections, e.g., brain and/or spinal cord sections, are fixed in cold acetone and washed in PBS with 1% gelatin. Tissues are then blocked with PBS with 10% fetal bovine serum (FBS, Hyclone, Logan, Utah) and 10% goat serum (Sigma) for 30 min. The blocking solution is then shaken off and replaced with 10 μg/ml of hamster anti-mouse CD3 or rat anti-mouse ICOS (mAb 12A8) overnight at 4° C. The next day, sections are washed in PBS with 1% gelatin and incubated with a labeled (e.g., biotinylated) goat anti-hamster antibody (Vector Laboratories, Burlingame, Calif.) or a labeled (e.g., biotinylated) mouse anti-rat-lgG2b antibody (BD Pharmingen) for 30 min at room temperature. After another wash, the label is developed, e.g., avidin-biotin complexes (ABC Elite; Vector Laboratories, Burlingame, Calif.) are added to slides and incubated for 30 min at room temperature. After a final wash, slides are developed with diaminobenzidine (DAB), counterstained with Meyer's haematoxylin, dehydrated and cover-slips are added. The histological specimens are then examined under a microscope (e.g., a light or fluorescence microscope) and the labeling pattern is visualized and analyzed.

To monitor changes in ICOS and/or B7RP-1 mRNA expression (e.g., from infiltrating T cells), in certain embodiments, tissue samples (e.g., serial brain samples) may also be analyzed by flow cytometric analyses to detect ICOS and/or B7RP-1 mRNA (and, in certain embodiments, CD3 mRNA and/or expression of another protein or factor of interest, as described herein) by methods commonly known in the art. Standard methods for serial flow cytometric analysis of tissues such as blood and brain may be used to screen for, e.g., a decrease (or increase) in ICOS⁺ T cells or blood or brain leukocytes.

In one embodiment, a serial flow cytometric analysis of cells, e.g., brain and/or blood leukocytes, may be carried out using the methods disclosed in Sections 5 and 6. Briefly, brain or blood leukocytes may be isolated as disclosed in Sedgwick, J. D. et al. (Isolation and direct characterization of resident microglial cells from the normal and inflamed central nervous system. Proc. Natl Acad. Sci. USA 88, 7438-7442 (1991)). Then, after blockade in PBS with 10% FBS and 10% goat serum, cell (e.g., blood and brain leukocyte) samples are incubated with 10 μg/ml of rat anti-mouse ICOS (mAb 12A8) for 30 min. After a wash step, cells are incubated with biotin-conjugated mouse anti-rat IgG2b (PharMingen). After another wash, cells are blocked with rat serum and then incubated with hamster FITC-anti-mouse CD3 for 30 min. Samples are lysed (FACslyse, BD PharMingen) and analyzed on a FACSTAR flow cytometer (BD PharMingen).

In certain embodiments, the assay system used to identify ICOS-B7RP-1 inhibitors involves preparing a reaction mixture containing at least the ICOS- binding portion of B7RP-1 and the B7RP-1-binding portion of ICOS under conditions (referred to in this section as the B7RP-1 protein and the ICOS protein, respectively) and for a time sufficient to allow the two to interact and bind, thus forming a complex. In order to test a compound for inhibitory activity, the reaction mixture is prepared in the presence and in the absence of a potential agent of the invention. The test compound can be initially included in the reaction mixture, or can be added at a time subsequent to the addition of the ICOS and B7RP-1 proteins. Control reaction mixtures are incubated without the test compound or with a placebo. The formation of any complexes between the ICOS and B7RP-1 is then detected. The formation of a complex in the control reaction, but not in the reaction mixture containing the test compound, indicates that the compound interferes with the ICOS-B7RP-1 interaction.

Assays for potential ICOS and B7RP-1 inhibitors can be conducted in a heterogeneous or homogeneous format. Heterogeneous assays involve anchoring either the ICOS and B7RP-1 protein onto a solid phase and detecting complexes anchored on the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction between ICOS and B7RP-1, e.g., by competition, can be identified by conducting the reaction in the presence of the test compound; i.e., by adding the test compound to the reaction mixture prior to or simultaneously with the ICOS and B7RP-1 proteins. Alternatively, test compounds that disrupt preformed complexes, e.g. compounds with higher binding constants that displace one of the components from the complex, can be tested by adding the test compound to the reaction mixture after complexes have been formed. The various formats are described briefly below.

In a heterogeneous assay system, either the ICOS and B7RP-1 protein, is anchored onto a solid surface, while the non-anchored species is labeled, either directly or indirectly. In practice, microtiter plates are conveniently utilized. The anchored species can be immobilized by non-covalent or covalent attachments. Non-covalent attachment can be accomplished simply by coating the solid surface with a solution of the ICOS and B7RP-1 protein and drying. Alternatively, an immobilized antibody specific for the species to be anchored can be used to anchor the species to the solid surface. The surfaces can be prepared in advance and stored.

In order to conduct the assay, the partner of the immobilized species is exposed to the coated surface with or without the test compound. After the reaction is complete, unreacted components are removed (e.g., by washing) and any complexes formed will remain immobilized on the solid surface. The detection of complexes anchored on the solid surface can be accomplished in a number of ways. Where the non-immobilized species is pre-labeled, the detection of label immobilized on the surface indicates that complexes were formed. Where the non-immobilized species is not pre-labeled, an indirect label can be used to detect complexes anchored on the surface; e.g., using a labeled antibody specific for the initially non-immobilized species (the antibody, in turn, can be directly labeled or indirectly labeled with a labeled anti-Ig antibody). Depending upon the order of addition of reaction components, test compounds which inhibit complex formation or which disrupt preformed complexes can be detected.

In a specific embodiment, ICOS protein-expressing cell membranes or purified ICOS proteins are immobilized on a solid surface, and the binding of a partner, e.g., a labelled B7RP-1 fusion protein (labelled with, e.g., a fluorochrome label or a radioactive label such as ³⁵S or ¹²⁵I) is assayed. In another embodiment, B7RP-1 protein-expressing cell membranes or purified B7RP-1 proteins are immobilized on a solid surface, and the binding of a partner, e.g., a labelled ICOS fusion protein, is assayed. Such an embodiment may be easily adapted by the skilled practitioner to any robotic or high throughput screening format commonly known in the art.

Alternatively, the reaction can be conducted in a liquid phase in the presence and in the absence of the test compound, the reaction products separated from unreacted components, and complexes detected; e.g., using an immobilized antibody specific for one of the binding components to anchor any complexes formed in solution, and a labeled antibody specific for the other partner to detect anchored complexes. Again, depending upon the order of addition of reactants to the liquid phase, test compounds which inhibit complex or which disrupt preformed complexes can be identified.

In an alternate embodiment of the invention, a homogeneous assay can be used. In this approach, a preformed complex of the ICOS and B7RP-1 proteins is prepared in which either the ICOS and B7RP-1 protein is labeled, but the signal generated by the label is quenched due to complex formation (see, e.g., U.S. Pat. No. 4,109,496 by Rubenstein which utilizes this approach for immunoassays). The addition of a test substance that competes with and displaces one of the species from the preformed complex will result in the generation of a signal above background. In this way, test substances which disrupt ICOS-B7RP-1 interaction can be identified.

In a particular embodiment, the target gene product can be prepared for immobilization using recombinant DNA techniques known to those of skill in the art. For example, the ICOS or B7RP-1 protein can be fused to a glutathione-S-transferase (GST) gene using a fusion vector, such as pGEX-5×-1, in such a manner that its binding activity is maintained in the resulting fusion protein. The binding partner (i.e., the B7RP-1 or ICOS protein, respectively) can be purified and used to raise a monoclonal antibody, using methods routinely practiced in the art and described above. This antibody can be labeled with the radioactive isotope ¹²⁵I, for example, by methods routinely practiced in the art. In a heterogeneous assay, e.g., the ICOS or B7RP-1 fusion protein can be anchored to glutathione-agarose beads. The B7RP-1 or ICOS protein, respectively, can then be added in the presence and in the absence of the test compound in a manner that allows interaction and binding to occur. At the end of the reaction period, unbound material can be washed away, and the labeled monoclonal antibody can be added to the system and allowed to bind to the complexed components. The interaction between ICOS and B7RP-1 can be detected by measuring the amount of radioactivity that remains associated with the glutathione-agarose beads. A successful inhibition of the interaction by the test compound will result in a decrease in measured radioactivity.

Alternatively, the GST-ICOS or GST-B7RP-1 fusion protein and its binding partner (i.e., IB7RP-1 or ICOS protein, respectively) can be mixed together in liquid in the absence of the solid glutathione-agarose beads. The test compound can be added either during or after the species are allowed to interact. This mixture can then be added to the glutathione-agarose beads and unbound material is washed away. Again, the extent of inhibition of the ICOS-B7RP-1 interaction can be detected by adding the labeled antibody and measuring the radioactivity associated with the beads.

In one embodiment of the foregoing methods, the test compound is a peptide fragment that corresponds to the extracellular portion of ICOS or B7RP-1, thereby allowing the identification of small ICOS-B7RP-1 inhibitor peptides that can be produced synthetically instead of recombinantly for use in the present methods and compositions.

In another embodiment, the invention provides methods of screening for agents that modulate activity of ICOS and/or B7RP-1 wherein experimental animals are divided into at least three groups, preferably ten per group, that either received no treatment, intraperitoneal treatment with e.g., 100 μg of a candidate agent during the antigen priming phase (e.g., days 1-10 in a mouse EAE model) or intraperitoneal treatment with 100 μg of the candidate during the efferent response phase (e.g., on days 9-20 in a mouse EAE model). Data collected may be, e.g, the mean response ±s.e.m. of ten replicates.

In one embodiment of the invention, the ability of an agent of the invention to inhibit interaction of ICOS and B7RP-1 is screened for in an animal model. For example, in an animal model (e.g., an EAE mouse), the subject may be treated with a potential agent of the invention either during antigen priming (e.g., days 1-10 in an EAE mouse) or during an efferent immune response (e.g., days 9-20 in an EAE mouse) (see Section 5). The onset of disease in the treated animal may be compared with those in an untreated control group (e.g., on day 14 in an EAE mouse) (see Section 5).

In another embodiment, the method of the invention comprises administering to a model animal with experimental allergic encephalomyelitis the test compound during the efferent stage of said experimental allergic encephalomyelitis; and determining whether ICOS positive T cells traverse the blood brain barrier of said model animal at a reduced rate relative to a model animal with experimental allergic encephalomyelitis to whom the test compound is not administered; wherein a reduction of rate of traversal of the blood brain barrier further suggests that the test compound is an ICOS-B7RP-1 inhibitor.

In another embodiment, the method of the invention comprises administering to a model animal with experimental allergic encephalomyelitis the test compound during the efferent stage of said experimental allergic encephalomyelitis; and determining whether brain inflammation is reduced in said model animal relative to a model animal with experimental allergic encephalomyelitis to whom the test compound is not administered; wherein a reduction of brain inflammation further suggests that the test compound is an ICOS-B7RP-1 inhibitor.

In another embodiment, the method of the invention comprises administering to a model animal with experimental allergic encephalomyelitis the test compound during the efferent stage of said experimental allergic encephalomyelitis; and determining whether physical symptoms of experimental allergic encephalomyelitis are reduced in the model animal relative to a model animal with experimental allergic encephalomyelitis to whom the test compound is not administered; wherein a reduction of physical symptoms of experimental allergic encephalomyelitis further suggests that the test compound is an ICOS-B7RP-1 inhibitor.

In another embodiment, the method of the invention provides a method of identifying an ICOS-B7RP-1 inhibitor, comprising (a) administering to a model animal with experimental allergic encephalomyelitis a test compound during the efferent stage of said experimental allergic encephalomyelitis; and (b) determining whether physical symptoms of experimental allergic encephalomyelitis are reduced in the model animal relative to a model animal with experimental allergic encephalomyelitis to whom the test compound is not administered; wherein a reduction of physical symptoms of experimental allergic encephalomyelitis suggests that the test compound is an ICOS-B7RP-1 inhibitor. In a specific embodiment, the model animal is a mouse.

In another embodiment, the methods of Kopf et al. (Kopf, M. et al. Inducible costimulator protein (ICOS) controls T helper cell subset polarization after virus and parasite infection. J. Exp. Med. 192, 53-61 (2000)) may be used to screen for T-cell activation via the ICOS- B7RP-1 pathway by an agent of the invention in an animal model in which an immune response to a virus, bacterium or parasite (e.g., an intestinal parasite) has been experimentally induced.

Severity scores for symptoms of a disorder of the invention in treated subjects and in control groups may be measured and recorded as described in Section 5. For example, in one embodiment, the severity scores for symptoms of a disorder of the invention may be, e.g., 0, normal; 1, ataxia; 2, loss of muscle tone (e.g., tail tone in rodents); 3, posterior paresis and loss of righting response; 4, tetraparesis; 5, moribund.

In addition, standard microscopic examination of tissue samples (e.g., brain samples) collected from treated and untreated subjects may be examined during various time points after administration of the potential agent, e.g., during the antigen priming phase. Infiltrating cells, such as T cells, B cells, monocytes, macrophages or neutrophils may be screened for.

In certain embodiments, northern analysis may be used, as described herein, to screen for expression of brain ICOS and/or B7RP-1 mRNA after administration of a potential agent of the invention.

Expression of ICOS and/or B7RP-1 in specimens (e.g., brain or CNS specimens) from naïve or wildtype animals may be analyzed and compared with expression in experimental animals, for example, at various time points after treatment with an agent of the invention. In one embodiment, infiltration of the meninges and neuropil with CD3⁺-, ICOS-, or B7RP-1-expressing cells is screened for using the methods of Section 5. In a particular embodiment, serial sections of the brain are screened.

In another embodiment, changes in various chemokines, chemokine receptors, cytokines, or leukocyte markers are measured after administration of a potential agent of the invention as an additional test of ICOS-B7RP-1 inhibition. Various chemokines, chemokine receptors, cytokines, and leukocyte markers have been implicated in the pathogenesis of at least one of animal model for a disorder of the invention, EAE (Rottman, J. B. et al. Leukocyte recruitment during onset of experimental allergic encephalomyelitis is CCR1 dependent. Eur. J. Immunol. 30, 2372-2377 (2000); Karpus, W. J. et al. An important role for the chemokine macrophage inflammatory protein-1 in the pathogenesis of the T cell-mediated autoimmune disease, experimental autoimmune encephalomyelitis. J. Immunol. 155, 5003-5010 (1995); Constantinescu, C. S. et al. Modulation of susceptibility and resistance to an autoimmune model of multiple sclerosis in prototypically susceptible and resistant strains by neutralization of interleukin-12 and interleukin-4, respectively. Clin. Immunol. 98, 23-30 (2001); Izikson, L. et al. Resistance to experimental autoimmune encephalomyelitis in mice lacking the CC chemokine receptor (CCR)2. J. Exp. Med. 192, 1075-1080 (2000); Glabinski, A. R. et al. Synchronous synthesis of α- and β-chemokines by cells of diverse lineage in the central nervous system of mice with relapses of chronic experimental autoimmune encephalomyelitis. Am. J. Pathol. 150, 617-630 (1997); Okuda, Y. et al. Enhancement of Th2 response in IL-6-deficient mice immunized with myelin oligodendrocyte glycoprotein. J. Neuroimmunol. 105, 120-123 (2000)). To determine whether administration of an agent inhibits the ICOS-B7RP-1 pathway, and, e.g., alters expression of mediators such as chemokines, the methods disclosed in Section 5 may be used. Briefly, brain mRNA samples are collected from the various experimental (treated or untreated) groups at the time point of expected onset of disease symptoms (e.g., the efferent phase in EAE mice) by RPA. If there is no difference detected in the mRNA expression of various chemokines or chemokine receptors of naïve animals and animals that have been treated with an agent of the invention at the time of expected onset of the disease or disorder (or its symptoms), then the potential agent is scored as an ICOS-B7RP-1 inhibitor. Various chemokines, chemokine receptors, cytokines, or leukocyte markers, the mRNAs of which can be screened for, include, but are not limited to, eotaxin, Ltn, monocyte chemoattractant protein 1 (MCP-1), macrophage-inflammatory protein 1α (MIP-1α), MIP-1β, MIP-2, macrophage migration inhibitory factor (MIF), RANTES, T cell activation 3 (TCA-3), chemokine receptor 1 (CCR1), CCR2, CCR3, CCR5, CXCR1, CXCR2, CXCR4, CXCR5 (V28), IL-1α, IL-1β, IL-2, IL-3, IL-4, IL-5, IL-6, IL-10 IL-12p35, IL-13, IL-15, IL-18, CD3, CD4, CD8, CD45, F4/80 or brain interferon-^{γ} (IFN-^{γ}).

In another embodiment, the ability of a potential agent of the invention to inhibit the ICOS-B7RP-1 pathway and thereby inhibit IFN-^{γ} expression is screened for. In embodiments of this type, inhibition of the ICOS-B7RP-1 pathway may result in lowered expression of various chemokines, cytokines, etc., e.g., IFN-^{γ}, IL-4 and IL-10, as discussed above. As disclosed in Section 5, enzyme-linked immunosorbent assay (ELISA) analysis of supernatants from cultured splenocytes may be used to determine whether an experimental animal treated with a potential agent of the invention produces more or less of various chemokines, cytokines, etc. than control animals. Lymphocyte proliferation in model animals treated with an agent of the invention during antigen priming or during the efferent phase may be compared with that in untreated model animals

IFN-^{γ} production and expression can be measured using any method commonly known in the art. For example, as disclosed in Section 5, splenocyte IFN-⁶⁵ production and proliferation may be measured by comparing splenocytes from untreated control, naïve or untreated disease model animals to splenocytes from animals treated with a potential agent of the invention. Animals treated with a potential agent of the invention during the antigen priming and/or efferent phase of a disease or disorder may be compared. For example, in certain embodiments, model animals treated with a potential agent of the invention during the efferent phase of the disease or disorder show less splenocyte proliferation and IFN-^{γ} expression than other treatment groups, whereas those treated with a potential agent of the invention during the antigen priming phase show greater splenocyte proliferation and IFN-^{γ} expression than other treatment groups.

Splenocytes may be cultured under conditions commonly known in the art. In one embodiment, disclosed in Section 5, splenocytes are isolated from the various treatment groups, cultured, and counted.

In certain embodiments, the ability of a potential agent of the invention to inhibit the ICOS-B7RP-1 pathway is screened for by examining B cell maturation. In preferred embodiments, inhibition of the ICOS-B7RP-1 pathway will result in decreased immunoglobulin G1 (IgG1) and IgG2a production, and can be examined using methods well known in the art (see, e.g., Coyle, A. J. et al. The CD28-related molecule ICOS is required for effective T cell-dependent immune responses. Immunity 13, 95-105 (2000)).

In one embodiment, the methods disclosed in Section 5 are used to determine whether inhibition of the ICOS-B7RP-1 pathway alters the humoral response in an animal model. For example, as disclosed in Section 5, total plasma IgG1 and plasma disease-specific (e.g., PLP-specific) IgG1 may be measured in plasma samples collected from various treatment groups at a particular time point during the onset or exhibition of the disease or disorder, e.g. during the efferent phase of an autoimmune disease. In one embodiment, according to the methods disclosed in Section 5, among the various treated and untreated groups, animals treated with an ICOS-B7RP-1 inhibitor, during antigen priming, should have a higher concentration of plasma IgG1 and show significant decrease in disease-induced IgG1 concentration compared with untreated disease model animals. By contrast, animals treated during the efferent disease component will show similar concentrations of plasma disease-specific IgG1 as untreated disease model controls. In one embodiment, the analysis is carried out using the ELISA methods disclosed in Section 5 are used.

Agents of the invention may also be screened for based on their ability to prevent opening of the blood-brain barrier (BBB). As demonstrated in Section 5.3, inhibition of the ICOS-B7RP-1 pathway inhibits opening of the BBB. As disclosed in Section 5.3, activation of T cells through the ICOS-B7RP-1 pathway is necessary for opening of the BBB in an autoimmune disorder. An agent that inhibits activation of the pathway and of T-cells may be screened for by treating animals in vivo with a dose (or doses) of a candidate agent prior to T cell entry into the brain. The brain is then later screened for evidence, e.g., by Western analysis, of opening of the BBB. For example, as disclosed in Section 5.3, animal subjects for a model of an ICOS-B7RP-1 pathway disorder (e.g., EAE) may be given an injection of rabbit serum on days 7, 8 and 9 of the antigen priming phase, to determine if the BBB was permeable to macromolecules. Animals are then euthanized on day 10 of the antigen priming phase and brain homogenates may be studied, e.g., by Western analysis, to detect rabbit Ig leakage into the neuropil. An agent of the invention is scored as anti-ICOS, anti-B7RP-1 or as an inhibitor of the ICOS-B7RP-1 pathway if it inhibits opening of the BBB.

Accordingly, in another embodiment, the invention provides a method of identifying an ICOS-B7RP-1 inhibitor, comprising (a) administering to a model animal with experimental allergic encephalomyelitis a test compound during the efferent stage of said experimental allergic encephalomyelitis; and (b) determining whether ICOS positive T cells traverse the blood brain barrier of said model animal at a reduced rate relative to a model animal with experimental allergic encephalomyelitis to whom the test compound is not administered; wherein a reduction of rate of traversal of the blood brain barrier suggests that the test compound is an ICOS-B7RP-1 inhibitor. In a specific embodiment, the model animal is a mouse.

Agents of the invention may also be screened for based on their effect on the activation of T-cells and/or the ability of T-cells to infiltrate the blood-brain barrier (BBB) in vitro. In such embodiments, BBB endothelial cells may be co-cultured with T-cells in vitro using standard culture methods. In one embodiment, the methods disclosed in Section 5.3 are used. Briefly, Multisorb 96 well plates (NUNC, lctn) are coated with various concentrations of antibody, e.g. anti-CD3ε antibody, in PBS overnight at 4° C. Subsequently, the plates are washed 4 times with PBS/1% gelatin and various combinations of sub-confluent endothelial cells (b.END.3) and/or T cells (2×10⁵/well) are placed in culture along with antibody (anti-ICOS or control at 10 μg/ml) and/or TNF-α (100 ng/ml; R&D Systems). Cells are cultured, in the presence or in the absence of a test agent, at 37° C., 5% CO₂ for 48 hours. 0.5 μCi of ³H-thymidine is then added to each well for an additional 16 hours. Cells may be subsequently harvested onto unifilter microplates and counted (Topcount, Packard Instrument, Downers Grove, Ill.).

In another embodiment, agents of the invention may also be screened for in an in vitro assay using the methods disclosed in Prat et al. (2002, Migration of multiple sclerosis lymphocytes through brain endothelium, Arch. Neurol. 59(3):391-7). Briefly, lymphocytes, e.g., lymphocytes derived from the peripheral blood of an animal model or a patient with MS, are assayed in an artificial model of the blood-brain barrier in the presence or the absence of a test compound. A solid surface such as a chamber (e.g., a Boyden chamber) is coated with a monolayer of human brain microvascular endothelial cells and the rates of migration of lymphocytes obtained from experimental subjects or patients is measured. ICOS activity, B7RP-1 activity, and/or activation of T-cells may be assessed, e.g., using ribonuclease protection assays or enzyme-linked immunosorbent assays (ELISAs).

As disclosed above, in one embodiment, an endothelial cell line, b.END.3 may be used in an in vitro assay of the invention. Cell lines that can be used in assays of the invention other than b.END.3 include, but are not limited to the following:

| Cells | Origin | Described in |
|---|---|---|
| EA.hy926 | human endothelial cell | Leszczynski et al., 2002, Differentiation 70(2-3): 120-9 |
| | line | |
| MBEC4 | mouse brain endothelial | Hosoya et al., J Pharmacol. Exp. Ther. 302(1):225-31 |
| | cell line | |
| RBE4 | rat brain microvessel | Calhau et al., 2002, Naunyn Schmiedebergs Arch |
| | endothelial cell line | Pharmacol 365(5):349-56 |
| GP8.3 | rat brain-derived | Koedel et al., 2002, J Cereb Blood Flow Metab 22(1):39- |
| | endothelial cell line | 49 |
| BBMECs | bovine brain | Cox et al., 2001, J Pharm Sci 90(10):1540-52 |
| | microvessel endothelial | |
| | cells | |
| TM-BBB | conditionally | Takanaga et al., 2001, J Cereb Blood Flow Metab |
| | immortalized mouse | 21(10): 1232-9 |
| | brain capillary | |
| | endothelial cell line | |
| rBCEC4 | rat brain capillary | Blasig et al., 2001, Microvasc Res 62(2):114-27 |
| | endothelial cells | |
| | immortalized with | |
| | polyoma virus large T | |
| | antigen | |
| T24/83 | rat cerebral endothelial | Tan et al., 2001, Neuroreport 12(7): 1329-34 |
| | cells | |
| Eahy929 | rat cerebral endothelial | Tan et al., 2001, Neuroreport 12(7): 1329-34 |
| | cells | |
| b.End5 | rat cerebral endothelial | Tan et al., 2001, Neuroreport 12(7): 1329-34 |
| | cells | |
| RBEC1 | immortalized cell line | Tamai et al., 2000, J Drug Target 8(6):383-93 |
| | from rat brain capillary | |
| | endothelial cells | |
| HBMEC | human brain | Zysk et al., 2001, Infect Immun 69(2):845-52 |
| | microvascular | |
| | endothelial cell line | |
| SV-HCEC | new human | Duvar et al., 2000, J Neurochem 75(5):1970-6 |
| | cerebromicrovascular | |
| | endothelial cell | |
| | immortalized with | |
| | SV40 large T antigen | |

### 4.6 ANIMAL MODELS

According to the methods of the invention, ICOS-B7RP-1 inhibition during an autoimmune response may be used to abrogate clinical symptoms, central nervous system (CNS) leukocyte infiltration and induction of pro-inflammatory cytokines and chemokines in the CNS. According to the methods of the invention, animal models for immune or autoimmune disorders may be used to screen for ICOS-B7RP-1 inhibitors. In preferred embodiments, the immune or autoimmune disorder is a demyelinating inflammatory disorder.

In one embodiment of the invention, a mouse model of experimental allergic encephalomyelitis (EAE) may be used (Perrin, P. J. et al. Blockade of CD28 during in vitro activation of encephalitogenic T cells or after disease onset ameliorates experimental autoimmune encephalomyelitis. J. Immunol. 163, 1704-1710 (1999)). EAE is a prototypic T_{H}1-mediated demyelinating disease that is used as a model for human multiple sclerosis (Wekerle, H. Imrnunopathogenesis of multiple sclerosis. Acta. Neurol. Napoli 13, 197-204 (1991)). EAE may be induced by methods well known in the art. In one embodiment, EAE may be induced by injecting an animal subject subcutaneously in a single sit (e.g., at the tail base in a rodent) with 100 μg of proteolipid protein (PLP) 139-151 emulsified in complete Freund's adjuvant (Sigma, St. Louis, Mo.) supplemented with 4 mg/ml Mycobacterium tuberculosis antigen (Difco, Detroit Mich.) in a total volume of 100 μl.

In another embodiment, EAE may be induced by using the methods of Perrin et al. ((Perrin, P. J. et al. Blockade of CD28 during in vitro activation of encephalitogenic T cells or after disease onset ameliorates experimental autoimmune encephalomyelitis. J. Immunol. 163, 1704-1710 (1999)).

In another embodiment that uses an EAE animal model, an agent is screened for its ability to inhibit the ICOS-B7RP-1 pathway during the efferent immune response to proteolipid protein (PLP). The EAE animal model is screened for abrogation or amelioration of clinical symptoms, central nervous system (CNS) leukocyte infiltration and induction of pro-inflammatory cytokines and chemokines in the CNS according to the methods described in Section 5. In another embodiment, an agent is screened for its ability to inhibit the ICOS-B7RP-1 pathway during antigen priming. The EAE animal model is screened for polarization of a T_{H}1 response to PLP, enhanced or reduced expression of pro-inflammatory cytokines and chemokines in the CNS, and exacerbation or amelioration of brain leukocyte infiltration and clinical symptoms, as described in Section 5. In another embodiment, the EAE animal model is screened for CNS demyelination according to methods well known in the art (see, e.g., Wekerle, Immunopathogenesis of multiple sclerosis. Acta. Neurol. Napoli 13, 197-204 (1991); Perrin et al. Blockade of CD28 during in vitro activation of encephalitogenic T cells or after disease onset ameliorates experimental autoimmune encephalomyelitis. J. Immunol. 163, 1704-1710 (1999)).

In another embodiment of the invention, a mouse model of collagen-induced arthritis (CIA) is used (see, e.g., Tada, Y. et al. CD28-deficient mice are highly resistant to collagen-induced arthritis. J. Immunol. 162, 203-208 (1999)).

In another embodiment of the invention, a mouse model of asthma is used (see, e.g., Mathur, M. et al. CD28 interactions with either CD80 or CD86 are sufficient to induce allergic airway inflammation in mice. Am. J. Respir. Cell. Mol. Biol. 21, 498-509 (1999))

In another embodiment, a CD28-deficient mouse model is used (Kopf, M. et al. Inducible costimulator protein (ICOS) controls T helper cell subset polarization after virus and parasite infection. J. Exp. Med. 192, 53-61 (2000)). The methods of Kopf et al., may be used to screen for increased or decreased costimulation of T cells via the ICOS- B7RP-1 pathway by an agent of the invention in an experimentally induced immune response to a virus, bacterium or parasite (e.g., an intestinal parasite).

In another embodiment of the invention, ICOS-deficient mice are used (Dong, C. et al. ICOS co-stimulatory receptor is essential for T-cell activation and function. Nature 409, 97-101 (2001); Tafuri, A. et al. ICOS is essential for effective T-helper-cell responses. Nature 409, 105-109 (2001)). Such mice may be used in assays for additional confirmation that an agent of the invention exerts its effects via the ICOS-B7RP-1 pathway. In certain embodiments, a candidate ICOS-B7RP-1 inhibitor is tested for its effects on the activation of T cells in ICOS-deficient mice using a screening method of the invention described hereinabove. If the candidate ICOS-B7RP-1 inhibitor has no effect on T cell activation in ICOS-deficient mice, this would provide additional evidence that the candidate is likely to exert its effects via inhibition of the ICOS-B7RP-1 pathway.

### 4.7 PHARMACEUTICAL PREPARATIONS AND METHODS OF ADMINISTRATION

The immunosuppressive agents and ICOS-B7RP-1 inhibitors that are useful in the present methods and compositions, such as those described herein, can be administered to a patient in amounts effective to treat or prevent a demyelinating inflammatory disorder of the central nervous system.

### 4.7.1 EFFECTIVE DOSE

Toxicity and therapeutic efficacy of ICOS-B7RP-1 inhibitory compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic indices are preferred. While compounds that exhibit toxic side effects can be used, care should be taken to design a delivery system that targets such compounds to the target cells in order to minimize potential damage to unaffected cells and, thereby, reduce side effects.

The data obtained from the cell culture assays and animal studies can be used in formulating a range of dosage for use in humans. The dosage of ICOS-B7RP-1 inhibitor lies preferably within a range of circulating concentrations that include the ED₅₀ with little or no toxicity. The dosage can vary within this range depending upon the dosage form employed and the route of administration utilized. For any ICOS-B7RP-1 inhibitor used in the method of the invention, the therapeutically effective dose can be estimated initially from cell culture assays. A dose can be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma can be measured by any technique known in the art, for example, by high performance liquid chromatography.

For human clinical trials of ICOS-B7RP-1 inhibitors, several methods are available for determining a useful therapeutic outcome. Measurement of TIWGd+activity and change in T2W lesion burden are useful as indicators of outcomes in patients who recently have experienced frequent relapses (Goodkin, 1996, Mult Scler 1(6):393-9J). The “summary measure” statistic AUC incorporates both transient and progressive disability into an overall estimate of the dysfunction that was experienced by a patient during a period of time (Liu et al., 1998, Neurol Neurosurg Psychiatry 64(6):726-9). Commonly used methods of statistical analysis which are relevant to the evaluation of the results of randomized controlled clinical trials in multiple sclerosis are described by Petkau, 1998, SeminNeurol 18(3):351-75.

### 4.7.2 FORMULATIONS AND USE

The invention relates to pharmaceutical compositions and methods of use thereof for preventing or treating a central nervous system demyelinating inflammatory disorder. Such pharmaceutical compositions can be formulated in a conventional manner using one or more physiologically acceptable carriers or excipients.

Thus, the compounds and their physiologically acceptable salts and solvents can be formulated for systemic administration or local administration at the site of the blood-brain barrier. Further, the compounds can be formulated for administration by inhalation or insufflation (either through the mouth or the nose) or oral, buccal, parenteral or rectal administration.

For oral administration, the pharmaceutical compositions can take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets can be coated by methods well known in the art. Liquid preparations for oral administration can take the form of, for example, solutions, syrups or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate.

Preparations for oral administration can be suitably formulated to give controlled release of the active compound.

For buccal administration the compositions can take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver metered amount. Capsules and cartridges of, e.g., gelatin for use in an inhaler or insufflator can be formulated containing a powder mix of the compound and a suitable powder base such as lactose or starch.

The compounds can be formulated for parenteral administration (i.e., intravenous or intramuscular) by injection, via, for example, bolus injection or continuous infusion. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents. Alternatively, the active ingredient can be in powder form for constitution with a suitable vehicle, e.g., sterile pyrogen-free water, before use.

The compounds can also be formulated in rectal compositions such as suppositories or retention enemas, e.g., containing conventional suppository bases such as cocoa butter or other glycerides.

In addition to the formulations described previously, the compounds can also be formulated as a depot preparation. Such long acting formulations can be administered by implantation (for example subcutaneously or intramuscularly) or by intramuscular injection. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

### 4.8 KITS

The present invention provides kits for practicing the methods of the present invention. A kit of the invention comprises in one or more containers an ICOS-B7RP-1 inhibitor, such as those described in Section 4.2, supra, and, optionally, a second therapeutic agent, for example an immunosuppressive agent, such as those described in Section 4.3, supra.

The kit of the invention may optionally comprise additional components useful for performing the methods of the invention. By way of example, the kit may comprise pharmaceutical carriers useful for formulating the ICOS-B7RP-1 inhibitor. Where the ICOS-B7RP-1 inhibitor is administered in the form of cell therapy or gene therapy, suitable cells or gene therapy vectors may also be included. In addition, the kits of the invention may further provide an instructional material which describes performance of the methods of the invention, or a notice in the form prescribed by a governmental agency regulating the manufacture, use or sale of pharmaceuticals or biological products, which notice reflects approval by the agency of manufacture, use or sale for human administration.

In one embodiment, the present invention provides kits for practicing the screening methods of the present invention. A screening kit of the invention may comprise, in certain embodiments, in one or more containers, sample(s) of cell(s )or tissue(s) of interest, e.g., endothelial cells and/or lymphocytes, primary cultures of cells, cells derived from a neuronal or endothelial cell line; dissociated cell(s); whole cell(s); permeabilized cell(s); a cellular extract or a purified enzyme preparation.

By way of example, the kit can provide a sample of endothelial cells and/or lymphocytes, and optionally, a chamber or solid surface that has been (or can be) coated with a monolayer of the endothelial cells so that rates of migration of lymphocytes that are provided with the kit (or obtained from experimental subjects or patients) can be measured as described above. The kit may optionally provide instructions and/or materials for performing an expression assay, e.g., a ribonuclease protection assay or an enzyme-linked immunosorbent assay (ELISA) to assess for ICOS activity, B7RP-1 activity, and/or activation of T-cells, as described above.

Also by way of example, the kit may provide a solid surface (e.g., a culture plate) upon which ICOS protein-expressing cell membranes or purified ICOS proteins (or B7RP-1 protein-expressing cell membranes or purified B7RP-1 proteins) are immobilized, and with which the binding of a partner, e.g., a labelled B7RP-1 fusion protein (or labelled ICOS fusion protein) is assayed.

The screening kit of the invention may optionally comprise additional components useful for performing the methods of the invention. By way of example, the components of the kit may also optionally include, but are not limited to: labelled B7RP-1 fusion protein (labelled with, e.g, a fluorochrome label or a radioactive label such as ³⁵S or ¹²⁵I); anti-ICOS antibody; anti-B7RP-1 antibody; and an antisense compound(s) of the invention. In addition, the screening kits of the invention may further provide instructional material as described herein.

The following experimental examples are offered by way of illustration and not by way of limitation.

### 5. EXAMPLE

### THE ROLES OF THE ICOS-B7RP-1 PATHWAY IN THE IMMUNOPATHOGENESIS OF EAE

The studies described herein show that blockade of the ICOS-B7RP-1 pathway during the efferent immune response to PLP results in the prevention of clinical disease associated with decreased splenocyte proliferation and IFN-γ expression in response to PLP; abrogation of brain chemokine, chemokine receptor and cytokine mRNA expression; and inhibition of CNS leukocyte infiltration. Thus, these data suggest that the ICOS-B7RP-1 pathway plays a central role in the immunopathogenesis of EAE. Accordingly, drugs designed to block this pathway provide effective treatment for selected patients with multiple sclerosis.

The data presented herein further provide evidence that encephalitogenic T cells are limited to the ICOS+ population of brain-infiltrating lymphocytes and that these cells may be activated at the level of the BBB by interaction with activated brain endothelial cells which express B7RP-1. Blockade of B7RP-1 perhaps inhibits activation of these encephalitogenic ICOS+ T cells by endothelium and subsequent opening of the BBB. Collectively, the data herein suggest that the ICOS/B7RP-1 pathway can be targeted for treatment of inflammatory diseases of the nervous system such as multiple sclerosis.

### 5.1 MATERIALS AND METHODS

Animals: Female SJL/J mice, 6-8 weeks of age, were from the Jackson Laboratory (Bar Harbor, Me.).

EAE induction:Animals were injected subcutaneously in a single site at the tail base with 100 μg of proteolipid protein (PLP) 139-151 emulsified in complete Freunds adjuvant (Sigma, St. Louis, Mo.) supplemented with 4 mg/ml Mycobacterium tuberculosis antigen (Difco, Detroit Mich.) in a total volume of 100 μl.

Experimental design: Immunized animals were divided into three groups (n=ten per group) that either received no treatment, intraperitoneal treatment with 100 μg of mAb 12A8 on days 1-10 (antigen priming) or intraperitoneal treatment with 100 μg of mAb 12A8 on days 9-20 (efferent response). For data on blockade efficacy, two EAE experiments were run: identical results were obtained. Data are mean ±s.e.m. of ten replicates.

MAb 12A8: This mAb is a rat-anti-mouse ICOS, isotype IgG2b, that blocks binding of the ligand B7RP-1 to murine ICOS transfectant cells. The antibody has a half-life of approximately 14 h in vivo and, based upon flow cytometric analysis and immunohistology, does not deplete ICOS⁺ T cells from peripheral blood or tissues. In vivo treatment of mice with this antibody elicits a strong neutralizing anti-rat response, which begins by day 12 of treatment (Özkaynak et al., 2001, Nature Immunol. 2:591-596). Antibody 8F3 is a rat-anti-mouse B7RP-1 antibody, isotype IgG2a (Millennium Pharmaceuticals). The control antibody YK9 is a rat monoclonal, isotype IgG2a.

Disease scoring system: Mice were weighed and scored daily. Scoring was based upon the following scale: 0, normal; 1, ataxia; 2, loss of tail tone; 3, posterior paresis and loss of righting response; 4, tetraparesis; 5, moribund.

Tissue collection and total RNA preparation: At various time-points after immunization, mice were killed by CO₂ asphyxiation and the brains and spinal cords were removed. Subsequently, one-half of the brain and a section of thoracic spinal cord were frozen in OCT for immunohistological analysis. The other half and remainder of the spinal cord were snap-frozen in liquid nitrogen for RNA isolation (Chomczynski and Sacchi, 1987, Anal. Biochem. 162:156-159).

Cloning of B7RP-1 cDNA by RT-PCR: Total RNA was isolated from murine spleens. The ProStar RT-PCR System (Stratagene, La Jolla, Calif.) was used for B7RP-1 cDNA generation with the primers 5′-GACTGAAGTCGGTGCAATGG-3′ (forward) (SEQ ID NO: 9) and 5′-CTTTCTGCCTGGCTAATGCTAG-3′ (reverse) (SEQ ID NO: 10). The 642-bp B7RP-1 cDNA fragment was gel-purified and cloned into a Bluescript vector for use as a probe in northern blot analysis. ICOS cDNA: the full-length ICOS plasmid was from Incyte Genomics (St. Louis, Mo.). A 556-bp EcoRI-BamHI fragment (EcoRI from the vector), which contained 45 bp 5′-untranslated sequences and a large part of the ICOS coding sequence (the first 170 amino acids of ICOS) was subdloned into a Bluescript vector and used as a probe in northern blot analysis.

Northern analysis of ICOS and B7RP-1 expression: Total brain RNA (15 μg) was loaded onto each lane of a 1.2% agarose-formaldehyde gel. After electrophoresis, the RNA was blotted overnight onto a Nytran Supercharge membrane (Schleicher and Schuell, Keene, N.H.) with 20×SSC and cross-linked onto the membrane by ultraviolet irradiation using a Stratalinker (Stratagene). Probes to ICOS and B7RP-1 were prepared with the Multiprime Labeling System and [³²P]dCTP (Amersham Pharmacia Biotech, Piscataway, N.J.) and hybridizations were done at 68° C. with ExpressHyb Solution (Clontech Laboratories, Palo Alto, Calif.). For reuse, membranes were deprobed in 0.5% SDS at 95-100° C. and exposed to film to assure complete removal of previous probes.

Preparation of splenic ICOS+ and ICOS− T cells: Spleens were harvested from either naïve or PLP-immunized SJL/J mice 10 days PI. Spleens were aseptically removed, mechanically dissociated, and red blood cells removed by hypotonic lysis. The remaining leukocytes were washed twice and suspended at 5×10⁶ cells/ml in media (RPMI 1640 (Gibco) supplemented with 0.1 mM nonessential amino acids, 1 mM sodium pyruvate, 2 mM L-glutamine, 100 U/ml of penicillin, 100 U/ml of streptomycin, 10% heat-inactivated fetal bovine serum (Biowhittaker, Walkersville, Md.) and 5×10⁻⁵ M 2-mercaptoethanol (Sigma Chemical Co., St. Louis, Md.)). Total splenocytes were subsequently cultured for 2 hours at a concentration of 5×10⁶ cells/ml in media at 37° C., 5% CO₂ to allow APCs to adhere to the plastic. Subsequently, non-adherent cells were adjusted to a concentration of 10⁸ cells/ml, incubated for 15 minutes at 4° C. with anti-mouse CD19 coated magnetic beads and depleted on a magnetic column as per manufacturers instructions (Miltenyi Biotech, Auburn, Calif.). Flow-through cells were adjusted to a concentration of 5×10⁶ cells/ml in media and incubated with 10 μg/ml anti-ICOS antibody 12A8 at 4° C. for 30 minutes. Following a wash step, cells were incubated with rat-anti-mouse IgG2b-specific magnetic beads (Miltenyi Biotech) at 4° C. for 15 minutes and separated on a magnetic column (Miltenyi Biotech). The flow-through ICOS− cells were collected, the adherent ICOS+ cells were eluted from the column, and both populations were adjusted to a concentration of 10⁷ cells/ml in media. Because ICOS is expression was highly variable among activated cells, it was difficult to separate the cells into strict ICOS+ and ICOS− populations. Thus, lymphocytes with high levels of ICOS expression separated with the ICOS+ population whereas lymphocytes that were devoid of ICOS expression or had such low levels that they did not adhere to the magnetic column separated with the ICOS− population.

Preparation of splenocytes for use as APCs: Total splenocytes were prepared as above and incubated with 25 μg/ml mitomycin C (Sigma, St Louis) at room temperature for 30 minutes. Subsequently, splenocytes were washed 4 times and resuspended in media at a concentration of 2×10⁶/ml.

Co-culture of ICOS+ and ICOS− splenocytes and splenic APCs: 24-well plates were seeded with 2×10⁶ naïve SJL/J splenocytes in media. Subsequently, 2×10⁶ ICOS+ or ICOS− splenic T cells from naïve or immunized mice were added to each well in the presence or absence of PLP (100 μg/ml) and/or anti-ICOS or isotype control antibodies (10 μg/ml). Cells were cultured for 48 hours at 37° C., 5% CO₂.

IFN-γ ELISPOT analysis of ICOS+ and ICOS− T cells: Lymphocytes were harvested from the aforementioned splenic co-cultures, washed, and 10⁵ cells were added to triplicate wells in ELISPOT plates and incubated overnight at 37° C., 5% CO₂ The plates were subsequently developed to detect IFN-γ according to manufacturers instructions (R&D Systems, Minneapolis Minn.) and read by Zellnet Consulting (New York, N.Y.) on a Zeiss automated ELISPOT reader to determine the number of spot-forming cells per well.

Isolation of brain leukocytes: 14 days PI, SJL/J mice were euthanized by CO₂ asphyxiation and brain leukocytes were isolated as previously described (Sedgwick, 1991, Proc Natl Acad Sci USA 88:7438-42). Briefly, mice were perfused by injecting 3 mls 5 mM EDTA in PBS into the left ventricle and allowing the blood to escape from an incision in the right atrium. The brain and spinal cord were then dissected and placed in PBS+4% FCS on ice. The frontal lobes of the brain were removed and the remaining brain tissue and spinal cord were placed in separate wells containing 150 μl of collagenase D (Boehringer Mannheim, cat#1088-858). The tissue was subsequently minced and incubated at 37° C. 5%, CO₂ for 45 minutes. After incubation, the brain cell suspension was diluted to 5 mls in PBS and layered over a Percoll gradient (Percoll, Amersham Pharmacia Biotech AB; 1.131 g/mL stock; 3.11 mL Percoll+5.89 mL 4% FCS). The gradients were centrifuged at 1700 RPM for 15 minutes at room temperature. The supernatant was subsequently removed, and the cell pellet washed 3 times with 9 mL of PBS/4% FCS.

Flow cytometric separation of brain T cells into ICOS+ and ICOS− populations All procedures were performed at 4° C. After isolation, brain leukocytes were blocked in PBS/10% goat serum for 15 minutes. Following a wash, cells were resuspended to a concentration of 10⁷ cells/ml, antibody 12A8 was added to a concentration of 20 μg/ml and cells were incubated for 30 minutes to label ICOS+ T cells. After washing, cells were subsequently incubated with 10 μg/ml biotinylated mouse-anti-rat IgG2b (BD Pharmingen) for 30 minutes, washed and then incubated with streptavidin PE at 1:400 (Southern Biotech) for an additional 30 minutes. After another wash, cell pellets were blocked with rat serum. Finally, hamster-anti-mouse CD3-FITC was added and cells were incubated for 15 minutes. Following a final wash step, cells were sorted into CD3+ICOS+ and CD3+ICOS− populations on a FACSTAR flow cytometer (BD Pharmingen).

Brain T cell culture conditions: Brain CD3+ICOS+ and CD3+ICOS− T cells (2×10⁵/well) were incubated in media with mitomycin C—treated splenocytes from naïve SJL/J mice (4×10⁵/well) in the presence or absence of PLP (100 μg/ml). Cells were cultured for 72 hours at 37° C., 5% CO₂ and supernatants were subsequently harvested for further analysis.

ELISA for IFN-γ: Supernatants form the brain T cell cultures were analyzed for IFN-γ expression according to manufacturers instructions (R&D Systems, Minneapolis Minn.). Data is presented as the average of three replicates ±SEM.

Enrichment of Mac1+ spleen and brain APCs: Total splenocytes or brain leukocytes prepared as previously described were adjusted to a concentration of 10⁸/ml and incubated with anti-Mac-1 coated magnetic beads at 4° C. for 60 minutes. Cells were subsequently added to a magnetic column, which was washed and adherent cells were eluted according to the manufacturers instructions (Miltenyi Biotech).

Generation of recently activated T cells: Spleens were removed from SJL/J mice on day 10 PI, total splenocytes prepared as previously described and placed in culture at 2×10^{6/}ml with PLP (100 μg/ml) for 72 hours. Splenocytes were subsequently washed, layered over a ficoll gradient (Lymphoprep, lctn) and centrifuged to remove dead cells. The remaining viable cells were placed in culture with IL-2 (10 IU/ml) in media for 3 to 5 days. Cells were subsequently sorted into ICOS+ and ICOS− T cells as described above and adjusted to 2×10⁶/ml in media for subsequent study.

APC/recently activated T cell co-culture studies: To study the ability of splenic or brain Mac1+ APCs to present antigen to recently activated T cells, spleen or brain Mac-1+e nriched APCs were added to 96 well plates in triplicate (10⁵/well) with ICOS+ or ICOS− cells (2×10⁵/well), in the presence or absence of PLP. In some studies, anti-ICOS antibody (10 μg/ml) was also added. Cells were cultured at 37° C., 5% CO₂ for 72 hours and 0.5 μCi of ³H-thymidine was added to each well for an additional 16 hours. Cells were subsequently harvested onto unifilter microplates and counted (Topcount, Packard Instrument, Downers Grove, Ill.).

Immunohistology: Brain and spinal cord sections were fixed in cold acetone and washed in PBS with 1% gelatin. Tissues were then blocked with PBS with 10% fetal bovine serum (FBS, Hyclone, Logan, Utah) and 10% goat serum (Sigma) for 30 min. The blocking solution was shaken off and replaced with 10 μg/ml of hamster anti-mouse CD3 or rat anti-mouse ICOS (mAb 12A8) overnight at 4° C. The next day, sections were washed in PBS with 1% gelatin and incubated with biotinylated goat anti-hamster (Vector Laboratories, Burlingame, Calif.) or biotinylated mouse anti-rat-lgG2b (BD Pharmingen) for 30 min at room temperature. After another wash, avidin-biotin complexes (ABC Elite; Vector Laboratories, Burlingame, Calif.) were added to slides and incubated for 30 min at room temperature. After a final wash, slides were developed with diaminobenzidine (DAB), counterstained with Meyer's hematoxylin, dehydrated and cover-slips were added.

Flow cytometric analysis of blood and brain leukocytes: Brain leukocytes were isolated as described (Sedgwick et al., 1991, Proc. Natl Acad. Sci. USA 88:7438-7442). After blockade in PBS with 10% FBS and 10% goat serum, blood and brain leukocyte samples were incubated with 10 μg/ml of rat anti-mouse ICOS (mAb 12A8) for 30 min. After a wash step, cells were incubated with biotin-conjugated mouse anti-rat IgG2b (PharMingen). After another wash, cells were blocked with rat serum and then incubated with hamster FITC-anti-mouse CD3 for 30 min. Samples were lysed (FACslyse, BD PharMingen) and analyzed on a Becton-Dickinson FACscan instrument (BD PharMingen).

RPA: CNS mRNA for eotaxin, Ltn, MCP-1, MIP-1α, MIP-1^{β}, MIP-2, macrophage migration inhibitory factor (MIF), RANTES, T cell activation 3 (TCA-3), CCR1, CCR2, CCR3, CCR5, CXCR1, CXCR2, CXCR4, CXCR5 (V28), IL-1α, IL-1β, IL-2, IL-3, IL-4, IL-5, IL-6, IL-10, IL-12p35, IL-13, IL-15, IL-18, CD3, CD4, CD8, CD45 and F4/80 were quantified by RPA, according to manufacturer's instructions (Riboquant, PharMingen). Briefly, 15 μg aliquots of RNA were hybridized with [α-P³²]UTP-labeled riboprobes complimentary to the aforementioned targets and the housekeeping gene G3PDH. After hybridization, samples were digested with the RNAse A/T1, separated on a polyacrylamide gel and analyzed by autoradiography. To measure the relative abundance of mRNAs, gels were analyzed with a Phosphorimager (Molecular Devices, Sunnyvale, Calif.); sample-to-sample variation in RNA loading was controlled by expressing the data as a fraction of the G3PDH signal: target:G3PDH ratio=target cpm/G3PDH cpm.

Splenocyte culture conditions: Splenocytes were isolated from the various treatment groups on day 12 after immunization and 5 106 cells/ml were cultured in RPMI 1640 (Gibco-BRL, Gaithersburg, Md.) supplemented with 0.1 mM nonessential amino acids, 1 mM sodium pyruvate, 2 mM L-glutamine, 100 U/ml of penicillin, 100 U/ml of streptomycin, 10% heat-inactivated FBS (Biowhittaker, Walkersville, Md.) and 5 10⁻⁵ M 2-mercaptoethanol (Sigma). To study cytokine expression, splenocytes were cultured with various dilutions of PLP (100, 10, 1 or 0 μ/ml) at 37° C., 5% CO₂ and supernatants collected at 72 h for analysis by ELISA. For cell proliferation studies, splenocytes were similarly cultured with dilutions of PLP for 72 h and pulsed with 0.5 μCi of [³H]thymidine for an additional 16 h. Cells were subsequently collected onto unifilter microplates and counted (Topcount, Packard Instrument, Downers Grove, Ill.).

ELISA for cvtokines: Quantitative ELISAs for mouse IL-4, IL-10 and IFN-^{γ} were performed using cytokine-specific kits per the manufacturer's recommendations (R&D Systems, Minneapolis, Minn.). Data are mean ±s.e.m. of three separate experiments.

ELISA for determination of plasma PLP-specific IgG1 concentration: Polycarbonate 96 well plates (Nunc, Roskilde, Denmark) were coated overnight at 4° C. with standard curve lanes (2.5 μg/ml goat-anti-mouse) and test lanes (2 μg/ml PLP in carbonate buffer, pH 9.3). Plates were washed and blocked with 2% bovine serum albumin in PBS, and dilutions of purified IgG1 (MOPC-21, Sigma) and mouse plasma were added to control and test lanes, respectively. After a 2-h incubation at 37° C., plates were washed and peroxidase-conjugated goat-anti-mouse was added with 5% rat serum and incubated for 1 h at 37° C. After final washes, plates were developed with ortho-phenylenediamine in citrate buffer pH 5.0 and the reaction was stopped with 12.5% sulfuric acid. Plates were read at 490 nm on a Spectramax Plus plate reader and analyzed with the Softmax Pro software (Molecular Devices). Data are mean ±s.e.m. of three separate experiments.

Northern analysis of endothelial cells: Northern analysis of B7RP-1 expression. 15 μg of total endothelial RNA was loaded onto each lane of a 1.2% agarose-formaldehyde gel. After electrophoresis, the RNA was blotted overnight onto a Nytran Supercharge membrane (Schleicher and Schuell, Keene, N.H.) with 20×SSC and cross-linked onto the membrane by UV irradiation using a Stratalinker (Stratagene). Probes to B7RP.1 were prepared using the Multiprime Labeling System and 32P-dCTP (Amersham Pharmacia Biotech, Piscataway, N.J.) and hybridizations were performed at 68° C. using ExpressHyb Solution (Clontech Laboratories, Palo Alto, Calif.). For re-use, membranes were deprobed in 0.5% SDS at 95-100° C. and exposed to film to assure complete removal of previous probes.

Endothelium/T cell coculture experiments: Multisorb 96 well plates (NUNC, lctn) were coated with various concentrations of anti-mouse CD3ε in PBS overnight at 4° C. Subsequently, the plates were washed 4 times with PBS/1% gelatin and various combinations of sub-confluent endothelial cells (B end 3) and/or recently PLP-activated T cells (2±10⁵/well) were placed in culture along with antibody (anti-ICOS or control at 10 μg/ml) and/or TNF-α (100 ng/ml; R&D Systems). Cells were cultured at 37° C., 5% CO₂ for 48 hours and 0.5 μCi of ³H-thymidine was added to each well for an additional 16 hours. Cells were subsequently harvested onto unifilter microplates and counted (Topcount, Packard Instrument, Downers Grove, Ill.).

Statistical analysis: Statistical significance between groups was shown with the Student's t-test.

Genbank accession numbers: Full-length-ICOS plasmid has the accession number ai006009.

### 5.2 RESULTS: THE COSTIMULATORY MOLECULE ICOS PLAYS AN IMPORTANT ROLE IN THE IMMUNOPATHOGENESIS OF EAE

Early up-regulation of brain ICOS and B7RP-1: The kinetics of ICOS and B7RP-1 mRNA expression were examined in brain specimens from SJL mice immunized with proteolipid protein PLP(1 39-151), referred to hereafter as PLP. Northern analysis of brain mRNA samples collected at various times after immunization with PLP showed that ICOS mRNA expression was undetectable in noninflamed brain (days 0 and 7). However, ICOS mRNA was markedly up-regulated before onset of disease symptoms (day 10) and expression persisted through day 20. In contrast, mRNA of B7RP-1, the ligand for ICOS, was constitutively expressed in low amounts, but up-regulated in parallel with ICOS by day 10, returning to baseline expression by day 20. Thus, both ICOS and B7RP-1 mRNA were up-regulated markedly before disease onset, which suggested that this costimulatory pathway was important in the pathogenesis of this disease.

Brain ICOS expression limited to infiltrating T cells: To date, it has been reported that ICOS expression is limited to activated T cells. To prove that the source of increased ICOS mRNA expression was infiltrating T cells, serial brain samples were analyzed by ribonuclease protection assay (RPA), immunohistochemical (IHC) and flow cytometric analyses to detect CD3 and ICOS mRNA and protein expression. In naïve brain specimens, RPA showed minimal CD3 mRNA expression; but in immunized animals, brain CD3 mRNA expression increased on day 10 and was maximal on day 12. CD3-immunoreactive cells were first detected in the brain on day 10 and infiltration of the meninges and neuropil with CD3+ cells was maximal by day 12. In serial sections, IHC showed that ICOS protein expression was limited to a subset (10-30%) of the infiltrating mononuclear cells. Serial flow cytometric analysis of blood and brain showed that ICOS+ T cells preferentially accumulated in the brain during disease progression and ICOS expression was limited to a small subpopulation (up to 12%) of brain CD3+ T cells. This was consistent with immunohistology data. Thus, ICOS protein and mRNA expression correlate with brain T cell infiltration, and ICOS is expressed exclusively by a subset of the infiltrating T cells.

ICOS blockade can abrogate or enhance disease: If ICOS plays an important costimulatory role in the pathogenesis of EAE, ICOS blockade with a specific monoclonal antibody (mAb) should abrogate disease. Thus, mice were treated with the murine ICOS-blocking mAb 12A8 either during antigen priming (days 1-10) or during the efferent immune response to PLP (days 9-20). Compared with the untreated control group on day 14 (incidence=10/10, severity score=3.0±0.6), ICOS blockade with mAb 12A8 during the efferent immune response abrogated disease (0/10) through day 19. After day 19, a subset of animals (5/10) developed disease (severity scores varied from 1 to 3), coincident with the appearance of mAb 12A8-neutralizing antibodies. In contrast, ICOS blockade during antigen priming resulted in more severe clinical symptoms by day 14 (incidence=10/10, severity score=5.0±0.0) compared with the untreated control group. Microscopic examination of brain samples collected from mice on day 14 showed that, compared with naïve mice or immunized, untreated mice, ICOS blockade during antigen priming resulted in a more robust leukocyte infiltrate. The infiltrate was characterized by an increase in T cells, B cells, monocytes or macrophages and a large increase in neutrophils. Also within this group, northern analysis showed, by day 14, a marked increase in the expression of brain ICOS and B7RP-1 mRNA, which correlated with the appearance of mAb 12A8-neutralizing antibodies. In contrast, animals treated during the efferent stage of the response to PLP did not have brain leukocyte infiltration, and ICOS and B7RP-1 mRNA were either undetectable or barely detectable, respectively. Thus, there was a profound difference in disease course and brain leukocyte infiltration that was dependent upon whether ICOS blockade occurred during antigen priming or during the effector phase of the immune response.

Changes in brain cvtokines during ICOS blockade: Various chemokines, chemokine receptors and cytokines have been implicated in the pathogenesis of EAE (Rottman et al., 2000, Eur. J. Immunol. 30:2372-2377; Karpus et al., 1995, J. Immunol. 155: 5003-5010; Constantinescu et al., 2001, Clin. Immunol. 98:23-30; Izikson et al., 2000, J. Exp. Med. 192:1075-1080; Glabinski et al., 2000, Am. J. Pathol. 150:617-630; Okuda et al., 2000, J. Neuroimmunol. 105:120-123). To determine how ICOS blockade altered expression of these important mediators, brain mRNA samples collected from the various groups on day 14 were measured RPA. There was no difference in the mRNA expression of naïve animals and animals that had been treated with anti-ICOS during the efferent immune response. Thus, ICOS blockade during the efferent immune response abrogated disease at clinical, cellular and molecular levels. In contrast, the two groups of animals that developed clinical disease (immunized, no treatment and immunized, treated during antigen priming) had a twofold or greater increase in certain mRNAs. Of the mRNAs that were up-regulated, a subset-chemokine receptor 1 (CCR1), regulated upon activation, normal T cell-expressed and secreted (RANTES), macrophage-inflammatory protein 2 (MIP-2) and monocyte chemoattractant protein 1 (MCP-1), IL-1α, IL-1β, IL-6 and IL-12p35-was higher in the animals treated with anti-ICOS during antigen priming. In addition, brain interferon-γ (IFN-γ) mRNA expression could be detected only in animals treated during antigen priming (minimal expression at the low limits of detection, data not shown). Thus, one or more of the above chemokines, chemokine receptors or cytokines could be responsible for the increased disease severity associated with ICOS blockade during antigen priming.

Afferent ICOS blockade and IFN-γ expression: Immunization of SJL mice with PLP results in the generation of T cells of both TH1 and TH2 phenotypes, which require different amino acid residues on PLP for activation (Das et al., 1997, J. Exp. Med. 186:867-876). Ultimately the TH1cells become dominant and are responsible for causing disease. To determine whether ICOS blockade during antigen priming resulted in a further enhancement of the TH1 cellular response and inhibition of TH2 cell expansion, and whether, in turn, this extreme TH1 polarization was responsible for the enhanced disease symptoms, the expression of IFN-γ, IL-4 and IL-10 was examined following ICOS blockade during antigen priming. Enzyme-linked immunosorbent assay (ELISA) analysis of supernatants from cultured splenocytes collected on day 12 after immunization showed that mice treated with anti-ICOS during antigen priming produced more IFN-γ (38257±15268 pg/ml) than immunized untreated mice (5437±2301 pg/ml, P<0.05). Animals treated with anti-ICOS during antigen priming also had more robust lymphocyte proliferation to PLP than immunized untreated animals. Thus, ICOS blockade during antigen priming enhanced antigen-specific T cell proliferation and IFN-γ expression.

In contrast to the increased T cell proliferation and IFN-γ expression resulting from blocking of ICOS during the T cell priming phase of EAE, splenocytes from mice treated with anti-ICOS during the efferent phase of the disease produced less IFN-γ (1244±581 pg/ml) than the immunized untreated group (P<0.05) and decreased lymphocyte proliferation, as compared with other immunized animals. Thus, ICOS blockade during the efferent immune response appeared to reduce antigen-specific lymphocyte proliferation and IFN-γ expression. In addition, splenocytes from mice treated with anti-ICOS during antigen priming produced low amounts of IL-4 and IL-10, which were not vastly different from other groups, and expression did not increase upon exposure to PLP.

Inhibition of PLP-specific IgG1 production: In addition to providing costimulation for T cells, the ICOS-B7RP-1 costimulatory pathway participates in B cell maturation and blockade results in decreased immunoglobulin GI (IgG1) and IgG2a production (Coyle et al., 2000, Immunity 13:95-105). To determine whether ICOS blockade had altered the humoral response in our models, total plasma IgG1 and PLP-specific IgG1 were measured in plasma samples collected from the various treatment groups 14 days after immunization using ELISA. Among the groups, animals treated with anti-ICOS during antigen priming had the highest concentration of plasma IgG1. These same animals showed a significant decrease in PLP-specific IgG1 concentration (1.4±0.9 μg/ml; P<0.05) compared with immunized untreated animals (13.1±5.1 μg/ml). In contrast, animals treated during the efferent disease component showed similar concentrations of plasma PLP-specific IgG1 as the untreated controls (7.3±1.7 μg/ml, P>0.05). Therefore, the increased severity of clinical disease associated with ICOS blockade during antigen priming cannot be explained by an enhanced humoral response to PLP. Because TH2 cells are known to provide help for B cell production of IgG122, these data provide further evidence that ICOS blockade during antigen priming polarizes the immune response to a TH1 phenotype.

### 5.3 RESULTS: ENCEPHALITOGENIC T CELLS EXPRESS ICOS AND INTERACT WITH B7RP-1+ ENDOTHELIAL CELLS TO MEDIATE EAE

PLP-specific splenic T cells express ICOS. The results described in Section 5.2, supra, demonstrate that ICOS+ T cells infiltrate the brains of PLP-immunized mice on day 10 PI prior to onset of clinical symptoms of EAE. Those experiments also demonstrate that blockade of the ICOS-B7RP-1 interaction with a specific monoclonal antibody during the efferent immune response (days 9 through 20 PI) abrogated disease onset. Based upon these data, it was proposed that ICOS+ T cells might be the PLP-specific, encephalitogenic lymphocytes responsible for initiating disease. If this notion was correct, PLP-specific T cells isolated from secondary lymphoid organs of immunized mice should uniquely be confined to the ICOS+ but not the ICOS− subpopulation. To test this hypothesis magnetic beads were to sort splenic T cells from naïve and PLP-immunized SJL/J mice (day 10 PI) into CD3+ICOS+ and CD3+ICOS− subsets. Since the ligand for ICOS (B7RP-1, B7h, LICOS) is expressed on APCs such as B cells and macrophage (Yoshinaga, 1999, Nature 402:827-32), mitomycin-C treated splenocytes from naïve SJL/J mice were used as APCs. After incubating CD3+ICOS+ and CD3+ICOS− lymphocytes with APCs for 48 hours in the presence or absence of PLP, the cells were then transferred to ELISPOT plates overnight to detect IFN-γ expression. Lymphocytes that were able to respond to PLP and produce IFN-γ were limited to the ICOS+ subpopulation from immunized mice.

PLP-specific, brain-infiltrating T cells express ICOS. Following priming in peripheral lymphoid organs draining the site of immunization, PLP-specific T cells subsequently travel to the brain and mediate disease. Given that PLP-specific T cells in the spleen were limited to the ICOS+ subpopulation, it was proposed that encephalitogenic T cells isolated from the brain of animals with disease should also be limited to the ICOS+ subset. To test this hypothesis, brains from SJL/J mice with EAE (day 14 PI) were isolated and brain T cells were sorted by flow cytometry into CD3+ICOS+ and CD3+ICOS− populations. The T cells were subsequently cultured with mitomycin C—treated splenocytes from naïve SJL/J mice to serve as APCs in the presence or absence of PLP. In these experiments, the CD3+ICOS+, but not the CD3+ICOS− T cells, were activated and produced interferon gamma in response to PLP. These data demonstrate that in animals with EAE, brain PLP-specific T cells are contained within the ICOS+ population and suggest that these cells are the encephalitogenic cells that initiate disease. In contrast, brain CD3+ICOS− T cells do not respond to PLP, and suggest that this subpopulation may represent the T cells that are non-specifically recruited to the brain during disease onset.

Brain APCs express B7RP-1. B7RP-1 mRNA is constitutively expressed in the brain, expression levels increasing in proportion to the severity of brain inflammation (see Section 5, supra). Brain Mac1+CD45^{hi} infiltrating macrophages and Mac1+CD45^{int} microglia express B7-1 and B7-2, present antigen and play an important role in the immunopathogenesis of EAE (Juedes, 2001, J. Immunol. 166:5168-75). It was therefore determined whether these cells also expressed B7RP-1. Brains from SJL/J mice with EAE (day 14 PI) were isolated and subjected to enrichment for Mac1+ cells using magnetic beads. Flow cytometric analysis demonstrated that Mac1+CD45+ cells expressed B7RP-1 and that incubation of the Mac-1+CD45+ enriched cells with LPS for 1 hour increased the level of B7RP-1 expression.

Brain APCs present antigen to ICOS+ T cells. Mac-1+ enriched brain and spleen APCs were prepared and incubated with recently PLP-activated lymphocytes in the presence or absence of specific antigen. APCs from both sources present antigen to PLP-specific, ICOS+ T cells, resulting in enhanced lymphocyte proliferation. Interestingly, antigen presentation could not be inhibited with blocking anti-ICOS antibodies. Thus, although APCs that express B7RP-1 can activate ICOS+ PLP-specific T cells, there are alternative costimulatory pathways that can compensate for blockade of the ICOS/B7RP-1 pathway. However, this observation contrasts with the initial in vivo findings that blockade of the ICOS/B7RP-1 pathway during the efferent phase of the disease inhibits the onset of EAE. In the in vivo study, however, treatment with anti-ICOS began on day 9 PI, one day prior to detectable infiltration of the brain by ICOS+ T cells. Thus, the critical costimulation of PLP-specific ICOS+ T cells through ICOS/B7RP-1 may occur proximal to their entry into the brain and interaction with brain APCs. Hence, B7RP-1 expression along the blood-brain barrier (BBB) was examined.

B7RP-1 is expressed by activated brain endothelium. To determine whether brain endothelial cells could provide a costimulatory signal through ICOS, immunohistochemical analysis of normal and inflamed (EAE day 14 PI) mouse brains was performed. There was patchy expression of B7RP-1 on the endothelium of inflamed, but not normal brain. Also, flow cytometric analysis of an endothelial cell line revealed B7RP-1 expression. To further prove this point, serial Northern analysis of a murine endothelial cell line (B end 3), either resting or following stimulation with TNF-α, was performed. There is minimal constitutive expression of B7RP-1 in resting endothelial cells, but the mRNA is dramatically upregulated upon stimulation with TNF-α. These data suggest that brain endothelial cells potentially could provide a costimulatory signal to PLP-specific ICOS+ T cells at the level of the BBB prior to entry of these encephalitogenic cells into the brain.

Brain endothelial cells provide a costimulatory signal through ICOS/B7RP-1 to activate ICOS+ encephalitogenic T cells. Because no endothelial cell line that was MHC class II-matched for the SJL/J mouse could be identified, test had to be tested indirectly by demonstrating whether endothelial cells could provide costimulation of ICOS+ PLP-specific T cells in trans. Recently activated PLP-specific T cells were incubated with resting or TNF-α stimulated endothelial cells that expressed B7RP-1, in the presence or absence of various concentrations of anti-CD3ε to provide a stimulus through the TCR. B7RP-1+ endothelial cells provided a costimulatory signal to recently activate PLP-specific T cells and this signal could be inhibited by anti-ICOS. Thus, brain endothelial cells have the potential to activate encephalitogenic T cells, either directly or in trans, and it is possible that this activation is necessary for initial opening of the BBB.

Blockade of the ICOS/B7RP-1 pathway inhibits opening of the BBB. Assuming that activation of PLP-specific encephalitogenic T cells through the ICOS/B7RP-1 pathway is necessary for opening of the BBB in EAE, the opening should be inhibited by treating animals in vivo with a single dose of anti-ICOS prior to T cell entry into the brain. To test this hypothesis, EAE was induced in SJL/J mice and the mice treated with either anti-ICOS or control rat Ig on day 9 PI. Animals were also given an injection of rabbit serum on days 7, 8 and 9 PI to determine if the BBB was permeable to macromolecules. Animals were euthanized on day 10 PI and brain homogenates were studied by Western analysis to detect rabbit Ig leakage into the neuropil. Administration of anti-ICOS antibodies inhibited opening of the BBB. T hese data provide further evidence that costimulation of recently activated PLP-specific T cells through the ICOS/B7RP-1 pathway is a critical step in the immunopathogenesis of EAE.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

Various publications are cited herein, the disclosures of which are incorporated by reference in their entireties.

What is claimed is:

1. A method of treating a multiple sclerosis in a patient, comprising administering to the patient in need of such treatment an ICOS-B7RP-1 inhibitor in an amount effective for treating the demyelinating inflammatory disorder, wherein the ICOS-B7RP-1 inhibitor is an anti-ICOS antibody.

2. The method of claim 1, wherein the ICOS-B7RP-1 inhibitor is administered during a period of relapse, during a period of remission, or during chronic progressive multiple sclerosis in said patient.

3. The method of claim 1, further comprising administering a second therapeutic agent selected from the group consisting of an immunosuppressive agent and a biological response modifier.

4. The method of claim 3, wherein the immunosuppressive agent is cyclosporine, FK506, rapamycin, or prednisone.

5. The method of claim 3, wherein the biological response modifier is an interleukin or an antibody.

6. The method of claim 5, wherein the interleukin is interleukin 4.

7. The method of claim 5, wherein the antibody is immunospecific to CCR1, RANTES, MCP-1, MIP-2, Interleukin-1α, interleukin-1β, interleukin-6, interleukin-12p35, CD28, CTLA-4 or IFN-γ.

8. The method of claim 3, wherein the second therapeutic agent is administered concurrently with the ICOS-B7RP- l inhibitor.

9. A method of identifying a candidate ICOS-B7RP-1 inhibitor, comprising:
(a) contacting an ICOS polypeptide with a B7RP-1 polypeptide expressed on an endothelial cell surface and a test compound, under conditions that, in the absence of the test compound, allow the ICOS polypeptide to bind to the B7RP-1 polypeptide and thereby form an ICOS-B7RP-1 complex; and
(b) determining whether ICOS-B7RP-1 complex formation is inhibited by the test compound;
wherein inhibition of ICOS-B7RP-1 complex formation by the test compound identifies the test compound as a candidate ICOS-B7RP-1 inhibitor.

10. The method of claim 9, wherein the ICOS polypeptide is expressed on a T cell.

11. The method of claim 9, wherein the ICOS polypeptide is immobilized on a solid surface.

12. The method of claim 11, wherein the ICOS polypeptide is present in a cell membrane, which cell membrane is immobilized on the solid surface.

13. The method of claim 9, wherein determining whether ICOS-B7RP-1 complex formation is inhibited by the test compound comprises measuring the amount of binding between ICOS and B7RP-1 or measuring ICOS-B7RP-1 pathway activation.

14. A method of identifying a candidate ICOS-B7RP-1 inhibitor, comprising:
(a) identifying a test compound as a candidate ICOS-B7RP-1 inhibitor by the method of claim 9;
(b) contacting a T-cell, capable of being activated by B7RP-1, with B7RP-1 present on an endothelial cell surface, in the presence of the test compound; and
(c) determining whether a lower level of ICOS-B7RP-1 activity occurs in the T-cell after said contacting relative to a control T-cell contacted with B7RP-1 in the absence of the test compound;
wherein a lower level of activity identifies the test compound as a candidate ICOS-B7RP-1 inhibitor.

15. The method of claim 14, wherein determining whether a lower level of ICOS-B7RP-1 activity occurs in the T-cell comprises measuring ICOS pathway activation.

16. The method of claim 14, wherein determining whether a lower level of ICOS-B7RP-1 activity occurs in the T-cell comprises measuring T cell activation.

17. The method of claim 16, wherein T cell activation is evidenced by the ability of the T cell to traverse an in vitro model of the blood brain barrier.

18. A method of identifying a candidate ICOS-B7RP-1 inhibitor, comprising:
(a) identifying a test compound as a candidate ICOS-B7RP-1 inhibitor by the method of claim 9;
(b) administering to a model animal with experimental allergic encephalomyelitis the test compound during the efferent stage of said experimental allergic encephalomyelitis; and
(c) determining whether the test compound abrogates a central nervous system phenotype of experimental allergic encephalomyelitis,
wherein abrogation of a central nervous system phenotype of experimental allergic encephalomyelitis identifies the test compound as a candidate ICOS-B7RP-1 inhibitor.

19. The method of claim 18, wherein determining whether the test compound abrogates a central nervous system phenotype of experimental allergic encephalomyelitis comprises (i) determining whether ICOS positive T cells traverse the blood brain barrier of said model animal at a reduced rate relative to a model animal with experimental allergic encephalomyelitis to whom the test compound is not administered; (ii) determining whether brain inflammation is reduced in said model animal relative to a model animal with experimental allergic encephalomyelitis to whom the test compound is not administered; or (iii) determining whether physical symptoms of experimental allergic encephalomyelitis are reduced in the model animal relative to a model animal with experimental allergic encephalomyelitis to whom the test compound is not administered.

20. A method of identifying a candidate ICOS-B7RP-1 inhibitor, comprising:
(a) contacting a T-cell, capable of being activated by B7RP-1, with B7RP-1 present on an endothelial cell surface, in the presence of a test compound; and
(b) determining whether a lower level of ICOS-B7RP-1 activity occurs in the T-cell after said contacting relative to a control T-cell contacted with B7RP-1 in the absence of the test compound;
wherein a lower level of activity identifies the test compound as a candidate ICOS-B7RP-1 inhibitor.

21. The method of claim 20, wherein T cell activation is evidenced by the ability of the T cell to traverse an in vitro model of the blood brain barrier.

22. A method of identifying a candidate ICOS-B7RP-1 inhibitor, comprising:
(a) identifying a test compound as a candidate ICOS-B7RP-1 inhibitor by the method of claim 20;
(b) administering to a model animal with experimental allergic encephalomyelitis the test compound during the efferent stage of said experimental allergic encephalomyelitis; and
(c) determining whether the test compound abrogates a central nervous system phenotype of experimental allergic encephalomyelitis,
wherein abrogation of a central nervous system phenotype of experimental allergic encephalomyelitis identifies the test compound as a candidate ICOS-B7RP-1 inhibitor.

23. The method of claim 22, wherein determining whether the test compound abrogates a central nervous system phenotype of experimental allergic encephalomyelitis comprises (i) determining whether ICOS positive T cells traverse the blood brain barrier of said model animal at a reduced rate relative to a model animal with experimental allergic encephalomyelitis to whom the test compound is not administered; (ii) determining whether brain inflammation is reduced in said model animal relative to a model animal with experimental allergic encephalomyelitis to whom the test compound is not administered; or (iii) determining whether physical symptoms of experimental allergic encephalomyelitis are reduced in the model animal relative to a model animal with experimental allergic encephalomyelitis to whom the test compound is not administered.

24. A method of identifying a candidate ICOS-B7RP-1 inhibitor, comprising:
(a) administering to a model animal with experimental allergic encephalomyelitis a test compound during the efferent stage of said experimental allergic encephalomyelitis; and
(b) determining whether the test compound abrogates a central nervous system phenotype of experimental allergic encephalomyelitis,
wherein abrogation of a central nervous system phenotype of experimental allergic encephalomyelitis identifies the test compound as a candidate ICOS-B7RP-1 inhibitor.

25. The method of claim 24, wherein determining whether the test compound abrogates a central nervous system phenotype of experimental allergic encephalomyelitis comprises determining (i) determining whether ICOS positive T cells traverse the blood brain barrier of said model animal at a reduced rate relative to a model animal with experimental allergic encephalomyelitis to whom the test compound is not administered; (ii) determining whether brain inflammation is reduced in said model animal relative to a model animal with experimental allergic encephalomyelitis to whom the test compound is not administered; or (iii) determining whether physical symptoms of experimental allergic encephalomyelitis are reduced in the model animal relative to a model animal with experimental allergic encephalomyelitis to whom the test compound is not administered.

26. The method of claim 24, further comprising, prior to step (a), identifying a suitable test compound by a method comprising:
(a) contacting an ICOS polypeptide with a B7RP-1 polypeptide and a molecule, under conditions that, in the absence of the molecule, allow the ICOS polypeptide to bind to the B7RP-1 polypeptide and thereby form an ICOS-B7RP-1 complex; and
(b) determining whether ICOS-B7RP-1 complex formation is inhibited by the molecule;
wherein inhibition of ICOS-B7RP-1 complex formation by the molecule identifies the molecule as a suitable test compound.
